(19) United States
(12) Patent Application Publication
(10) Pub. No.: US 2019/0076466 A1
(43) Pub. Date: Mar. 14, 2019

(54) REDUCED SODIUM POLOXAMER-188 FORMULATIONS AND METHODS FOR USE

(71) Applicant: MAST THERAPEUTICS, INC., San Diego, CA (US)

(72) Inventors: R. Martin Emanuele, San Diego, CA (US); William Hoye, Carlsbad, CA (US); John Arthur, Encinitas, CA (US); Stewart Smith, San Diego, CA (US); Scott Stern, San Diego, CA (US)

(73) Assignee: LIFERAFT BIOSCIENCES, Mt. Pleasant, SC (US)

(21) Appl. No.: 15/742,858

(22) PCT Filed: Jul. 7, 2016
(86) PCT No.: PCT/US16/41374

§ 371 Date: Jan. 8, 2018

Related U.S. Application Data
(60) Provisional application No. 62/189,580, filed on Jul. 7, 2015.
(60) Provisional application No. 62/238,059, filed on Oct. 6, 2015.

(51) Int. Cl. A61K 31/765; A61K 47/36; A61K 9/00; A61K 9/08

(52) U.S. Cl. CPC A61K 31/765; A61K 9/08; A61K 9/0019; A61K 47/36

(57) ABSTRACT

Provided is a sterile, injectable solution comprising: poloxamer 188 and water for injection, wherein the sterile, injectable solution is reduced in sodium and/or substantially sodium-free. Also provided are methods for using the solution.

### RELATED APPLICATIONS

This application claims the benefit of priority to U.S. Ser. No. 62/189,580, filed Jul. 7, 2015, and U.S. Ser. No. 62/238,059, filed Oct. 6, 2015, which are incorporated herein by reference in their entireties

### FIELD OF THE INVENTION

Disclosed herein are formulations of poloxamer-188, including reduced sodium formulations and substantially sodium-free formulations for use in the field of therapeutics, particularly heart failure.

### BACKGROUND OF THE INVENTION

The average American eats more than 3.4 grams of sodium each day which is above the recommended daily dose of 2.3 g/day (Dietary Guidelines For Americans 2015-2020 8^{th} edition, found at health.gov). Evidence suggests, however, that the current recommended daily dose of sodium in the U.S. is associated with hypertension and other cardiovascular and renal abnormalities (Kaplan, N. M., Am J. Clin Nutr 2000; 71:1020-6). The American Heart Association recommends that people should not eat more than 1.5 g of salt per day in order to prevent high blood pressure and improve heart health (see American Heart Association web site).

Another contributor to salt intake are medicines. George et al. indicate that medicines containing sodium, such as effervescent and soluble medicines were statistically linked to an increased chance of adverse cardiovascular events when compared with people taking standard formulations of the same drugs (George J et al., BMJ 2013; 347:f6954). Thus, medicines may be an unexpected source of sodium in a population of people who need to closely watch their sodium intake.

Poloxamer-188 has rheologic, anti-thrombotic, anti-inflammatory and cytoprotective activities, and has been indicated as a potential treatment for a number of diseases and conditions, including circulatory diseases, pathologic hydrophobic interactions in blood, inflammation, stroke, heart failure, venous occlusive crisis (VOC) associated with sickle cell disease, kidney failure, ischemic/reperfusion injury, physical trauma, electric shock, radiation, osmotic stress, myocardial infarction, burns, frost bite, muscular dystrophy, hemorrhagic shock, hemo-concentration, amyloid oligomer toxicity, spinal cord injury. Poloxamer-188 is currently being investigated in clinical trials for use in sickle cell disease (shortening the duration of vaso-occlusive crisis) and in heart failure (see, U.S. Pat. Nos. 5,605,687; 5,696,298; U.S. Ser. Nos. 12/814,953; 14/793,670; 12/672,907; 14/793,662; 14/793,730; 13/783,158; 15/029,614 and Hunter et al., Ann, Clin. Lab. Sci. 2010; 40(2): 115-125); all incorporated herein by reference.

Poloxamers including poloxamer 188 (and purified poloxamer 188) are polymeric molecules with little to no oral bioavailability and generally require intravenous administration for therapeutic use. Poloxamer 188 (and purified poloxamer 188) have limited potency and require concentrations in the circulation of up to 5.0 mg/ml for optimal activity. Accordingly, formulations of poloxamer 188 suitable for therapeutic use must be sufficiently concentrated to enable achievement of the target concentrations in a physiologically tolerable volume of fluid. PCT Publication no. WO 1994/08596 describes a 15% formulation of sodium citrate buffered poloxamer 188 NF (unpurified poloxamer 188). A similar formulation of 15% sodium citrate buffered purified poloxamer 188 has been used in clinical studies in sickle cell disease patients. (Orringer et al., JAMA (2001); 286(17):2099-2106). These formulations were compatible (isoosmotic) with blood cells, although they have been shown to activate complement (Moghimi et al., Biochemica et Biophysica Acta (2004); 1689: 103-113). Because the formulations are also injected directly into the circulation, the formulation must be suitable for sterilization and have limited capacity to support microbial growth. In addition since poloxamer 188 readily decomposes in the presence of oxygen, the formulation must be stable from oxidation.

Previous formulations of poloxamer 188 NF and purified poloxamer 188 have achieved most of the aforementioned goals using a 15% w/v formulation with sodium citrate (as an anti-oxidant) and sodium chloride (to achieve isotonicity). (WO 1994/08596) However, as mentioned above, recent studies have shown that drugs formulated with sodium salts result in an increased risk of cardiovascular events. (George et al., (2013); BMJ 347: f6954). Furthermore, in certain clinical conditions such as heart failure, liver disease, and kidney failure, use of a 15% solution of poloxamer may require administration of an unacceptable fluid volume to achieve the intended therapeutic concentration. Thus, there is a need for formulations of poloxamer 188 that are reduced in sodium and/or substantially sodium free and which are at a higher concentrations of poloxamer 188 than 15% to enable administration of poloxamer 188 using lower fluid volumes.

### SUMMARY OF THE INVENTION

Disclosed herein are sterile, stable, injectable solutions and/or pharmaceutical compositions containing poloxamer 188 and water for injection, wherein the sterile, stable injectable solution is reduced in sodium or substantially sodium-free; the poloxamer 188 is at a concentration greater than 15% w/v; and the sterile, injectable solution has a pH of from about 4 to about 8. In aspects of this embodiment the poloxamer 188 can be unpurified, or purified. In other aspects the purified poloxamer is long circulating material free (LCMF).

In some aspects the poloxamer 188 has the formula:

HO(CH₂CH₂O)_{a′}—[CH(CH₃)CH₂O]_{b}—(CH₂CH₂O)ₐH;

each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe [CH(CH₃)CH₂O]_{b} is between approximately 1,300 to 2,300 Daltons;
no more than 1.5% of the total components in the distribution of the co-polymer are low molecular weight components having an average molecular weight of less than 4,500 Daltons;

In some embodiments the poloxamer has the formula:

HO(CH₂CH₂O)_{a′}—[CH(CH₃)CH₂O]_{b}—(CH₂CH₂O)ₐH;

each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe [CH(CH₃)CH₂O]_{b} is between approximately 1,300 to 2,300 Daltons;
no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons; the polydispersity value of the copolymer is less than approximately 1.07 or less than 1.07.

In some embodiments, the poloxamer 188 is LCMF with the following formula:

HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe [CH(CH₃)CH₂O]_{b} is between approximately 1,300 to 2,300 Daltons;
no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons;

the polydispersity value of the copolymer is less than approximately 1.07 or less than 1.07; and

the circulating half-life of the co-polymer, when administered to a subject, is no more than 5.0-fold longer than the circulating half-life of the main component in the distribution of the co-polymer.

In some embodiments, the LCMF is produced by a method comprising:

admixing a solution of poloxamer 188 in a first alkanol with an extraction solvent comprising a second alkanol and supercritical carbon dioxide under a temperature and pressure to maintain the supercritical carbon dioxide for a first defined period, wherein:
the temperature is above the critical temperature of carbon dioxide but is no more than 40° C.;
the pressure is 220 bars to 280 bars; and
the alkanol is provided at an alkanol concentration that is 7% to 8% by weight of the total extraction solvent; and

increasing the concentration of the second alkanol in the extraction solvent a plurality of times in gradient steps over time of the extraction method, wherein:

each plurality of times occurs for a further defined period; and

in each successive step, the alkanol concentration is increased 1-2% compared to the previous concentration of the second alkanol; and removing the extraction solvent from the extractor vessel to thereby remove the extracted material from the poloxamer preparation.

In some embodiments, the solutions/pharmaceutical contain poloxamer 188 at concentrations greater than about 15% w/v up to about 30% w/v, greater than about 15 w/v to about 25% w/v, greater than 20% w/v, from about 20% to about 25% w/v, about 20% w/v, about 22.5% w/v, or about 25% w/v.

In some embodiments, the solutions/pharmaceutical compositions contain magnesium salts such as, without limitation, magnesium acetate, magnesium aluminate, magnesium borate, magnesium bicarbonate, magnesium carbonate, magnesium chloride, magnesium citrate, magnesium gluconate, magnesium hydroxide, magnesium lactate, magnesium metasilicate aluminate, magnesium oxide, magnesium phthalate, magnesium phosphate, magnesium silicate, magnesium stearate, magnesium succinate, magnesium tartrate, and mixtures thereof. In some aspects, the solutions/pharmaceutical compositions contain magnesium chloride. In some aspects, the magnesium chloride is at a concentration of 0.61 mg/mL.

In some embodiments, the solutions/pharmaceutical compositions contain one or more tonicity agents such as, without limitation, glucose, glycerin (glycerol), dextrose, sucrose, xylitol, fructose, mannitol, sorbitol, mannose, potassium salts, calcium salts, and magnesium salts. In other aspects, the tonicity agent is a magnesium salt such as, without limitation magnesium acetate, magnesium aluminate, magnesium borate, magnesium bicarbonate, magnesium carbonate, magnesium chloride, magnesium citrate, magnesium gluconate, magnesium hydroxide, magnesium lactate, magnesium metasilicate aluminate, magnesium oxide, magnesium phthalate, magnesium phosphate, magnesium silicate, magnesium stearate, magnesium succinate, magnesium tartrate, and mixtures thereof. In some aspects of this embodiment, the tonicity agents are at a concentration of slightly greater than 0, 1 mM to about 10 mM, 1 to about 5 mM, or 1 mM to about 2 mM.

In some embodiments the solutions/pharmaceutical compositions contain an antioxidant. In some aspects, the antioxidant is chosen from, without limitation, cysteine, citric acid, dextrose, dithiothreitol, histidine, malic acid, mannitol, methionine, metabisulfate, and tartaric acid. In some aspects, the antioxidant is at a concentration from about 0.1 mM to about 10 mM.

In some embodiments, the solutions/pharmaceutical compositions described herein can have a pH from 4-8 or about 6 to 8, or about 7 to about 7.2.

In yet other embodiments, the solutions/pharmaceutical compositions disclosed herein, contain a buffer, In some aspects of this embodiment, the buffer is chosen from, without limitation, citrate buffer (pH about 2); citrate buffer (pH about 5); citrate buffer (pH about 6.3); phosphate buffer (pH about 7.2); phosphate buffer (pH about 9); borate buffer (pH about 9); borate buffer (pH about 10); succinate buffer (pH about 5.6); histidine buffer (pH about 6.1); carbonate buffer (pH about 6.3); acetate buffer (pH about 7.2), meglumine, and combinations thereof. In some aspects, the buffer is citric acid and meglumine at an adjusted pH of about 6, or about 7 to about 7.2.

In some embodiments the solutions/pharmaceutical compositions disclosed herein contain a pH adjusting agent, such as aqueous HCl, ammonium hydroxide, meglumine and mixtures thereof.

In other embodiments, the solutions/pharmaceutical compositions disclosed herein have an osmolality of between about 100 and about 2000 mOSm/kg, or between 300 and about 1500 mOSm/kg, or between about 300 and about 500 mOSm/kg, or between about 270 and about 1500 mOSm/kg, or between about 270 and about 500 mOSm/kg, or greater than 400 mOSm/kg.

In some embodiments, the solutions/pharmaceutical compositions disclosed herein contain one or more other active ingredients. In some aspects, the other active ingredients are chosen from, without limitation, acetaminophen, adenosine, hydroxurea, amiodarone HCl, atropine sulfate, bumetanide, cefazolin, chlorothiazide sodium, dexamethasone sodium phosphate, digoxin HCl, dobutamine diphenhydramine HCl, dopamine HCl, enalapril maleate, epinephrine HCl, fentanyl citrate, furosemide, gentamicin sulfate, heparin sodium, hydrocortisone sodium succinate, isoproterenol HCl, labetalol HCl, lidocaine HCl, mannitol, meperidine HCl, metoprolol tartrate, milrinone, nafcillin sodium, naloxone, nesiritide, norepinephrine bitartrate, ondansetron HCl, phenylephrine HCl, promethazine HCl, quinidine gluconate, and verapamil.

In some embodiments, the solutions/pharmaceutical compositions disclosed herein are packaged in a sealed, pharmaceutically acceptable container such as a vial that can be, without limitation, flint glass or borosilicate glass. In some aspects the acceptable container is an infusion bag, such as without limitation, an infusion bag with PVC, PVC with DEHP, PVC with TOTM, polyolefin, polypropylene or EVA. In some aspects, the pharmaceutically acceptable container is sealed in a foil pouch wherein the atmosphere within the sealed foil pouch containing the pharmaceutically acceptable container comprises argon, nitrogen, and/or carbon dioxide or an inert atmosphere.

In some aspects the sealed solutions/pharmaceutical compositions contain no more than about 2.0 mg/mL dissolved oxygen.

Also disclosed herein are methods of treating a disease or condition in a subject, by administering a therapeutically effective amount of the solutions/pharmaceutical compositions disclosed herein, including, without limitation, diseases and conditions such as acute coronary syndromes, limb ischemia, shock, stroke, heart failure, coronary artery disease, muscular dystrophy, circulatory diseases, pathologic hydrophobic interactions in blood, inflammation, sickle cell disease, venous occlusive crisis, acute chest syndrome, inflammation, pain, neurodegenerative diseases, macular degeneration, thrombosis, kidney failure, burns, spinal cord injuries, ischemic/reperfusion injury, myocardial infarction, hemo-concentration, amyloid oligomer toxicity, diabetic retinopathy, diabetic peripheral vascular disease, sudden hearing loss, peripheral vascular disease, cerebral ischemia, transient ischemic attacks, critical limb ischemia, respiratory distress syndrome (RDS), adult respiratory distress syndrome (ARDS) circulatory diseases, or pathologic hydrophobic interactions in blood. In some aspects of this method, the solutions/pharmaceutical compositions are administered with one or more active ingredients such as acetaminophen, adenosine, amiodarone HCl, atropine sulfate, bumetanide, cefazolin, chlorothiazide sodium, dexamethasone sodium phosphate, digoxin HCl, dobutamine diphenhydramine HCl, dopamine HCl, enalapril maleate, epinephrine HCl, fentanyl citrate, furosemide, gentamicin sulfate, heparin sodium, hydrocortisone sodium succinate, isoproterenol HCl, labetalol HCl, lidocaine HCl, mannitol, meperidine HCl, metoprolol tartrate, milrinone, nafcillin sodium, naloxone, nesiritide, norepinephrine bitartrate, ondansetron HCl, phenylephrine HCl, promethazine HCl, quinidine gluconate, hydroxyurea, and verapamil.

In some embodiments, one or more of the tonicity agent, antioxidant, buffer, or pH adjusting agent are substantially free of sodium.

In some embodiments, the solutions/pharmaceutical compositions are reduced in sodium.

In some embodiments, the solutions/pharmaceutical compositions are substantially sodium free. In an aspect of this embodiment, the solutions/pharmaceutical compositions have less than 0.1 mg/mL sodium.

In some embodiments, the solutions/pharmaceutical compositions are stable at 5° C.±3° C. for at least 6 months, or at least 12 months, or at least 24 months.

In some embodiments the solutions/pharmaceutical compositions do not significantly activate complement when administered to a subject as evidenced by clinical signs and symptoms, such as hypotension, tachycardia and shortness of breath.

### BRIEF DESCRIPTION OF THE DRAWINGS

FIG. 1 shows that plasma TnI levels were progressively reduced after the administration of either low or high dose of substantially sodium free poloxamer-188 LCMF.

FIG. 2 shows plasma nt-pro BNP levels were progressively reduced after the administration of either low or high dose of substantially sodium-free poloxamer-188 LCMF.

FIG. 3 shows complement activation (Bb and sC5b-9) in human normal serums (average ±SD) by various Poloxamer-188 LCMF formulations as described in Table 10 and PBS (P), and Zymosan (0.1 mg/ml: Z).

### DETAILED DESCRIPTION OF THE INVENTION

The present disclosure describes stable pharmaceutical compositions of poloxamer-188 that are reduced in sodium, or are in some embodiments substantially sodium free compared to previous formulations of poloxamer-188, such as described in WO 1994/98596 which discloses a 15% formulation of poloxamer-188 in water with 150 mg/ml poloxamer-188, 3.08 mg/ml sodium chloride, 2.38 mg/ml sodium citrate (dihydrate) and 0.366 citric acid anhydrous for a sodium concentration of about 1.77 mg/ml; WO 2016/007542 discloses a long circulating material free (LCMF) poloxamer-188 formulation for treating heart failure that contains 150 mg/ml LCMF poloxamer-188; 3.08 mg/ml sodium chloride; 2.38 mg/ml sodium citrate (dihydrate), 0.366 mg/ml citric acid and water for injection. The pH of the solution is about 6.0, has an osmolality of about 312 mOsm/L and contains about 1.77 mg/ml sodium; and U.S. Publication No. 2011/0044929 discloses a poloxamer-188 formulation that contains 5% w/v poloxamer-188, 5 mM Tris-HCl pH 8.0, and 0.9% w/v sodium chloride injection, and, thus, contains about 3.5 mg/ml sodium.

The formulations of poloxamer-188 described herein are also at higher concentrations than previous formulations of poloxamer 188 known in the art (15% poloxamer 188) to enable administration of the polymer to subjects at reduced fluid volumes.

These formulations combine poloxamer-188 (purified or unpurified) with excipients. These excipients may serve a variety of functions, such as without limitation, solubilizing, tonicity adjustment, suspending, diluting, buffering, and stabilizing the poloxamer-188. The formulations disclosed herein result in a drug product that is stable, efficacious, easy to administer, compatible with blood, and well tolerated.

The formulations disclosed herein also prevent the poloxamer 188 from being degraded or destroyed by atmospheric oxygen. When added to blood at therapeutic concentrations, the formulations have a low potential for complement activation. The formulations disclosed herein can be sterilized and are free of particulate matter. The formulations have a low potential to support microbial growth and are stable from microbiologic contamination during at least six months of storage (data not shown).

Because of their low sodium content and in some cases high concentration of poloxamer-188, the formulations are useful in conditions, such as heart failure, and/or kidney failure or other conditions where excess volume and/or high sodium intake may be detrimental to a patient's health.

For treatment of diseases and conditions in which the patient is sensitive to increased fluid volume, the concentration of poloxamer-188 in the formulation needed to be maximized. Thus, the aqueous solubility of purified poloxamer-188 (LCMF) was determined by adding the API to water in 50 mg increments from 150 mg/ml to 400 mg/ml with an additional sample prepped at 500 mg/ml. It was determined that purified poloxamer-188 is soluble in water up to 400 mg/ml. The 500 mg/ml sample did not form a solution but instead formed a clear gel (data not shown) and thus was not suitable for injection.

During the solubility study, it was observed that as the solution concentrations increased so did their viscosities. Viscosity and injectability (force required for injection) were determined over a range of concentrations (Table 17). Both properties increased exponentially when the concentration was increased linearly.

The effect of increased viscosity on the rate of delivery of poloxamer-188 was determined and the data indicated the formulations tested met the needs of injectability (Tables 18 and 19). There was no observable correlation between concentration of poloxamer-188 LCMF and rate of delivery. Density values over a range of poloxamer-188 LCMF concentrations showed a linear increase with increased poloxamer-188 LCMF concentrations (data not shown).

The tonicity of a parenteral solution is of critical importance. If a hypotonic solution is given intravenously the red blood cells will take in water in an attempt to equalize the osmotic pressure, causing them to swell and potentially burst. The opposite is true for a hypertonic solution with water exiting the blood cells causing them to shrink and shrivel (crenate). The osmolality of poloxamer-188 LCMF in water was evaluated over a range of 25 mg/ml to 400 mg/ml. As the concentration of poloxamer-188 in the solutions increased an exponential increase in osmolality was observed (Table 7). Isotonic solutions are typically defined as possessing an osmolality value between 270-300 mOsm/kg water. Any solutions with osmolality values below 270 mOsm/kg or above 300 mOsm/kg are considered hypotonic and hypertonic respectively. These results surprisingly suggested a concentration between 200 and 250 mg/mL should be targeted to achieve an isotonic solution with minimal to no tonicity adjusting agents added. Six different concentrations of poloxamer-188 LCMF (15%-40%) were tested on whole blood to determine if any crenation of red blood cells occurred (Tables 8 and 9). It was found that all hypertonic solutions made with NaCl solution but no poloxamer-188 crenated cells. Solutions containing poloxamer-188 LCMF showed no signs of crenated cells indicating that poloxamer-188 protects red blood cells even in non-isotonic solutions, and, thus, poloxamer-188 besides being the API also may be acting as a tonicity agent in the hypertonic formulations disclosed herein.

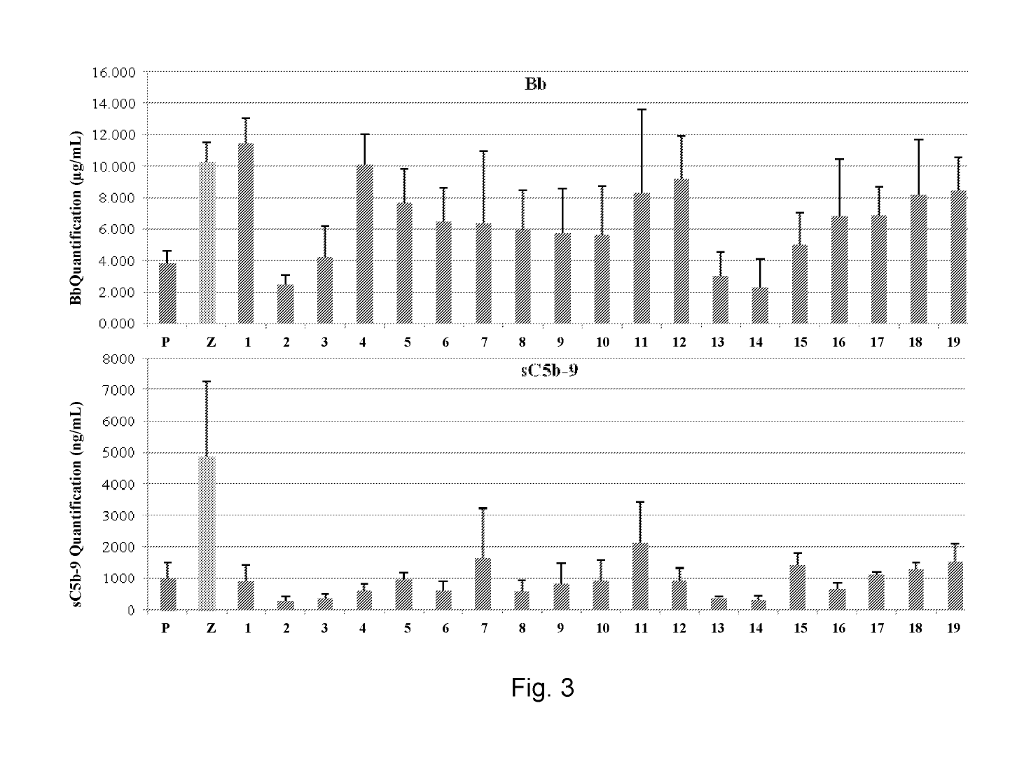

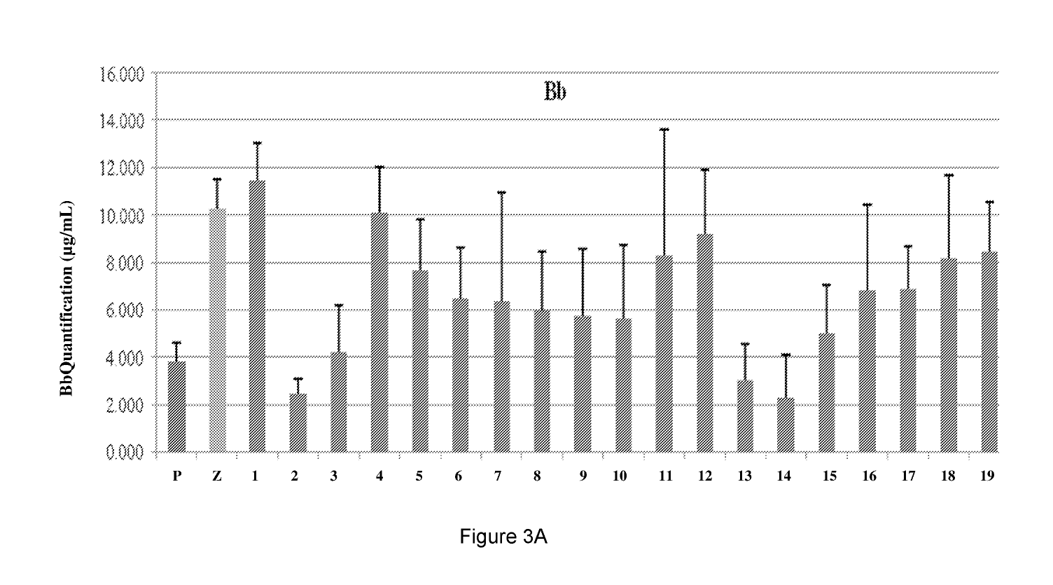

Various substantially sodium-free formulations containing poloxamer-188 LCMF were tested for the ex vivo activation and quantification of human serum complement factors,—the Bb fragment and sC5b-9 complex—using Enzyme-linked Immunosorbent Assays (ELISA)—see Table 10 and FIG. 3. Serum samples from 3 healthy human donors were collected and activated with 19 test formulations or control items and analyzed for Bb and sC5b-9 content. A total of 19 formulations were used to activate 3 individual normal human serum samples which were analyzed by ELISA for Bb and sC5b-9 content. Treatment of serum with the test formulations induced Bb and sC5b-9 levels with varying amplitude. The greatest increase of Bb levels was induced by formulations #1, 4, 5, 12, 17, 18, and 19 with no overlap of standard deviation (SD) intervals compared to PBS-induced levels. The greatest increase of sC5b-9 levels was induced by formulation #11 (2.19-fold increase) with levels in all three normal human serums increasing above their respective PBS-induced levels. However, when comparing averages of all three serum values, no formulation induced sC5b-9 levels above the average background levels, with no overlap of the SD intervals (FIG. 3)

The stability of poloxamer-188 LCMF in buffers was evaluated in citrate, phosphate, acetate, histidine, succinate, tartrate, glycine, sulfate, TRIS, carbonate, and borate buffers over a pH range of 2 to 10 under accelerated conditions to assess the role of pH and buffer species on solution stability of poloxamer-188 LCMF (Table 11). The solutions at high pH displayed greater stability compared to the more acidic solutions. Poloxamer-188 LCMF displayed acceptable stability in pH range 6-8. Buffer species showed a potential to affect stability. The citrate buffered formulations appeared to have the least amount of fluctuation in pH and osmolality when compared to other buffer species at similar poloxamer-188 LCMF concentrations. With regards to initial concentration, it is shown herein that an increase in poloxamer-188 LCMF concentration does not increase the rate of degradation (Table 11).

Particle studies were performed to determine the cause of particle formation that was seen in a batch of a substantially sodium-free 15% poloxamer-188 LCMF. Testing of nine different poloxamer-188 LCMF formulations indicated that the precipitation was probably due to a complexation between magnesium, citrate, and poloxamer (Table 6).

The antioxidants ascorbic acid, cysteine, citric acid, dextrose, dithiothreitol, histidine, malic acid, mannitol, methionine, sodium metabisulfate, and tartaric acid were evaluated in 2% and 25% solutions of poloxamer-188 LCMF in 20 mM phosphate buffer at pH 6 were evaluated at t=0 and t=1 month at 40° C. The results are shown in Table C. The data indicates that good stability at 1 month was achieved with mannitol, methionine and citrate for concentrated solutions of poloxamer of 25%.

Disclosed herein are pharmaceutical formulations of poloxamer-188 for injection. These formulations are reduced in sodium and/or substantially sodium free as compared to prior art poloxamer-188 formulations. The formulations contain concentrations of poloxamer-188 above 15% to enable delivery of smaller volumes of fluid as well as low amounts of sodium to prevent adverse effects in patient populations that would be susceptible to increased fluid volumes and/or sodium content, such as, without limitation, heart patients, kidney failure patients, and sickle cell disease patients.

Disclosed herein are sterile, injectable solutions comprising: poloxamer-188 and water for injection, wherein the sterile, injectable solutions are reduced in sodium as compared to prior art formulations and/or substantially sodium-free; the poloxamer-188 is stable from oxidative decomposition; and the sterile, injectable solution has a pH of from about 4 to about 8, or about 6 to about 8.

The disclosed formulations can be given in combination with other agents, for example, without limitation, hydroxyurea, pain medications, such as opioids, antithrombotics, such as, without limitation t-PA, diuretics, loop diuretic, potassium sparing agents, a vasodilator, such as without limitation nitrates, nitrites, and Sildenafil, ACE inhibitors, angiotensin receptor blockers, angiotensin II antagonists, aldosterone antagonist, a positive inotrophic agent, a phosphodiesterase inhibitor, a beta-adrenergic receptor antagonist, a calcium channel blocker, an alpha blocker, a central alpha antagonist, a statin, a cardiac glycoside, digoxin, chlorthalidone, amlodipine, lisinopril, doxazosin, anti-inflammatories, selectin inhibitors, such as without limitation, Rivipansel, SelGI, Sevuparin, Propranolol, Regadenoson, NKTT120, Montelukast, Zileuton, IVIg, Simvastatin, arginine butyrate, HQK-1001, Pomalidomide, SCD-101, MP4CO, Sanguinate, Senicapoc, Aes-103, heparins and anticoagulants, such as without limitation, Dalteparin, unfractionated heparin, Rivaroxiban, Apixiban, n-acetyl cysteine, antiplatelet agents, such as, without limitation, Prasugrel, aspirin, and Ticagrelor and a combination of these agents. In some embodiments, the poloxamer 188 formulation can be given prior to, at the same time, or subsequent to the other agents, or combinations of all three.

Also provided are methods of treating a disease or condition in a subject, comprising administering the solution described herein. In some embodiments, the disease or condition is selected from acute coronary syndromes, limb ischemia, shock, stroke, heart failure, including without limitation, systolic, diastolic, congestive, and cardiomyopathies, coronary artery disease, muscular dystrophy, circulatory diseases, pathologic hydrophobic interactions in blood, inflammation, sickle cell disease, such as venous occlusive crisis, and acute chest syndrome, inflammation, pain, neurodegenerative diseases, macular degeneration, thrombosis, kidney failure, burns, spinal cord injuries, ischemic/reperfusion injury, myocardial infarction, hemo-concentration, amyloid oligomer toxicity, diabetic retinopathy, diabetic peripheral vascular disease, sudden hearing loss, peripheral vascular disease, cerebral ischemia, transient ischemic attacks, critical limb ischemia, respiratory distress syndrome (RDS), and adult respiratory distress syndrome (ARDS).

Unless defined otherwise, all technical and scientific terms used herein have the same meaning as is commonly understood by one of skill in the art to which the invention(s) belong. All patents, patent applications, published applications and publications, databases, websites and other published materials referred to throughout the entire disclosure herein, unless noted otherwise, are incorporated herein by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “Poloxamer 188” (also called P-188 or P188) refers to a polyoxyethylene/polyoxypropylene copolymer that has the following chemical formula:

HO(CH₂CH₂O)_{a′}—[CH(CH₃)CH₂O]_{b}—(CH₂CH₂O)ₐH, where:

a′ and a can be the same or different and each is an integer such that the hydrophile portion represented by (C₂H₄O) (i.e., the polyoxyethylene portion of the copolymer) constitutes approximately 60% to 90%, such as approximately 80% or 81%; and b is an integer such that the hydrophobe represented by (C₃H₆O) has a molecular weight of approximately 1,300 to 2,300 Da, such as 1,400 to 2,000 Da, for example approximately 1,750 Da. For example, a is about 79 and b is approximately or is 28. The average total molecular weight of the compound is approximately, 7200-9700 Da, or approximately 7,680 to 9,510 Da, or 7350 to 8850 Da such as generally 8,400-8,800 Da, for example about or at 8,400 Da. or about 8500 Da. The polyoxyethylene-polyoxypropylene-polyoxyethylene weight ratio of is approximately 4:2:4. According to specifications, P188 has a weight percent of polyoxyethylene of 81.8±1.9%, and an unsaturation level of about 0.010 to 0.034 mEq/g, or for example 0.026±0.008 mEq/g. Unsaturation levels can be measured according to known techniques such as those described by Moghimi et al, Biochimica et Biophysica Acta (2004); 1689: 103-113.

The nomenclature of the polyoxyethylene/polyoxypropylene copolymer relates to its monomeric composition. The first two digits of a poloxamer number, multiplied by 100, gives the approximate molecular weight of the hydrophobic polyoxypropylene block. The last digit, multiplied by 10, gives the approximate weight percent of the hydrophilic polyoxyethylene content. For example, poloxamer 188 describes a polymer containing a polyoxypropylene hydrophobe of about 1,800 Da with a hydrophilic polyoxyethylene block content of about 80% of the total molecular weight.

Poloxamer 188 contains a heterogeneous distribution of polymer species that primarily vary in overall chain length of the polymer, but also include truncated polymer chains with unsaturation, and certain low molecular weight glycols. Included among poloxamer 188 molecules are those that exhibit a species profile (e.g. determined by GPC) containing a main peak and “shoulder” peaks on both sides representing low molecular weight (LMW) polymer species and high molecular weight (HMW) polymer species.

Poloxamers are synthesized in two steps, first by building the polyoxypropylene core, and then by addition of polyoxyethylene to the terminal ends of the polyoxypropylene core. Because of variation in the rates of polymerization during both steps, a poloxamer can contain heterogeneous polymer species of varying molecular weights. The distribution of polymer species can be characterized using standard techniques including, but not limited to, gel permeation chromatography (GPC).

As used herein, “purified poloxamer 188” or “P188-P” or “purified longer circulating material (LCM)-containing poloxamer 188” refers to a poloxamer 188 that has polydispersity value of the poloxamer of less than or about 1.07, such as less than or 1.05 or less than or 1.03, whereby the purified poloxamer 188 has a reduced amount low molecular weight components. A poloxamer 188 in which “low molecular weight material has been removed” or “low molecular weight material has been reduced,” or similar variations thereof, refers to a purified poloxamer 188 in which there is a distribution of low molecular weight components of no more than or less than 3.0%, and generally no more than or less than 2.0% or no more than or less than 1.5% or no more than about 1.0% of the total distribution of components. Typically, such a poloxamer 188 exhibits reduced toxicity compared to forms of poloxamer 188 that contain a higher or greater percentage of low molecular weight components. The poloxamer 188 is purified to remove or reduce low molecular weight components.

An exemplary purified LCM-containing poloxamer 188 is poloxamer 188 available under the trademark FLOCOR® (see, also U.S. Pat. No. 5,696,298, which describes LCM-containing poloxamer 188 and Grindel et al. (2002) Biopharmaceutics & Drug Disposition, 23:87-103). When the purified LCM-containing poloxamer 188 is administered as an intravenous injection to a mammal, particularly a human, GPC analysis of blood obtained from the treated subject exhibits two circulating peaks: a peak designated the main peak that comprises the main component of the polymeric distribution and a peak of higher molecular weight, compared to the main peak, that exhibits a substantially slower rate of clearance (more than 5-fold slower than the main peak, typically more than 30 hours and as much as 70 hours, as shown herein) from the circulation, i.e., a long circulating material (LCM) (Grindel et al. (2002) Biopharmaceutics & Drug Disposition, 23:87-103).

As used herein, “main component” or “main peak” with reference to a poloxamer 188 preparation refers to the species of copolymer molecules that have a molecular weight of less than about 13,000 Da and greater than about 4,500 Da, with an average molecular weight of between about 7200 to 9700 Da, or about 7,680 to 9,510 Da, or 7350 to 8850 Da, such as generally 8,400-8,800 Da, or about 8,200-8,800 Da, for example, about or at 8,400 Da or about 8500 Da. Main peak species include those that elute by gel permeation chromatography (GPC) at between 14 and 15 minutes depending on the chromatography conditions (see U.S. Pat. No. 5,696,298 and Grindel et al., Biopharm Drug Dispos 2002; 23(3):87-103).

As used herein, “low molecular weight” or “LMW” with reference to the species or components of a poloxamer 188 preparation refers to components that have a molecular weight generally less than 4,500 Da. LMW species include those that elute by gel permeation chromatography (GPC) after 15 minutes depending on the chromatography conditions. (see U.S. Pat. No. 5,696,298 and Grindel et al. (2002) Biopharmaceutics & Drug Disposition, 23:87-103)). Such impurities can include low molecular weight poloxamers, poloxamer degradation products (including alcohols, aldehydes, ketones, and hydroperoxides), diblock copolymers, unsaturated polymers, and oligomeric glycols including oligo(ethylene glycol) and oligo(propylene glycol).

As used herein, “high molecular weight” or “HMW” with reference to the species or components of a poloxamer 188 preparation refers to components that have a molecular weight generally greater than 13,000 Da, such as greater than 14,000 Da, greater than 15,000 Da, greater than 16,000 Da or greater. HMW species include those that elute by gel permeation chromatography (GPC) at between 13 and 14 minutes depending on the chromatography conditions (see U.S. Pat. No. 5,696,298 and Grindel et al., Biopharm Drug Dispos 2002; 23(3):87-103).

As used herein, “polydispersity” or “D” refers to the breadth of the molecular weight distribution of a polymer composition. A monodisperse sample is defined as one in which all molecules are identical. In such a case, the polydispersity (Mw/Mn) is 1. Narrow molecular weight standards have a value of D near 1 and a typical polymer has a range of 2 to 5. Some polymers have a polydispersity in excess of 20. Hence, a high polydispersity value indicates a wide variation in size for the population of molecules in a given preparation, while a lower polydispersity value indicates less variation. Methods for assessing polydispersity are known in the art, and include methods as described in U.S. Pat. No. 5,696,298. For example, polydispersity can be determined from chromatograms. It is understood that polydispersity values can vary depending on the particular chromatographic conditions, the molecular weight standards and the size exclusion characteristics of gel permeation columns employed. For purposes herein, reference to polydispersity is as employed in U.S. Pat. No. 5,696,298, as determined from chromatograms obtained using a Model 600E Powerline chromatographic system equipped with a column heater module, a Model 410 refractive index detector, Maxima 820 software package (all from Waters, Div. of Millipore, Milford, Mass.), two LiChrogel PS-40 columns and a LiChrogel PS-20 column in series (EM Science, Gibbstown, N.J.), and polyethylene glycol molecular weight standards (Polymer Laboratories, Inc., Amherst, Mass.). It is within the level of a skilled artisan to convert any polydispersity value that is obtained using a different separation method to the values described herein simply by running a single sample on both systems and then comparing the polydispersity values from each chromatogram.

As used herein, “long circulating material free” or “LCMF” with reference to poloxamer 188 refers to a purified poloxamer 188 preparation that has a reduced amount of low molecular weight components, as described above for purified poloxamer 188, and that, following intravenous administration to a subject, the components of the polymeric distribution clear from the circulation in a more homogeneous manner such that any longer circulating material exhibits a half-life that is no more than 5-fold longer than the circulating half-life of the main peak. Thus, an LCMF is a poloxamer 188 that does not contain components, such as a high molecular weight components or low molecular weight components as described herein, that are or gives rise to a circulating material with a t_{1/2} that is more than 5.0-fold greater than the t_{1/2} of the main component, and generally no more than 4.0, 3.0, 2.0 or 1.5 fold greater than the half-life of the main component in the distribution of the copolymer. In some embodiments the LCMF poloxamer 188 has an unsaturation level of about 0.018 to about 0.034 mEq/g. Typically, an LCMF poloxamer is a poloxamer in which all of the components of the polymeric distribution clear from the circulation at a more homogeneous rate.

As used herein, “distribution of copolymer” refers to the molecular weight distributions of the polymeric molecules in a poloxamer preparation. The distribution of molecular masses can be determined by various techniques known to a skilled artisan, including but not limited to, colligative property measurements, light scattering techniques, viscometry and size exclusion chromatography. In particular, gel permeation chromatography (GPC) methods can be employed that determine molecular weight distribution based on the polymer's hydrodynamic volume. The distribution of molecular weight or mass of a polymer can be summarized by polydispersity. For example, the greater the disparity of molecular weight distributions in a poloxamer, the higher the polydispersity.

As used herein, “impurities” refer to unwanted components in a poloxamer preparation. Typically impurities include LMW components less than 4,500 daltons and high molecular weight components greater than 13,000 daltons.

As used herein “retention time” or t_{R} or RT means the time elapsed between the injection of a sample, such as an LCMF poloxamer 188 sample, onto a reversed phase column for RP-HPLC and the peak response by the evaporative light scattering detector. The retention time is longer when the when the sample is more hydrophobic.

LCM-containing purified poloxamer 188, such as the poloxamer available under the trademark FLOCOR® has a mean retention time (t_{R}) of 9.883 and a k′ of 3.697; whereas the LCMF poloxamer 188 has a mean retention time (t_{R}) of 8.897 and a mean k′ of 3.202.

As used herein, “substantially sodium-free” means that the solution contains less than about 3 parts per million (ppm) sodium or less than about 2 ppm sodium, or less than about 1 ppm sodium, or less than about 0.700 ppm sodium. In some embodiments substantially sodium-free means the solution before administration contains less than about 0.7 μg/ml sodium or less than about 0.5 μg/ml sodium or about less than about 0.3 μg/ml sodium, or about 0.1 μg/ml sodium, or about 0.08 μg/ml, or less than about 0.07 μg/ml sodium or less than about 0.06 μg/ml sodium.

As used herein “reduced sodium” means that the solution contains less than about 1.5 mg/ml, or less than about 1.4 mg/ml, or less than about 1.3 mg/ml, 1.2 mg/ml, or less than about 1.1 mg/ml, or less than about 1.0 mg/ml, or less than about 0.9 mg/ml, or less than about 0.8 mg/ml, or less than about 0.7 mg/ml, or less than about 0.6 mg/ml, or less than about 0.5 mg/ml or less than about 0.4 mg/ml or less than about 0.3 mg/ml, or less than about 0.2 mg/ml, or less than about 0.1 mg/ml, or less than about 0.09 mg/ml or less than about 0.08 mg/ml, or less than about 0.07 mg/ml, or less than about 0.06 mg/ml, or less than about 0.05 mg/ml, or less than about 0.04 mg/ml, or less than about 0.03 mg/ml, or less than about 0.02 mg/ml, or about less than 0.01 mg/ml, or less than about 0.009 mg/ml, or less than about 0.001 mg/ml, or less than about 0.0008 mg/ml sodium, or about 0.0008 mg/ml to about 1.5 mg/ml sodium.

In some embodiments, none of the excipients in the formulation are a sodium salt. In some embodiments, a sodium salt is not used in the preparation of the solution.

Oxidative decomposition is the primary degradation pathway affecting stability of poloxamers. This process generates structural changes to the polymer chain and generates peroxides and carbonyls. “Stable from oxidative decomposition” means that the pH of the solution of poloxamer 188 or or LCMF poloxamer 188 or purified poloxamer 188 is maintained within the range of 5-7 and the acetaldehyde content is below 299 ppm for a period of at least 6 months when stored at room temperature under ambient light. Measurement of acetaldehyde is measured by headspace gas chromatography, such as described by Moghimi et al, Biochimica et Biophysica Acta (2004); 1689:103-113.

As used herein, “complement activation” may be measured by methods known in the art, e.g., (1) peptide binding to C3 and C3 fragments; (2) various hemolytic assays; (3) measurement of C3 convertase-mediated cleavage of C3; (4) measurement of Factor B cleavage by Factor D; and (5) measurement of the two complement split products, SC5b-9 and Bb, using enzyme-linked immunosorbent assay kits. As used herein, a solution induces complement activation following contact with blood if there is a doubling in the plasma level of a complement activation factor such as Clq, ClINH, C3, C4 or Factor B (from the basal or pre-exposure level).

As used herein, “clinically significant complement activation” means a systemic activation of complement as evidenced by clinical signs and symptoms such as, without limitation, hypotension, tachycardia, and shortness of breath.

As used herein, “tonicity agent” or “tonicity adjusting agent” refers to any agent that alters the osmolality of an aqueous solution. Typically, tonicity agents are used to adjust the osmolality of a solution to bring it closer to the osmotic pressure of body fluids, such as blood or plasma.

As used herein, “treatment” refers to ameliorating or reducing symptoms associated with a disease or condition. Treatment means any manner in which the symptoms of a condition, disorder or disease are ameliorated or otherwise beneficially altered. Hence treatment encompasses prophylaxis, therapy and/or cure. Treatment also encompasses any pharmaceutical use of the compositions herein.

As used herein, “treating” a subject having a disease or condition means that a composition or other product provided or described herein is administered to the subject to thereby effect treatment thereof.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, “prevention” or “prophylaxis” refers to methods in which the risk of developing disease or condition is reduced. Prophylaxis includes reduction in the risk of developing a disease or condition and/or a prevention of worsening of symptoms or progression of a disease, or reduction in the risk of worsening of symptoms or progression of a disease.

As used herein an “effective amount” of a compound or composition for treating a particular disease is an amount that is sufficient to ameliorate, or in some manner reduce symptoms to achieve the desired physiological effect. Such amount can be administered as a single dosage or can be administered according to a regimen, whereby it is effective. The effective amount is readily determined by one of skill in the art following routine procedures.

As used herein, “therapeutically effective amount” or “therapeutically effective dose” refers to an agent, compound, material, or composition containing a compound that is at least sufficient to produce a therapeutic effect.

As used herein the term “short term infusion(s)” means an intravenous infusion administered over a period of less than 24 hours.

As used herein, a “single infusion” refers to an infusion that provides an effective amount of a compound or pharmaceutical composition in only one infusion or administration.

As used herein, “disease” or “disorder” or “condition” refers to a pathological condition in an organism resulting from cause or condition including, but not limited to, infections, acquired conditions, genetic conditions, and characterized by identifiable symptoms.

As used herein, “patient” or “subject” to be treated includes humans and or non-human animals, including mammals. Mammals include primates, such as humans, chimpanzees, gorillas and monkeys; domesticated animals, such as dogs, horses, cats, pigs, goats, cows; and rodents such as mice, rats, hamsters and gerbils.

As used herein, the singular forms “a,” “an” and “the” include plural referents unless the context clearly dictates otherwise.

“About” or “approximately” as used herein when referring to a measurable value such as an amount, a temporal duration, and the like, is meant to encompass variations of ±30%-±10%, more preferably ±5%, even more preferably ±1%, and still more preferably ±0.1% from the specified value, as such variations are appropriate to perform the disclosed methods. About also includes the exact amount. Hence “about 0.05 mg/mL” means “about 0.05 mg/mL” and also “0.05 mg/mL.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

Provided herein are sterile, stable, injectable solutions/pharmaceutical compositions, where the solutions/pharmaceutical compositions are reduced in sodium and/or substantially sodium-free and contain poloxamer 188 that can be purified, such as LCMF or unpurified with a pH from about 4 to about 8.

In some embodiments, the solution does not induce significant complement activation following contact with blood.

In some embodiments, the solutions is stable for at least 6 months, 12 months, or 24 months at 5° C.±3° C. (Example 20)

In some embodiments, the poloxamer 188 comprises poloxamer 188, N.F., e.g., a commercially available poloxamer 188. In some embodiments, the poloxamer 188 has a molecular weight of approximately 8400 Daltons, or approximately 8500 Daltons. In some embodiments, the poloxamer 188 is available under the trademarks Pluronic® F-68, Kolliphor® P 188, 80% POE.

In some embodiments, the poloxamer 188 comprises purified poloxamer 188. In some embodiments, the purified poloxamer 188, when administered to a human subject, has a half-life (t_{1/2}) in plasma of about 7 hours. (Grindel et al. (2002) Journal of Pharmaceutical Sciences, 90:1936-1947 (Grindel et al. 2002a) or Grindel et al. (2002) Biopharmaceutics & Drug Disposition, 23:87-103 (Grindel et al. 2002b)). In some embodiments, the purified poloxamer 188 comprises a longer circulating material consisting of higher molecular weight components that had an average molecular weight of about 16,000 Daltons, which exhibited about a 10-fold or more increase in half-life with a t_{1/2} of approximately 70 hours.

In comparison to purified poloxamer 188, non-purified forms of P188 contains a bell-shaped distribution of polymer species, which vary primarily in overall chain length. In addition, various low molecular weight (LMW) components (e.g. glycols and truncated polymers) formed by incomplete polymerization, and high molecular weight (HMW) components (e.g. dimerized polymers) can be present. Typically, characterization of P188 by gel permeation chromatography (GPC) identifies a main peak of P188 with “shoulder” peaks representing the unintended LMW and HMW components (Emanuele and Balasubramaniam (2014) Drugs R D, 14:73-83). For example, the preparation of P188 that is available from BASF (Parsippany, N.J.) has a published structure that is characterized by a hydrophobic block with a molecular weight of approximately 1,750 daltons (Da), POE blocks making up 80% of the polymer by weight, and a total molecular weight of approximately 8,400 Da. The actual compound is composed of the intended POE-POP-POE copolymer, but also contains other molecules which range from a molecular weight of less than 1,000 Da to over 30,000 Da. The molecular diversity and distribution of molecules of commercial poloxamer 188 is illustrated by broad primary and secondary peaks detected using gel permeation chromatography.

In some embodiments, the poloxamer 188 comprises a long circulating material free (LCMF) poloxamer 188, wherein:
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe [CH(CH₃)CH₂O]_{b} is between approximately 1,300 to 2,300 Daltons;
no more than 1.5% of the total components in the distribution of the co-polymer are low molecular weight components having an average molecular weight of less than 4,500 Daltons;
no more than 1.5% of the total components in the distribution of the co-polymer are high molecular weight components having an average molecular weight of greater than 13,000 Daltons;
the polydispersity value of the copolymer is less than approximately 1.07 or less than 1.07; and
the circulating half-life of the co-polymer, when administered to a subject, is no more than 5.0-fold longer than the circulating half-life of the main component in the distribution of the co-polymer.

In some embodiments, all components of the LCMF poloxamer which comprise the polymeric distribution of the co-polymer, have a circulating half-life in the plasma of the subject that is no more than 5 fold or 4.0-fold, or 3.0-fold longer than the circulating half-life of the main component of the co-polymer following intravenous administration to a subject.

In some embodiments, all components in the distribution of the LCMF poloxamer, when administered to a subject, have a circulating half-life in the plasma of the subject that is no more than 4-fold longer than the circulating half-life of the main component in the distribution of the LCMF poloxamer.

In some embodiments, all components in the distribution of the LCMF poloxamer, when administered to a human subject, have a half-life in the plasma of the subject that is no more than 30 hours, 25 hours, 20 hours, 15 hours, 10 hours, 9 hours, 8 hours or 7 hours.

In some embodiments, all components in the distribution of the LCMF poloxamer, when administered to a human subject, have a half-life in the plasma of the subject that is no more than 10 or 12 hours.

In some embodiments, the LCMF poloxamer is a poloxamer with a hydrophobe having a molecular weight of about 1,400 to 2,000 Da or 1,400 to 2,000 Da, and a hydrophile portion constituting approximately 70% to 90% or 70% to 90% by weight of the copolymer. In some embodiments, the molecular weight of the hydrophobe [CH(CH₃)CH₂O]_{b} is about or is 1,750 Da.

In some embodiments, the average molecular weight of the LCMF poloxamer is 7100 to 9510 Daltons, or 7680 to 9510 Daltons or 8,400-8,800 Daltons, or 8,200-8,800 Daltons.

In some embodiments, the percentage of high molecular weight components in the LCMF poloxamer greater than 13,000 Daltons constitute less than 1% of the total distribution of components; and following intravenous administration to a subject, does not result in a component with a circulating half-life greater than four-fold that of the circulating plasma half-life of the main peak.

In some embodiments, the percentage of high molecular weight components in the LCMF poloxamer greater than 13,000 Daltons constitute less than 1.5%, 1.2%, 0.9%, 0.8%, 0.7%, 0.6%, 0.5% or less of the total distribution of components.

In some embodiments, the polydispersity value of the LCMF poloxamer is less than 1.06, 1.05, 1.04 or 1.03.

In some embodiments, the poloxamer 188 comprises a LCMF with an unsaturation level of about 0.010 to 0.034 mEq/g or 0.026±0.008 mEq/g.

In some embodiments, the poloxamer 188 comprises a long circulating material free (LCMF) poloxamer 188, wherein:

the LCMF poloxamer 188 is a polyoxyethylene/polyoxypropylene copolymer that has the formula HO(CH₂CH₂O)_{a′}—[CH(CH₃)CH₂O]_{b}—(CH₂CH₂O)ₐH, wherein:

each of a and a′ is an integer such that the percentage of the hydrophile (C₂H₄O) is between approximately 60% and 90% by weight of the total molecular weight of the copolymer;

a and a′ are the same or different;

b is an integer such that the molecular weight of the hydrophobe [CH(CH₃)CH₂O]_{b} is between approximately 1,300 and 2,300 Daltons;

no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons;

the polydispersity value of the copolymer is less than approximately 1.07 or less than 1.07; and

following intravenous administration to a subject, the circulating plasma half-life of any components not comprising the main peak is more than 5.0-fold longer than the circulating half-life of the main peak.

In some embodiments, the poloxamer 188 comprises a long circulating material free (LCMF) poloxamer 188, wherein:

the LCMF poloxamer 188 is a polyoxyethylene/polyoxypropylene copolymer that has the formula HO(CH₂CH₂O)_{a′}—[CH(CH₃)CH₂O]_{b}—(CH₂CH₂O)ₐH;

each of a and a′ is an integer such that the percentage of the hydrophile (C₂H₄O) is between approximately 60% and 90% by weight of the total molecular weight of the copolymer;

a and a′ are the same or different;

b is an integer such that the molecular weight of the hydrophobe [CH(CH₃)CH₂O]_{b} is between approximately 1,300 and 2,300 Daltons;

no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons;

the polydispersity value of the copolymer is less than approximately 1.07 or less than 1.07; and

the LCMF copolymer is more hydrophilic than purified poloxamer 188 that contains the long circulating material (LCM).

In some embodiments, the poloxamer 188 comprises a long circulating material free (LCMF) poloxamer 188, wherein:

the LCMF poloxamer 188 is a polyoxyethylene/polyoxypropylene copolymer that has the formula HO(CH₂CH₂O)_{a′}—[CH(CH₃)CH₂O]_{b}—(CH₂CH₂O)ₐH;

each of a and a′ is an integer such that the percentage of the hydrophile (C₂H₄O) is between approximately 60% and 90% by weight of the total molecular weight of the copolymer;

a and a′ are the same or different; b is an integer such that the molecular weight of the hydrophobe [CH(CH₃)CH₂O]_{b} is between approximately 1,300 and 2,300 Daltons;

no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons;

the polydispersity value of the copolymer is less than approximately 1.07 or less than 1.07; and

the LCMF has a mean retention time (t_{R}) as assessed by reverse phase-high performance liquid chromatography that is shorter than purified poloxamer 188 In some embodiments, the mean t_{R} of the LCMF poloxamer is about or is 8.7-8.8, and that of the LCM-containing poloxamer 188 is about or is 9.9-10; and the RP-HPLC chromatography conditions are as follows (Table 1):

TABLE 1
LCMF retention time

| Column | Xterra RP18, 3.5 um, 4.6 × 100 mm | |
|---|---|---|
| Mobile Phase | A: 0.1% HOAc in Water | |
| | B: Acetonitrile | |
| Gradient | Time | % B |
| | 0 | 50 |
| | 1.0 | 50 |
| | 15.0 | 90 |
| | 16.0 | 90 |
| | 16.1 | 50 |
| | 20.0 | 50 |

| | |
|---|---|
| Flow Rate | 0.50 ml/min |
| Column Temp | 40° C. |
| ELS*Detection | N₂: 0.5 liter/minute, Nebulizer: |
| | 75° C., Evaporator: 75° C. |
| Sample Preparation | Drug Product—No dilution |
| | Purified Poloxamer 188, 150 mg/mL |
| | in 10 mM Sodium Citrate pH 6 |
| Injection Volume | 10 μL |

*ELS = evaporative light scattering

In some embodiments, the poloxamer 188 comprises a long circulating material free (LCMF) poloxamer 188, wherein:

the LCMF poloxamer 188 is a polyoxyethylene/polyoxypropylene copolymer that has the formula HO(CH₂CH₂O)_{a′}—[CH(CH₃)CH₂O]_{b}—(CH₂CH₂O)ₐH, wherein:

each of a and a′ is an integer such that the percentage of the hydrophile (C₂H₄O) is between approximately 60% and 90% by weight of the total molecular weight of the copolymer;

a and a′ are the same or different; b is an integer such that the molecular weight of the hydrophobe [CH(CH₃)CH₂O]_{b} is between approximately 1,300 and 2,300 Daltons;

no more than 1.5% of the total components in the distribution of the co-polymer are low molecular weight components having an average molecular weight of less than 4,500 Daltons;

no more than 1.5% of the total components in the distribution of the co-polymer are high molecular weight components having an average molecular weight of greater than 13,000 Daltons;

the polydispersity value of the copolymer is less than approximately 1.07 or less than 1.07; and

the capacity factor (k′) as assessed by RP-HPLC is less than the k′ for purified LCM-containing poloxamer 188.

In some embodiments, the mean k′ of the LCMF poloxamer is about or is 3.2-3.3, and that of the LCM-containing poloxamer 188 is about or is 3.6-3.7.

In some embodiments, the poloxamer 188 is produced by a method comprising:

admixing a solution of poloxamer 188 in a first alkanol with an extraction solvent comprising a second alkanol and supercritical carbon dioxide under a temperature and pressure to maintain the supercritical carbon dioxide for a first defined period, wherein:
the temperature is above the critical temperature of carbon dioxide but is no more than 40° C.;
the pressure is 220 bars to 280 bars; and
the alkanol is provided at an alkanol concentration that is 7% to 8%, such as about 7.4%, by weight of the total extraction solvent; and

increasing the concentration of the second alkanol in the extraction solvent a plurality of times in gradient steps over time of the extraction method, wherein:

each plurality of times occurs for a further defined period; and

in each successive step, the alkanol concentration is increased 1-2% compared to the previous concentration of the second alkanol; and

removing the extraction solvent from the extractor vessel to thereby remove the extracted material from the poloxamer preparation.

In some embodiments, the ratio of poloxamer to first alkanol, by weight is about or is from 2:1 to 3:1, inclusive.

In some embodiments, the plurality of times occurs in two, three, four or five gradient steps.

In some embodiments, increasing the concentration of the second alkanol in the extraction solvent occurs in two steps comprising:

i) increasing the concentration of the second alkanol from about 7% to 8% to about 8.2% to 9.5%, such as about 9.1%, for a second defined period; and

ii) increasing the concentration of the second alkanol from about 8.2% to 9.5% to about 9.6% to 11.5%, such as about 10.7%, for a third defined period.

In some embodiments, the first and second alkanol are each independently selected from among methanol, ethanol, propanol, butanol, pentanol and a combination thereof.

In some embodiments, the first and second alkanol are the same or different.

In some embodiments, the first alkanol is methanol.

In some embodiments, the second alkanol is methanol.

In some embodiments, the first alkanol is methanol and the second alkanol is methanol.

In some embodiments, the poloxamer 188 is present at a concentration of at least 10.0 mg/mL, at least 20 mg/mL, at least 30 mg/mL, at least 40 mg/mL, at least 50 mg/mL, at least 60 mg/mL, at least 70 mg/mL, at least 80 mg/mL, at least 90 mg/mL, at least 100 mg/mL, at least 115 mg/mL, at least 130 mg/mL, at least 150 mg/mL, at least 200 mg/mL or at least 225 mg/mL.

In some embodiments, the poloxamer 188 is present at a concentration of no more than 225 mg/mL.

In some embodiments, the poloxamer 188 is present at a concentration of from or from about 150 mg/mL to 225 mg/mL.

In some embodiments, the poloxamer 188 is present at a concentration of at least 15%, at least 20%, at least 25%, 10% to 25%, 22.5%, 30%, 40%, 50%, 10% to 20%, 10% to 50%, 15% to 20%, 15%-30%, 15% to 28%, 20-23%, 15% to 25%, or 20-25%.

In some embodiments, the poloxamer 188 is present at a concentration of from about 10 to about 30%.

In some embodiments, the poloxamer 188 is present at a concentration greater than about 15% up to about 30%.

In some embodiments, the poloxamer 188 is present at a concentration of from about 15 to about 25% w/v.

In some embodiments, the poloxamer 188 is present at a concentration greater than about 15% up to about 25%.

In some embodiments, the poloxamer 188 is present at a concentration of greater than 15% w/v.

In some embodiments, the poloxmer 188 is present at a concentration of from about 20% to about 25% w/v.

In some embodiments, the poloxamer 188 is present at a concentration of about 20% w/v.

In some embodiments, the poloxamer 188 is present at a concentration of about 22.5% w/v.

In some embodiments, the poloxamer 188 is present at a concentration of about 25% w/v.

In some embodiments, a sodium free solution of poloxamer 188 can be diluted to a lower concentration before use, for example, without limitation a 25% solution can be diluted to a 15% solution or a 22.5% solution can be diluted to a 15% solution.

In some embodiments, the solutions/formulations described herein contain magnesium chloride (MgCl₂). Magnesium chloride is a possible viscosity reducer (see U.S. Pat. No. 7,758,860), stabilizer (see, U.S. Publication No. 2012/0245230), and tonicity adjusting agent (see, U.S. Pat. No. 9,364,564).

In some embodiments, the solution further comprises a tonicity agent.

In some embodiments, the tonicity agent is reduced or substantially free of sodium.

In some embodiments, the tonicity agent is chosen from glucose, glycerin (glycerol), dextrose, sucrose, xylitol, fructose, mannitol, sorbitol, mannose, potassium salts, calcium salts, and magnesium salts. In some embodiments, the tonicity agent is a magnesium salt. In some embodiments, the magnesium salt is chosen from magnesium acetate, magnesium aluminate, magnesium borate, magnesium bicarbonate, magnesium carbonate, magnesium chloride, magnesium citrate, magnesium gluconate, magnesium hydroxide, magnesium lactate, magnesium metasilicate aluminate, magnesium oxide, magnesium phthalate, magnesium phosphate, magnesium silicate, magnesium stearate, magnesium succinate, magnesium tartrate, and mixtures thereof. In some embodiments, the magnesium salt is magnesium chloride. In some embodiments, the magnesium chloride is magnesium chloride hexahydrate.

In some embodiments, the concentration of the tonicity agent is from about 0 mM to about 20 mM, or about 1 mM to about 20 mM. In some embodiments, the concentration of the tonicity agent is from about 1 to about 10 mM. In some embodiments, the concentration of the tonicity agent is from about 1 to about 5 mM. In some embodiments, the concentration of the tonicity agent is from about 1 to about 2 mM.

In some embodiments, the solution further comprises an antioxidant.

In some embodiments, the solution further comprises an antioxidant that is reduced or substantially free of sodium.

In some embodiments, the antioxidant is chosen from cysteine, citric acid, dextrose, dithiothreitol, histidine, malic acid, mannitol, methionine, metabisulfate, ascorbic acid, and tartaric acid. In some embodiments, the antioxidant is citric acid.

In some embodiments, the concentration of the antioxidant is from about 0.001% to about 2%. In some embodiments, the concentration of the antioxidant is from about 0.1 mM to about 10 mM.

In some embodiments, the solution has a pH of from about 6 to about 8.

In some embodiments, the solution further comprises a buffer.

In some embodiments, the solution further comprises a buffer that is reduced or substantially free of sodium.

In some embodiments, the buffer is chosen from citrate buffer (pH about 2); citrate buffer (pH about 5); citrate buffer (pH about 6.3); phosphate buffer (pH about 7.2); phosphate buffer (pH about 9); borate buffer (pH about 9); borate buffer (pH about 10); succinate buffer (pH about 5.6); histidine buffer (pH about 6.1); carbonate buffer (pH about 6.3); acetate buffer (pH about 7.2), meglumine, and combinations thereof. In some embodiments, the buffer comprises citric acid and meglumine at an adjusted pH of about 6.

In some embodiments, the solution further comprises a pH adjusting agent.

In some embodiments, the solution further comprises a pH adjusting agent that is reduced or substantially free of sodium.

In some embodiments, the pH adjusting agent is chosen from aqueous HCl, ammonium hydroxide, meglumine, or other non-sodium buffers and components, and mixtures thereof.

Also provided is a sterile, injectable solution comprising: poloxamer 188 at a concentration of about 225 mg/mL, magnesium chloride hexahydrate at a concentration of about 0.610 mg/mL, and water for injection, wherein the sterile, injectable solution is reduced or substantially sodium-free; the poloxamer 188 is stable from oxidative decomposition; and the sterile, injectable solution has a pH of from about 4 to about 8.

In some embodiments, the osmolality of the solution is between about 100 and about 2000 mOSm/kg. In some embodiments, the osmolality of the solution is between about 300 and about 1500 mOSm/kg, or 270 to about 1500 mOSm/kg, or about 280 to about 1500 mOSm/kg. In some embodiments, the osmolality of the solution is between about 300 and about 500 mOSm/kg, between about 350 and about 500 mOSm/kg, between about 350 and about 700 mOSm/kg, between about 300 to about 700 mOSm/kg, or about 270 and about 500 mOSm/kg.

In some embodiments, the solutions further comprise one or more other active ingredients. In some embodiments, the one or more other active ingredients are chosen from acetaminophen, hydroxyurea, adenosine, amiodarone HCl, atropine sulfate, bumetanide, cefazolin, chlorothiazide sodium, dexamethasone sodium phosphate, digoxin, HCl, dobutamine diphenhydramine HCl, dopamine HCl, enalapril maleate, epinephrine HCl, fentanyl citrate, furosemide, gentamicin sulfate, heparin sodium, hydrocortisone sodium succinate, isoproterenol HCl, labetalol HCl, lidocaine HCl, mannitol, meperidine HCl, metoprolol tartrate, milrinone, nafcillin sodium, naloxone, nesiritide, norepinephrine bitartrate, ondansetron HCl, phenylephrine HCl, promethazine HCl, quinidine gluconate, and verapamil.

In some embodiments, the solution is packaged in a sealed, pharmaceutically acceptable container. In some embodiments, the atmosphere within said sealed, pharmaceutically acceptable container comprises an inert gas or atmosphere such as without limitation, argon, nitrogen, and/or carbon dioxide, or the solution container is sealed under vacuum.

In some embodiments, the dissolved oxygen in the solution is no more than about 2.0 mg/L. In some embodiments, the solution comprises no more than about 2.0 mg/L of dissolved oxygen and does not comprise an antioxidant.

In some embodiments, the sealed, pharmaceutically acceptable container is a vial. In some embodiments, the vial comprises type 1 flint glass or borosilicate glass. In some embodiments, the vial is a 100 mL vial. In some embodiments, the vial is a 500 mL vial. In some embodiments the vial is about a 10 ml to about 600 ml vial.

In some embodiments, the sealed, pharmaceutically acceptable container is an infusion bag. In some embodiments, the infusion bag comprises polyvinyl chloride (PVC), PVC with di(2-ethylhexyl)phthalate (DEHP), PVC with (tris (2-ethylhexyl) trimellitate) (TOTM), polyolefin, polypropylene or ethylene-vinyl acetate (EVA).

In some embodiments, the sealed, pharmaceutically acceptable container is sealed in a foil pouch. In some embodiments, the atmosphere within the sealed foil pouch containing the pharmaceutically acceptable container comprises argon, nitrogen, and/or carbon dioxide or inert atmosphere.

In some embodiments, the solution is not diluted prior to administration.

In some embodiments, the solution is diluted prior to administration.

In some embodiments, the solution is diluted into a solution that has reduced sodium and/or is substantially sodium free.

In some embodiments, the solution is diluted into D5W-dextrose 5% in water.

Certain polyoxyethylene/polyoxypropylene copolymers, including poloxamer 188, have beneficial biological effects on several disorders when administered to a human or animal. These activities have been described, for example in numerous publications and patent (see, e.g., U.S. Pat. Nos. 4,801,452, 4,837,014, 4,873,083, 4,879,109, 4,897,263, 4,937,070, 4,997,644, 5,017,370, 5,028,599, 5,030,448, 5,032,394, 5,039,520, 5,041,288, 5,047,236, 5,064,643, 5,071,649, 5,078,995, 5,080,894, 5,089,260, RE 36,665 (Reissue of 5,523,492), 5,605,687, 5,696,298 6,359,014, 6,747,064, 8,372,387, 8,580,245, U.S. Patent Publication Nos. 2011/0044935, 2011/0212047, 2013/0177524, and International Applications WO2006/037031 (filed as PCT/US2005/034790), WO2009/023177 (filed as PCT/US2005/037157) and WO2006/091941 (filed as PCT/US2006/006862), and PCT/US2014/45627.

Accordingly, the solution described herein can be used in a wide variety of applications, including cytoprotective, hemorheologic, anti-inflammatory, antithrombotic/pro-fibrinolytic applications, with clinical utility in diverse diseases including but not limited to acute coronary syndromes, limb ischemia, shock, stroke, heart failure, including without limitation, systolic, diastolic, congestive, and cardiomyopathies, coronary artery disease, muscular dystrophy, circulatory diseases, pathologic hydrophobic interactions in blood, preservation of organs for transplant, inflammation, sickle cell disease, such as venous occlusive crisis, and acute chest syndrome, inflammation, pain, neurodegenerative diseases, macular degeneration, thrombosis, kidney failure, burns, spinal cord injuries, ischemic/reperfusion injury, myocardial infarction, preventing or treating storage lesion in stored blood and blood products, improving the safety and efficacy of stored blood for use in transfusions, hemo-concentration, amyloid oligomer toxicity, diabetic retinopathy, diabetic peripheral vascular disease, sudden hearing loss, peripheral vascular disease, cerebral ischemia, transient ischemic attacks, critical limb ischemia, respiratory distress syndrome (RDS), and adult respiratory distress syndrome (ARDS). The solutions provided herein can be used to treat any disease or condition in which P188 has previously been used or is known to be effective.

Also provided is a method of treating a disease or condition in a subject, comprising administering the solution described herein, wherein the disease or condition is selected from acute coronary syndromes, limb ischemia, shock, stroke, heart failure, sickle cell disease, neurodegenerative diseases, macular degeneration, thrombosis and ARDS, kidney failure, liver disease, sickle cell disease and associated venous occlusive crisis, and acute chest syndrome.

Also provided is a method of treating shock in a subject, comprising administering the solution described herein. In some embodiments, shock is septic shock, hypovolumic shock or distributive shock.

Also provided is a method of treating limb ischemia in a subject, comprising administering the solution described herein. In some embodiments, limb ischemia is peripheral limb ischemia or acute limb ischemia.

Also provided is a method of treating stroke in a subject, comprising administering the solution described herein. In some embodiments, stroke is vasospastic stroke, thrombotic stroke, hemorrhagic stroke, or ischemic stroke.

Also provided is a method of treating heart failure in a subject, comprising administering the solution described herein. In some embodiments, heart failure is acute heart failure, chronic heart failure, systolic heart failure (heart failure with reduced ejection fraction), or diastolic heart failure (heart failure with preserved ejection fraction).

In some embodiments, the heart failure is manifested by the presence of one or more of arrhythmias, elevated blood pressure, narrowing arteries, catheterization or altered cardiac output.

In some embodiments, the heart failure is systolic heart failure. In some embodiments, systolic heart failure is manifested by reduced left ventricular (LV) ejection fraction (EF), increased LV end-systolic volume, left ventricular hypertrophy or elevated LV end-systolic pressure.

In some embodiments, the heart failure is diastolic heart failure. In some embodiments, the diastolic heart failure is manifested by increased myocardial mass with normal left ventricular chamber size or elevated LV end-diastolic pressure.

Also provided is a method of treating sickle cell disease or symptoms thereof in a subject, comprising administering the solution described herein. In some embodiments, sickle cell disease or symptoms thereof includes acute vaso-occlusive crisis, acute chest syndrome, splenic sequestration and/or priapism.

Also provided is a method of treating thrombosis in a subject, comprising administering the solution described herein. In some embodiments, thrombosis is arterial thrombosis or venous thrombosis.

Also provided is a method of treating macular degeneration in a subject, comprising administering the solution described herein. In some embodiments, macular degeneration includes dry AMD and wet AMD.

Also provided is a method of treating acute coronary syndromes in a subject, comprising administering the solution described herein. In some embodiments, acute coronary syndromes include acute myocardial infarction and unstable angina.

In some embodiments, the solutions described herein are administered with one or more other active ingredients. In some embodiments, the one or more other active ingredients are chosen from acetaminophen, adenosine, amiodarone HCl, atropine sulfate, bumetanide, cefazolin, chlorothiazide sodium, hydroxyurea, dexamethasone sodium phosphate, digoxin, HCl, dobutamine diphenhydramine HCl, dopamine HCl, enalapril maleate, epinephrine HCl, fentanyl citrate, furosemide, gentamicin sulfate, heparin sodium, hydrocortisone sodium succinate, isoproterenol HCl, labetalol HCl, lidocaine HCl, mannitol, meperidine HCl, metoprolol tartrate, milrinone, nafcillin sodium, naloxone, nesiritide, norepinephrine bitartrate, ondansetron HCl, phenylephrine HCl, promethazine HCl, quinidine gluconate, and verapamil.

The skilled physician or pharmacist or other skilled person, can select appropriate concentrations and administration conditions for the particular subject, condition treated and target circulating concentration. If necessary, a particular dosage and duration and treatment protocol can be empirically determined or extrapolated. Dosages for poloxamer 188 previously administered to human subjects and used in clinical trials can be used as guidance for determining dosages for poloxamer 188, such as a purified poloxamer 188 described herein. Dosages for poloxamer 188 can also be determined or extrapolated from relevant animal studies. Factors such as the level of activity and half-life of poloxamer 188 can be used in making such determinations. Particular dosages and regimens can be empirically determined based on a variety of factors. Such factors include body weight of the individual, general health, age, the activity of the specific compound employed, sex, diet, time of administration, rate of excretion, drug combination, the severity and course of the disease, the patient's disposition to the disease, and the judgment of the treating physician.

In some embodiments, the poloxamer, such as P188 (e.g., LCMF P188), is formulated for administration to a patient at a dosage of about 100 mg/kg to 2,000 mg/kg depending on the condition to be treated.

The dose of poloxamer is administered at a concentration and in a fluid volume that suits the mode of administration and the physiological needs of the patient. Generally, for longer term infusions (such as a 12, 24, or 48 hour continuous infusion) the volume administered is typically not greater than about 5.0 mL/kg/hr. such as 4.5 ml/kg/hr, 4.0 ml/kg/hr, 3.5 ml/kg/hr, 3.0 ml/kg/hr, 2.5 ml/kg/hr, 2.0 ml/kg/hr, 1.5 ml/kg/hr, 1.0 ml/kg/hr, 0.5 ml/kg/hr, 0.25 ml/kg/hr or 0.125 ml/kg/hr. For shorter term administrations (such as bolus administrations or short term infusions) the dose of poloxamer may be administered in a volume greater than 5.0 ml/kg/hr such as 7.5 ml/kg/hr or 10.0 ml/kg/hr or 12.5 ml/kg/hr or 15 ml/kg/hr or even higher depending upon the needs of the patient. The poloxamer can be administered as a single dose or in multiple doses that are repeated over various intervals, such as hourly, daily, weekly, monthly or more. For infusions, the infusions can provide the appropriate dosage to the subject over a time period that is typically 1 hour to 72 hours, such as 12 hours, 24 hours or 48 hours. In some embodiments, administration of the poloxamer can include a titration dose, such as 25-100 mg/kg for about 0.5 hours-2 hours, or about 1-2 hours, or about 1 hour.

In some embodiments, the poloxamer 188 is formulated for administration to a patient at a dosage of about 100 to 600 mg/kg patient body weight, such as 100 to 500 mg/kg patient body weight, for example 100 mg/kg to 450 mg/kg, 100 to 400 mg/kg, 100 mg/kg to 300 mg/kg, 100 mg/kg to 200 mg/kg, 200 mg/kg to 500 mg/kg, 200 mg/kg to 450 mg/kg, 200 mg/kg to 400 mg/kg, 200 mg/kg to 300 mg/kg, 300 mg/kg to 500 mg/kg, 300 mg/kg to 450 mg/kg 300 mg/kg to 400 mg/kg, 400 mg/kg to 500 mg/kg, 400 mg/kg to 450 mg/kg or 450 mg/kg to 500 mg/kg patient body weight, such as about 100, 125, 150, 200, 250, 300, 350, 400, 450, 500, or 600 mg/kg patient body weight. In some embodiments, the poloxamer is formulated for administration at a dosage of about 200-450 mg/kg, such as 400 mg/kg patient body weight.

In some embodiments, the volume to be administered is not greater than 4.0 mL/kg of a subject. For example, the volume in which the dose is administered to a subject can be 0.4 mL/kg to 4.0 mg/kg, 0.4 mL/kg to 3.5 mL/kg, 0.4-3.0 ml/kg, 0.4-2.5 ml/kg, 0.4 mL/kg to 2.0 mL/kg, 0.4 mL/kg to 1.8 mL/kg. 0.4 mL/kg to 1.4 mL/kg, 0.4 mL/kg to 1.0 mL/kg, 0.4 mL/kg to 0.6 mL/kg, 0.6 mL/kg to 4.0 mL/kg, 0.6 mL/kg to 3.0 mL/kg, 0.6 mL/kg to 2.0 mL/kg, 0.6 mL/kg to 1.8 mL/kg, 0.6 mL/kg to 1.4 mL/kg, 0.6 mL/kg to 1.0 mL/kg, 1 mL/kg to 4 mL/kg, 1.0 ml/kg-3.0 ml/kg, 1 mL/kg to 2.5 mL/kg, 1 mL/kg to 2.0 mL/kg, 1 mL/kg to 1.8 mL/kg, 1 mL/kg to 1.4 mL/kg, 1.4 mL/kg to 4.0 mL/kg, 1.4 ml/kg-3.0 ml/kg, 1.4 mL/kg to 2.5 mL/kg, 1.4 mL/kg to 2.0 mL/kg, 1.4 mL/kg to 1.8 mL/kg, 1.8 ml/kg-4.0 ml/kg, 1.8 mL/kg to 3.0 mL/kg, 1.8 mL/kg to 2.5 mL/kg, 1.8 mL/kg to 2.0 mL/kg, 2.0 ml/kg-4.0 ml/kg, 2.0 mL/kg to 3.0 mL/kg, 2.0 mL/kg to 2.5 mL/kg or 2.5 mL/kg to 3.0 mL/kg. For example, a composition with a concentration of 22.5% (i.e. 225 mg/mL) that is administered to a 100 kg subject at a dose of 100 mg/kg would require a volume of about 44 ml or about 0.4 mL/kg to achieve that dose.

In some embodiments, the dose is administered as an infusion. Generally the infusion is an intravenous infusion. The infusion, to provide the appropriate dosage, can be provided to the subject over a time period that is 1 hour to 24 hours, 1 hour to 12 hours, 1 hour to 6 hours, 1 hour to 3 hours, 1 hour to 2 hours, 2 hours to 24 hours, 2 hours to 12 hours, 2 hours to 6 hours, 2 hours to 3 hours, 3 hours to 24 hours, 3 hours to 12 hours, 3 hours to 6 hours, 6 hours to 24 hours, 6 hours to 12 hours, or 12 hours to 24 hours, such as generally over at time period that is up to or is about 1 hour, 2 hours, 3 hours, 4 hours, 5 hours, 6 hours, 7 hours, 8 hours, 9 hours, 10 hours, 12 hours, 15 hours, 18 hours, 20 hours, 22 hours or more. It is within the level of a treating physician to determine the appropriate time and rate of infusion to deliver an effective dose that can be tolerated by a subject.

In some embodiments, the infusion of poloxamer 188 is provided as a single infusion that is not repeated for at least a week, and then can be subsequently repeated at intervals of at least a week, generally up to 2-4 weeks, and, as improvement is observed, increasingly longer intervals, and/or lower dosages.

In some embodiments, the administration can be repeated once every week, once every 2 weeks, once every three weeks, once every 4 weeks, once every 5 weeks or once every 6 weeks. For example, the dose can be repeated between 1 week to 4 weeks after the previous dose, such that the dose is repeated at 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days 26 days, 27 days, 28 days, 29 days, 30, 31 days, 32 days, 33 days, 34 days, 35 days, 36 days, 37 days, 38 days, 39 days, 40 days, 41 days, or 42 days following completion of the prior dose. The dose that is administered in the repeated dosing can be the same or different than the prior dose. For example, it can be increased or decreased from the prior dose. It is within the level of the treating physician to determine the appropriate frequency of administration and level or amount of dosages in repeated dosings.

The length of time of the cycle of administration can be empirically determined, and is dependent on the disease to be treated, the severity of the disease, the particular patient, and other considerations within the level of skill of the treating physician. The length of time of treatment can be one day, one week, two weeks, one months, several months, one year, several years or more. Over that time, the poloxamer 188 can be administered no more than once weekly, such every 7 days, 8 days, 9 days, 10 days, 11 days, 12 days, 13 days, 14 days, 15 days, 16 days, 17 days, 18 days, 19 days 20 days, 21 days, 22 days, 23 days, 24 days, 25 days, 26 days, 27 days or 28 days or more as described above. If disease symptoms persist in the absence of discontinued treatment, treatment can be continued for an additional length of time. Over the course of treatment, evidence of disease and/or treatment-related toxicity or side effects can be monitored.

In addition, the cycle of administration can be tailored to add periods of discontinued treatment in order to provide a rest period from exposure to the treatment. The length of time for the discontinuation of treatment can be for a predetermined time or can be empirically determined depending on how the patient is responding or depending on observed side effects. For example, the treatment can be discontinued for one week, two weeks, one month or several months.

### EXAMPLES

The following examples are included for illustrative purposes only and are not intended to limit the scope of the inventions herein.

### Example 1

Preparation of Sterile, Injectable Sodium-Free Solution

A solution of 225 mg/mL LCMF poloxamer 188 with 10 mM citric acid, and 3 mM magnesium chloride with pH adjusted with 24 mM meglumine was prepared using the following procedure (Table 2).

TABLE 2
Preparation of 22.5% sodium-free LCMF injectable solution

| | | | Amount | Amount | Amount |
|---|---|---|---|---|---|
| | | Compendial | per mL | per L | per kg |
| Component | Function | Grade | (mg/mL) | (g/L) | (g/kg) |
| LCMF | Drug | Manufacturer's | 225 | 225 | 217 |
| Poloxamer | substance | Specification | | | |
| 188 | | | | | |
| Citric acid, | Buffer | USP/Ph. Eur. | 1.92 | 1.92 | 1.85 |
| anhydrous | | | | | |
| Magnesium | Tonicity | USP/Ph. Eur. | 0.61 | 0.61 | 0.59 |
| chloride | Agent/ | | | | |
| hexa- | viscosity | | | | |
| hydrate | reducing | | | | |
| | agent, | | | | |
| | stabilizer; | | | | |
| Meglumine | pH | USP/Ph. Eur | 4.69 | 4.69 | 4.53 |
| | adjustment | | | | |
| Water for | Solvent | USP/Ph. Eur. | QS to | QS to | 776 |
| injection | | | 1.0353 | 1.0353 | |
| | | | g | kg | |
| Nitrogen | Inert | NF/Ph. | QS for | QS for | QS for |
| | atmosphere | Eur./JP | process | process | process |
| 1N HCl | pH | NF/Ph. Eur. | As | As | As |
| | adjustment | | needed | needed | needed |

1. Using a stainless steel tube and frit, sparge at least 1200 g of vigorously for at least 10 minutes until the dissolved oxygen is no more than 2 mg/L.
2. Transfer ˜700 g of SWFI to a tarred 2-L stainless steel vessel
3. Position the overhead mixer over the vessel and set to 250 RPM, or higher, until a vortex is observed.
4. Slowly add 1.92 g of citric acid, anhydrous into ˜700 g of SWFI with stirring. Mix until a homogenous solution is obtained as determined by visual inspection.
5. Slowly add 0.61 g magnesium chloride hexahydrate (MgCl₂.6H₂O) to the vessel with stirring, and mix until a homogenous solution is obtained as determined by visual inspection.
6. Slowly add 4.69 g of meglumine to the vessel and mix until a homogenous solution is obtained as determined by visual inspection.
7. Slowly and portion-wise (to avoid foaming) add 225 g of poloxamer 188 drug substance to the vessel and mix until a homogenous solution is obtained (overnight, if necessary).
8. Nitrogen purged SWFI is added to the vessel to reach 1035.3 g or 100% target assay

Using the method of Example 20, the salt concentration of this formulation was found to be 0.0429 μg/mL

### Example 2

Preparation of Sterile, Injectable Sodium-Free Solution

A solution of 150 mg/mL LCMF poloxamer 188 was prepared using the following components as described above (Table 3).

TABLE 3
Preparation of 15% sodium free LCMF poloxamer-
188 injectable solution

| | | | Amount per |
|---|---|---|---|
| | | Amount | 104 mL vial |
| | | per mL | fill volume |
| Component | Function | (mg/mL) | (mg) |
| LCMF | Drug | 150 | 15600 |
| Poloxamer 188 | Substance | | |
| Citric acid, | Buffer | 1.92 | 199.8 |
| Anhydrous | | | |
| Ammonium | pH adjustment | 1.97 | 204.9 |
| hydroxide | | | |
| Magnesium | Tonicity Agent; | 7.18 | 746.7 |
| Chloride | viscosity | | |
| Hexahydrate | reducing agent, | | |
| | stabilizer | | |
| Hydrochloric | pH | QS for | QS for |
| Acid | adjustment | process | process |
| Water for | Diluent | QS to | QS to |
| injection | | 1.024 g | 106.5 g |
| Nitrogen | Inert | QS for | QS for |
| | atmosphere | process | process |

### Example 3

Preparation of Sterile, Injectable Sodium-Containing Solution for Use as a Comparator in the Studies Herein

A solution of 150 mg/mL LCMF poloxamer 188 with a total sodium content of 0.45% sodium chloride (half-normal saline) was prepared using the following components as described above in Example 1 and shown in (Table 4).

TABLE 4
Preparation of half-normal sodium-containing solution of 15%
LCMF poloxamer-188

| | | | | Amount per |
|---|---|---|---|---|
| | | | Amount | 104 mL Vial |
| | | Compendial | per mL | Fill Volume |
| Component | Function | Grade | (mg/mL) | (mg) |
| LCMF | Drug | Manufacturer's | 150 | 15600 |
| poloxamer | substance | Specification | | |
| 188 | | | | |
| Citric acid, | Buffer | USP/Ph. Eur. | 0.366 | 38.06 |
| anhydrous | | | | |
| Trisodium | Buffer | USP/Ph. Eur. | 2.38 | 247.52 |
| citrate, | | | | |
| dihydrate | | | | |
| Sodium | Isotonicity | USP/Ph. Eur. | 3.08 | 320.32 |
| chloride | adjuster | | | |
| Water for | Diluent | USP/Ph. Eur. | 1.0 mL | QS to |
| injection | | | | 104 mL |
| Nitrogen | Inert | NF/Ph. Eur. | QS for | QS for |
| | atmosphere | | process | process |
| 1N HCl | pH adjustment | NF/Ph. Eur. | As needed | As needed |
| 1N NaOH | pH adjustment | NF/Ph. Eur. | As needed | As needed |

USP = United States Pharmacopeia;
Ph. Eur. = European Pharmacopoeia;
QS = Quantity sufficient;
NF = National Formulary;
N = Normal;
HCl = Hydrochloric acid;
NaOH = Sodium hydroxide.

### Example 4

Particle Studies for Various Formulations of 15% Sodium Free LCMF Poloxamer Injectable Solutions

Particles were observed in a batch of substantially sodium-free 15% poloxamer-188 LCMF. The interactions of various components of the drug product were evaluated to determine the cause of the particle formation. Table 5 shows the compositions of the nine formulations tested.

TABLE 5
Particle studies of various formulations of 15% LCMF poloxamer
injectable solutions

| | Mg | Citrate | Ammonium | Sodium | Hydroxide | Chloride |
|---|---|---|---|---|---|---|
| Formulation | (mMol) | (mMol) | (mMol) | (mMol) | (mMol) | (mMol) |
| 1: Sodium Free Formulation | 234.46 | ~10 | 34.10 | N/A | 34.10 | 468.9 |
| 2: 50% Reduction in MgCl₂ Content | 117.60 | ~10 | 34.10 | N/A | 34.10 | 235.19 |
| 3: Excess Citrate Ammonium MgCl₂ | 972.28 | 517.38 | 507.38 | N/A | N/A | 1943.62 |
| 4: Excess Citrate Ammonium | N/A | 624.25 | 614.25 | N/A | N/A | N/A |
| 5: Excess NH₄OH | N/A | ~10 | 175.39 | N/A | 175.39 | N/A |
| 6: Excess Magnesium Citrate | 62.78 | 335.53 | N/A | N/A | N/A | N/A |
| 7: Excess Citrate NH₄OH | N/A | 335.53 | 175.39 | N/A | 175.39 | N/A |
| 8: Excess Sodium Citrate | N/A | 335.53 | N/A | 1006.7 | N/A | N/A |
| 9: Excess MgCl₂ | 972.28 | N/A | N/A | N/A | N/A | 1943.62 |

Appearance, pH and D.O. were measured for each sample as shown in Table 6.

TABLE 6
appearance, pH and D.O. for
formulations made in Table 5

| Formulation | pH | DO | Appearance |
|---|---|---|---|
| 1 | 6.07 | 1.96 | Clear, colorless |
| | | | solution |
| 2 | 3.51 | 1.70 | Light yellow, |
| | | | clear solution |
| 3 | 3.22 | 1.70 | Initially Light |
| | | | yellow, clear |
| | | | solution; |
| | | | particles formed |
| | | | after storage |
| | | | at 2-8° C. |
| 4 | 4.99 | 1.68 | Light yellow, |
| | | | clear solution |
| 5 | 9.58 | 0.75 | Clear, colorless |
| | | | solution |
| 6 | N/A | N/A | Solid white, |
| | | | pasty material |
| 7 | 2.37 | N/A | Clear, colorless |
| | | | solution |
| 8 | 7.53(bottom), | N/A | Two distinct |
| | 7.68(top) | | layers, not clear, |
| | | | particles |
| 9 | 3.80 | N/A | Clear, colorless |
| | | | solution |

Based on these data, formulations 3 and 6 suggest the particle formation was due to a complexation between magnesium, citrate, and poloxamer.

### Example 5

Osmolality

The osmolality of LCMF poloxamer 188 in water was evaluated over the range 25 mg/mL to 400 mg/mL using vapor pressure osmometry (Table 7). As the concentration of poloxamer 188 in the solutions increased an exponential increase in osmolality was observed. Isotonic solutions are typically defined as possessing an osmolality value between 270-300 mOsm/kg water. Any solutions with osmolality values below 270 mOsm/kg or above 300 mOsm/kg are considered hypotonic and hypertonic respectively.

TABLE 7
osmolality of LCMF
poloxamer 188 in water

| Concentration | Osmolality |
|---|---|
| (wt/wt %) | (mOsm/kgH2O) |
| 2.5 | 6 |
| 5 | 14 |
| 10 | 53 |
| 15 | 125 |
| 20 | 241 |
| 25 | 417 |
| 30 | 682 |
| 35 | 1015 |
| 40 | 1489 |

To better understand the effect of poloxamer 188 on whole blood 13 solutions and 2 controls were evaluated via blood smears. Drug substance was added to sterile water for injection at 6 concentrations ranging from 15% to 40% and the osmolality values of the solutions were recorded. Sodium chloride was then added to sterile water for injection to make six sodium chloride solutions with osmolality values matching those of the poloxamer 188 solutions. An isotonic 15% poloxamer 188 solution was used as a comparator. Lastly, a positive and negative control were tested (Table 8).

TABLE 8
formulations used to test the effect of various
poloxamer-188 formulations on blood

| LCMF Poloxamer | NaCl | | |
|---|---|---|---|
| 188 Concentration | Concentration | | Osmolality |
| (w/w) | (w/w) | Vehicle | (mOsm/kg) |
| 15% | 0 | SWFI | 117 |
| 20% | 0 | SWFI | 237 |
| 25% | 0 | SWFI | 436 |
| 30% | 0 | SWFI | 698 |
| 35% | 0 | SWFI | 1008 |
| 40% | 0 | SWFI | 1506 |
| 0 | 0.36% | SWFI | 116 |
| 0 | 0.74% | SWFI | 224 |
| 0 | 1.35% | SWFI | 432 |
| 0 | 2.17% | SWFI | 690 |
| 0 | 3.13% | SWFI | 995 |
| 0 | 4.67% | SWFI | 1521 |
| 15% poloxamer 188 | 0.45% | 10 mM | 347 |
| formulation of Ex. 2 | | citrate buffer | |
| Positive control | 0 | SWFI | 0 |
| Negative control | 0.9% | SWFI | 308 |

SWFI = Sterile water for injection

The study revealed that there were no signs of crenated or lysed erythrocytes in the solutions containing poloxamer 188. However, crenated cells were present in all smears made with the NaCl solutions that did not contain poloxamer 188. The comparator solution (15% poloxamer solution with sodium) showed no signs of crenated cells when mixed with whole blood. This suggests that poloxamer 188 containing solutions do not behave as expected and potentially exhibit a protective effect on red blood cells in non-isotonic solutions.

The effect of poloxamer 188 on whole blood was evaluated via blood smears again using 19 pilot formulations and 2 controls. Table 9 below describes the compositions of the 19 solutions examined. As seen in the previous blood smear study, no crenation or lysis was observed for any solutions containing poloxamer 188, including solutions that are substantially sodium-free. The results of this testing confirmed the unexpected protective effect poloxamer 188 has on red blood cells that was seen in the previous blood smear study. These data suggest that isotonicity, as measured by freezing point or vapor pressure osmometry is not required for some poloxamer 188 solutions to be compatible with whole blood.

TABLE 9
Composition of Blood Smear Test Articles

| | LCMF | Citric | | | | |
|---|---|---|---|---|---|---|
| Blood | Poloxamer | Acid | MgCl₂ | NaCl | Meglumine | Crenation |
| Smear # | 188 (mg/mL) | (mM) | (mM) | (mM) | (mM) | % |
| 1 | 150 | 30 | 3 | 0 | 0 | 0 |
| 2 | 150 | 10 | 0 | 52 | 0 | 0 |
| 3 | 150 | 30 | 3 | 0 | 73 | 0 |
| 4 | 150 | 30 | 0 | 0 | 73 | 0 |
| 5 | 150 | 30 | 0 | 0 | 0 | 0 |
| 6 | 150 | 10 | 35 | 0 | 30 | 0 |
| 7 | 200 | 10 | 3 | 0 | 0 | 0 |
| 8 | 200 | 10 | 0 | 0 | 0 | 0 |
| 9 | 225 | 10 | 0 | 0 | 0 | 0 |
| 10 | 225 | 10 | 3 | 0 | 0 | 0 |
| 11 | 250 | 10 | 3 | 0 | 0 | 0 |
| 12 | 250 | 10 | 0 | 0 | 0 | 0 |
| 13 | 300 | 10 | 0 | 0 | 0 | Not |
| | | | | | | possible to |
| | | | | | | read |
| 14 | 300 | 10 | 3 | 0 | 0 | Not |
| | | | | | | possible to |
| | | | | | | read |
| 15 | 67 | 4.47 | 0 | 65.8 | 0 | 0 |
| 16 | 150 | 10 | 35 | 0 | 0 | Not |
| | | | | | | possible to |
| | | | | | | read |
| 17 | 200 | 10 | 3 | 0 | 24 | 0 |
| 18 | 225 | 10 | 3 | 0 | 24 | 0 |
| 19 | 250 | 10 | 3 | 0 | 24 | 0 |

### Example 6

Complement Activation in Various Test Solutions

The objective of this study was to quantify human serum complement factors following ex vivo activation by various poloxamer 188 test solutions, using qualified ELISA methods. The method qualifications and analysis of Bb and sC5b-9 in human serum were done using the Microvue Complement Bb Plus Immunoassay (Quidel Corporation, cat. no. A027, supplier recommended minimum required dilution (MRD) of 1/20) and the Microvue Complement SC5b-9 Plus Immunoassay (Quidel Corporation, cat. no. A020, supplier recommended MRD of 1/40) kits. Serum samples from a total of 3 healthy human donors were collected and activated with 19 test item formulations (Table 10) or control items and analyzed for Bb and sC5b-9 content. Results are shown in FIG. 3. In general solutions with higher magnesium concentrations exhibited elevated Bb content. Also solutions with higher concentrations of poloxamer 188 displayed elevated sC5b levels.

TABLE 10
Poloxamer 188 Test Solutions
(note SF indicates a sodium free formulation)

| | |
|---|---|
| 1. | (SF) 15% LCMF poloxamer 188, 10 mM citric acid, 35 mM MgCl₂ |
| 2. | (SF) 15% LCMF poloxamer 188, 30 mM citric acid |
| 3. | (SF)15% LCMF poloxamer 188, 30 mM citric acid, 3 mM MgCl₂ |
| 4. | (SF) 20% LCMF poloxamer 188, 10 mM citric acid, 3 mM MgCl₂ |
| 5. | (SF) 22.5% LCMF poloxamer 188, 10 mM citric acid, 3 mM MgCl₂ |
| 6. | (SF) 25% LCMF poloxamer 188, 10 mM citric acid, 3 mM MgCl₂ |
| 7. | (SF) 30% LCMF poloxamer 188, 10 mM citric acid, 3 mM MgCl₂ |
| 8. | (SF) 20% LCMF poloxamer 188, 10 mM citric acid |
| 9. | (SF) 22.5% LCMF poloxamer 188, 10 mM citric acid |
| 10. | (SF) 25% LCMF poloxamer 188, 10 mM citric acid |
| 11. | (SF) 30% LCMF poloxamer 188, 10 mM citric acid |
| 12. | (SF) 15% LCMF poloxamer 188, 10 mM citric acid, 35 mM MgCl₂, |
| | 30 mM meglumine |
| 13. | (SF)15% LCMF poloxamer 188, 30 mM citric acid, 3 mM MgCl₂, |
| | 73 mM meglumine |
| 14. | (SF)15% LCMF poloxamer 188, 30 mM citric acid, 73 mM |
| | meglumine |
| 15. | 6.7% LCMF poloxamer 188, 4.5 mM citric acid, 65.8 mM NaCl |
| 16. | 15% LCMF poloxamer 188, 10 mM citric acid, 52 mM NaCl |
| 17. | (SF) 20% LCMF poloxamer 188, 10 mM citric acid, 3 mM MgCl₂, |
| | meglumine |
| 18. | (SF) 22.5% LCMF poloxamer 188, 10 mM citric acid, 3 mM MgCl₂, |
| | 24 mM meglumine |
| 19. | (SF) 25% LCMF poloxamer 188, 10 mM citric acid, 3 mM MgCl₂, |
| | 24 mM meglumine |

### Example 7

pH Stability and Buffer Compatibility

The pH of a solution has a catalytic effect on the degradation rate of many drugs. To assess the role of pH and buffer species in the solution stability of LCMF poloxamer 188, the following tests were conducted:
Poloxamer 188 was evaluated in citrate, phosphate and borate buffers over the pH range 2 to 10 under accelerated conditions.
The effect of buffer species on solution stability was determined by overlapping citrate and phosphate buffers at pH 5 as well as phosphate and borate buffers at pH 9.
The effect the initial concentration of poloxamer 188 has on the degradation rate was evaluated at 20 and 300 mg/mL at pH 2, 7.2 and 10.
The compatibility of various buffer species with poloxamer 188 was determined based on the solutions ability to maintain a constant pH and potency value when stored under accelerated conditions.

All samples were analyzed for pH, appearance, osmolality, and potency using the internalized ELSD HPLC method. The results for pH and potency can be found in Table 11 below.

The solutions at high pH displayed greater stability compared to the more acidic solutions. The target pH range for a parenteral solution is typically 6 to 8. Poloxamer 188 displayed acceptable stability in this range. Buffer species showed a potential to affect stability as demonstrated by the citrate buffered solution's increased stability compared to the phosphate buffered solution at pH 5. With regards to initial concentration, it was shown that an increase in poloxamer 188 concentration does not increase the rate of degradation.

TABLE 11
pH stability and buffer compatibility

| % LCMF | | | | | | | | |
|---|---|---|---|---|---|---|---|---|
| Poloxamer | Buffer | Buffer | t = 0 | t = 1 week | t = 2 week | t = 3 week | t = 3 week | Concentration |
| 188 | (50 mM) | pH | pH | pH | pH | pH | % Potency | (mg/mL) |
| 2% | Citrate | 1.99 | 2.31 | 2.20 | — | 2.02 | 50.0 | 10 |
| | Citrate | 5.02 | 5.13 | 5.10 | — | 4.68 | 55.9 | 11.18 |
| | Phosphate | 4.95 | 5.29 | 3.68 | — | 3.22 | 40.5 | 8.1 |
| | Phosphate | 7.22 | 7.23 | 6.92 | — | 6.90 | 65.8 | 13.16 |
| | Phosphate | 9.00 | 9.06 | 9.04 | — | 8.86 | 87.8 | 17.56 |
| | Borate | 8.97 | 8.98 | 8.90 | — | 8.62 | 82.9 | 16.58 |
| | Borate | 9.98 | 9.98 | 10.01 | — | 9.82 | 95.3 | 19.06 |
| 20% | Acetate | 3.98 | 4.50 | 4.39 | 4.33 | 4.00 | 90.2 | 180.4 |
| | Tartrate | 4.38 | 4.86 | 4.62 | 4.62 | 4.46 | 89.6 | 179.2 |
| | Citrate | 4.82 | 5.24 | 5.11 | 5.05 | 4.80 | 94.7 | 189.4 |
| | Succinate | 5.59 | 5.98 | 5.85 | 5.82 | 5.64 | 94.8 | 189.6 |
| | Glycine | 5.85 | 6.89 | 4.78 | 4.33 | 4.06 | 94.8 | 189.6 |
| | Sulfate | 5.95 | 6.50 | 4.25 | 3.86 | 3.59 | 93.3 | 186.6 |
| | Histidine | 6.10 | 6.22 | 6.08 | 5.83 | 5.69 | 93.7 | 187.4 |
| | Carbonate | 6.32 | 7.12 | 7.45 | 7.53 | 7.82 | 94.2 | 188.4 |
| | Citrate | 6.39 | 6.75 | 6.63 | 6.41 | 6.22 | 95.2 | 190.4 |
| | Acetate | 7.19 | 6.86 | 6.33 | 5.94 | 5.65 | 93.8 | 187.6 |
| | Phosphate | 7.19 | 7.46 | 7.31 | 7.18 | 7.07 | 95.8 | 191.6 |
| | TRIS | 7.22 | 7.08 | 5.98 | 4.94 | 4.09 | 94.8 | 189.6 |
| 30% | Citrate | 1.99 | 2.78 | 2.67 | 2.69 | 2.67 | 96.1 | 288.3 |
| | Phosphate | 7.22 | 7.91 | 7.49 | 7.49 | 7.52 | 101.5 | 304.5 |
| | Borate | 9.98 | 10.69 | 10.29 | 10.24 | 10.05 | 101.1 | 303.3 |

The buffer species evaluated at 20% poloxamer 188 were capable of maintaining a pH value within 1 unit of the target when subjected to 3 weeks of accelerated conditions with the exception of acetate (pH 7.19), sulfate, glycine and tris. Likewise, the % potency value of poloxamer 188 was above 90% for all buffers with the exception of tartrate at 89.6%. The more acidic target pH for tartrate solution (pH 4.4) likely played a role in its instability.

### Example 8

Oxygen Content

Poloxamer 188 (purified poloxamer 188 and LCMF poloxamer 188) decomposes in the presence of oxygen. To better quantitate the effect of oxygen content on two concentrations of poloxamer 188 in SWFI was evaluated. For the study, 50 mL batches of each concentration were compounded and samples were sparged with nitrogen gas for set periods of time to achieve a target dissolved oxygen value. An aliquot of approximately 3 mL was transferred to a screw top vial that was capped with Nitrogen gas. All samples were evaluated for dissolved oxygen (D.O.) content only.

TABLE 12
oxygen effect on two concentrations of poloxamer 188 in SWFI

| % | Time of | T = 0 | T = 24 hr | T = 48 hr |
|---|---|---|---|---|
| poloxamer | Sparging | (D.O.) | (D.O.) | (D.O.) |
| 188 | (N₂) (Sec) | (mg/L) | (mg/L) | (mg/L) |
| 2 | 0 | 9.82 | 4.36 | * |
| 2 | 60 | 5.57 | 9.92 | 6.53 |
| 2 | 120 | 3.73 | 5.18 | 9.81 |
| 25 | 0 | 9.18 | 6.04 | * |
| 25 | 60 | 6.88 | 6.51 | 8.34 |
| 2 | 300 | 1.22 | 5.07 | * |
| 25 | 300 | 1.90 | 5.33 | * |

* Sample was not scheduled to be analyzed

No distinct pattern was observed in the dissolved oxygen study above. The dissolved oxygen content of the samples changed sporadically over the course of the study.

Additional samples were evaluated for volatile impurities and residual methanol after being stored at 5 and 40° C. The results of these analyses can be found in the Table 13 below. The samples displayed a strong correlation with increased oxygen content resulting in an increase in volatile impurities.

TABLE 13
samples for volatile impurities after storage at 5 and 40° C.

| % | | T = 0 | Storage | Volatile Impurities | | | |
|---|---|---|---|---|---|---|---|
| poloxamer | Target | (DO) | Temperature | Acetaldehyde | Propionaldehyde | Acetone | Methanol |
| 188 | DO | (mg/L) | (° C.) | (μg/g) | (μg/g) | (μg/g) | (μg/g) |
| 2 | 5 | 5.57 | 5 | 71 | 33 | 35 | 114 |
| 2 | 10 | 9.82 | 5 | 69 | 35 | 36 | 115 |
| 2 | <2 | 1.22 | 40 | 209 | 51 | 30 | 110 |
| 2 | 2 | 3.73 | 40 | 181 | 60 | 47 | 112 |
| 2 | 5 | 5.57 | 40 | 238 | 68 | 57 | 125 |
| 2 | 10 | 9.82 | 40 | 266 | 77 | 64 | 121 |
| 25 | 5 | 9.18 | 5 | 19 | 6 | 2 | 7 |
| 25 | 10 | 6.88 | 5 | 1 | 0 | 7 | 1 |
| 25 | <2 | 1.90 | 40 | 82 | 36 | 4 | 11 |
| 25 | 2 | | 40 | 86 | 50 | 5 | 9 |
| 25 | 5 | 6.88 | 40 | 95 | 51 | 6 | 9 |
| 25 | 10 | 9.18 | 40 | 97 | 50 | 6 | 9 |

### Example 9

Antioxidant Evaluation

A study was performed to identify antioxidant candidates for use in a high concentration poloxamer-188 LCMF formulation. The results of the oxygen content study of Example 8 were used to determine the level of oxygen needed to appropriately challenge the poloxamer solutions listed in Table 14. Antioxidants ascorbic acid, cysteine, citric acid, dextrose, dithiothreitol, histidine, malic acid, mannitol, methionine, sodium metabisulfate, and tartaric acid were added in different percentages. Formulation compositions can be found in Table 14. pH, dissolved oxygen, osmolality, and potency via ELSD were evaluated at t=0, 1 month, and 3 months stored at 40° C. The HPLC method parameters and gradient used are described in the Tables A & B below. Results can be found in Table C.

TABLE 14
Antioxidant Formulations For Evaluation

| LCMF | | | | |
|---|---|---|---|---|
| Poloxamer | Buffer | | | |
| 188 | species | | | Oxygen |
| Concentration | (20 mM, | | Antioxidant | content |
| (% w/v) | pH 6) | Antioxidant | Concentration | (mg/L) |
| 2 and 25 | Sodium | N/A | N/A | NMT 2 |
| | Phosphate | | | mg/mL |
| 2 and 25 | Sodium | N/A | N/A | 8-10 |
| | Phosphate | | | |
| 2 and 25 | Sodium | Alpha | 0.075% | 8-10 |
| | Phosphate | Tocopherol | | |
| 2 | Sodium | Ascorbic | 0.5% | 8-10 |
| | Phosphate | Acid | | |
| 2 | Sodium | BHA | 0.001% | 8-10 |
| | Phosphate | | | |
| 2 | N/A | Cysteine | 2% | 8-10 |
| 2 and 25 | Sodium | Cysteine | 2% | 8-10 |
| | Phosphate | | | |
| 2 and 25 | Sodium | Citric Acid | 10 mM | 8-10 |
| | Phosphate | | | |
| 2 | Sodium | Dextrose | 2% | 8-10 |
| | Phosphate | | | |
| 2 | Sodium | Dithiothreitol | 0.1 mM | 8-10 |
| | Phosphate | | | |
| 2 and 25 | Sodium | Histidine | 2% | 8-10 |
| | Phosphate | | | |
| 2 | Sodium | Malic Acid | 0.3% | 8-10 |
| | Phosphate | | | |
| 2 and 25 | Sodium | Mannitol | 1% | 8-10 |
| | Phosphate | | | |
| 2 | Sodium | Methionine | 0.2% | 8-10 |
| | Phosphate | | | |
| 2 | Sodium | Sodium | 0.02% | 8-10 |
| | Phosphate | Metabisulfate | | |
| 2 | Sodium | Tartaric | 6.0 g | 2% |
| | Phosphate | Acid | | |
| 15% MST- | 10 mM | Citric Acid | 0.576 g | 10 mM |
| 188 SCD | Citrate | | | |

TABLE A
HPLC Method Parameters

| Parameter | Condition |
|---|---|
| Column | Thermo-Scientific, Acclaim |
| | Surfactant, 5 μm, 4.6 × 250 mm |
| Mobile Phase A | 0.1% Acetic Acid in Water |
| Mobile Phase B | Isopropanol |
| Flow Rate | 0.6 mL/minute |
| Column Temperature | 40 ± 5° C. |
| Autosampler Temperature | Ambient |
| Detection | ELSD, N₂ 1.0 L/minute, Nebulizer |
| | Off, Evaporator 45° C. |
| Gain Setting | 20 |
| Run Time | 20 minutes |
| Injection Volume | 10 μL |
| Sample Diluent | D.I. Water |
| Needle Wash | Isopropanol |

TABLE B
HPLC Gradient Parameters

| Time | Flow | % Mobile | % Mobile |
|---|---|---|---|
| (Minutes) | (mL/min) | Phase A | Phase B |
| 0.0 | 0.60 | 90 | 10 |
| 1.0 | 0.60 | 90 | 10 |
| 7.0 | 0.60 | 30 | 70 |
| 8.5 | 0.60 | 5 | 95 |
| 9.0 | 0.60 | 5 | 95 |
| 9.1 | 0.60 | 90 | 10 |
| 20.0 | 0.60 | 90 | 10 |

TABLE C
Antioxidant Results

| | Time | | DO | % | Osmolality |
|---|---|---|---|---|---|
| Sample | Pull | pH | (mg/L) | Potency | (mmol/kg) |
| 2% MST-188 | t = 0 | 6.03 | 1.26 | 90.25 | 65 |
| Control | t = | 6.00 | 9.23 | 88.37 | 77 |
| | 1 mon. | | | | |
| 25% MST-188 | t = 0 | 6.01 | 1.71 | 96.74 | 548 |
| Control | t = | 5.99 | 9.30 | 85.94 | 528 |
| | 1 mon. | | | | |
| 2% MST-188 No | t = 0 | 6.04 | 8.78 | 88.84 | 66 |
| Antioxidant | t = | 4.98 | 9.30 | 82.84 | 76 |
| | 1 mon. | | | | |
| 25% MST-188 No | t = 0 | 6.04 | 9.23 | 98.39 | 537 |
| Antioxidant | t = | 4.31 | 9.06 | 87.46 | 550 |
| | 1 mon. | | | | |
| 2% MST-188 | t = 0 | 5.99 | 2.88 | 67.62 | 126 |
| Ascorbic Acid | t = | 5.66 | 9.06 | 71.00 | 134 |
| | 1 mon. | | | | |
| 2% MST-188 No | t = 0 | 6.02 | 6.50 | 94.65 | 169 |
| buffer, Cystine | t = | 5.78 | 9.06 | 97.83 | 159 |
| | 1 mon. | | | | |
| 2% MST-188 | t = 0 | 5.99 | 4.15 | 85.14 | 241 |
| Cystine | t = | 5.85 | 8.83 | 89.17 | 229 |
| | 1 mon. | | | | |
| 25% MST-188 | t = 0 | 6.04 | 3.90 | 92.94 | 873 |
| Cystine | t = | 5.73 | 8.83 | 86.38 | 823 |
| | 1 mon. | | | | |
| 2% MST-188 | t = 0 | 5.97 | 5.79 | 88.95 | 101 |
| Citric Acid | t = | 5.74 | 8.92 | 82.18 | 100 |
| | 1 mon. | | | | |
| 25% MST-188 | t = 0 | 5.95 | 8.56 | 84.89 | 588 |
| Citric Acid | t = | 5.64 | 8.82 | 85.40 | 615 |
| | 1 mon. | | | | |
| 2% MST-188 | t = 0 | 6.03 | 9.06 | 86.79 | 191 |
| Dextrose | t = | 5.84 | 8.78 | 88.53 | 183 |
| | 1 mon. | | | | |
| 2% MST-188 | t = 0 | 6.03 | 8.88 | 88.26 | 63 |
| Dithiothreitol | t = | 4.83 | 8.98 | 84.11 | 68 |
| | 1 mon. | | | | |
| 2% MST-188 | t = 0 | 6.04 | 9.18 | 81.59 | 242 |
| Histidine | t = | 5.95 | 8.87 | 82.03 | 237 |
| | 1 mon. | | | | |
| 25% MST-188 | t = 0 | 6.01 | 8.69 | 93.54 | 838 |
| Histidine | t = | 5.89 | 8.97 | 82.91 | 815 |
| | 1 mon. | | | | |
| 2% MST-188 | t = 0 | 6.02 | 8.96 | 85.94 | 118 |
| Malic Acid | t = | 5.73 | 8.93 | 83.05 | 140 |
| | 1 mon. | | | | |
| 2% MST-188 | t = 0 | 5.98 | 8.39 | 87.57 | 129 |
| Mannitol | t = | 5.66 | 9.27 | 86.38 | 131 |
| | 1 mon. | | | | |
| 25% MST-188 | t = 0 | 6.05 | 8.86 | 98.41 | 663 |
| Mannitol | t = | 4.55 | 9.24 | 93.16 | 637 |
| | 1 mon. | | | | |
| 2% MST-188 | t = 0 | 6.04 | 8.96 | 89.17 | 94 |
| Methionine | t = | 5.93 | 9.08 | 89.24 | 76 |
| | 1 mon. | | | | |
| 2% MST-188 | t = 0 | 5.99 | 1.00 | 82.27 | 74 |
| Sodium | t = | 3.54 | 9.16 | 80.28 | 72 |
| Metabisulfate | 1 mon. | | | | |
| 2% MST-188 | t = 0 | 5.99 | 8.80 | 75.10 | 363 |
| Tartaric Acid | t = | 5.79 | 9.11 | 73.18 | 358 |
| | 1 mon. | | | | |
| 15% MST-188 | t = 0 | 5.95 | 8.43 | 98.67 | 305 |
| Sodium | t = | 5.83 | 9.11 | 101.62 | 312 |
| Containing Drug, | 1 mon. | | | | |
| Citrate | | | | | |

### Example 10

pH Adjusting Agent

Meglumine was evaluated as a pH modifier to replace ammonium hydroxide because of the safety concerns and difficulty in handling concentrated ammonium hydroxide. Multiple sodium free poloxamer 188 drug product formulations were compounded under aseptic-like conditions and filled into 50-mL sterile vials for evaluation. Drug products with varying percentages of poloxamer 188, citric acid, and MgCl₂, were prepared and pH adjusted with ammonium hydroxide or meglumine. The MgCl₂ concentration was also lowered due to the results from the particulate formation study that indicated magnesium and citrate components of the formulation were likely the cause of the particle formation. These formulations were analyzed via blood smear testing as well as evaluated by an IV flow study. A second batch of sodium free poloxamer 188 formulations, pH adjusted with meglumine, were compounded under aseptic-like conditions and filled into 50-mL sterile vials. These formulations were also analyzed via blood smear testing.

Appearance, pH, dissolved oxygen, viscosity, density, osmolality, and potency via the ELSD method were evaluated for all formulations. Appearance, pH, and dissolved oxygen were measured in process. The formulation compositions and results can be found below in Table 15 and Table 16, respectively.

Meglumine is a suitable replacement for ammonium hydroxide. It is considerably easier to handle and adjustment of pH was more predictable. All poloxamer 188 containing formulations were compatible with whole blood.

TABLE 15
Formulation Compositions

| LCMF | Citric | | | |
|---|---|---|---|---|
| Poloxamer 188 | Acid | MgCl₂ | Meglumine | NH₄OH |
| 150 mg/mL | 30 mM | 1.40 mM | N/A | 676.2 mg |
| 200 mg/mL | 10 mM | 1.40 mM | N/A | 235.66 mg |
| 225 mg/mL | 10 mM | 1.40 mM | N/A | 244.90 mg |
| 250 mg/mL | 10 mM | 1.40 mM | N/A | 164.20 mg |
| 300 mg/mL | 10 mM | 1.40 mM | N/A | 239.0 mg |
| 200 mg/mL | 10 mM | N/A | N/A | 179.44 mg |
| 225 mg/mL | 10 mM | N/A | N/A | 254.86 mg |
| 250 mg/mL | 10 mM | N/A | N/A | 168.30 mg |
| 300 mg/mL | 10 mM | N/A | N/A | 197.80 mg |
| 150 mg/mL | 10 mM | 16.3 mM | 30 mM | N/A |
| 150 mg/mL | 30 mM | 1.40 mM | 73 mM | N/A |
| 150 mg/mL | 30 mM | N/A | 73 mM | N/A |
| 67 mg/mL | — | — | — | — |
| 200 mg/mL* | 10 mM | 3 mM | 24 mM | — |
| 225 mg/m*L | 10 mM | 3 mM | 24 mM | — |
| 250 mg/mL* | 10 mM | 3 mM | 24 mM | — |
| 250 mg/mL* | 30 mM | 30 mM | 86 mM | — |

¹Formulation was prepared diluting the original sodium containing formulation with ½ saline
*Samples were only evaluated by blood smear testing

TABLE 16
results of formulations from Table 15 evaluated by blood smear testing

| | | D.O. | Osmolality | % | Viscosity | Density |
|---|---|---|---|---|---|---|
| Appearance | pH | (mg/L) | (mMol/kg) | Potency | (cP) | (g/cm³) |
| Clear, | 6.07 | 1.75 | 252 | 94.1 | N/A | 1.0254 |
| colorless | | | | | | |
| solution | | | | | | |
| Clear, | 6.08 | 1.72 | 291 | 100.6 | 16.28 | 1.0308 |
| colorless | | | | | | |
| solution | | | | | | |
| Clear, | 5.86 | 1.45 | 364 | 103.5 | 21.74 | 1.0355 |
| colorless | | | | | | |
| solution | | | | | | |
| Clear, | 5.96 | 1.72 | 463 | 101.2 | 28.03 | 1.0396 |
| colorless | | | | | | |
| solution | | | | | | |
| Clear, | 5.97 | 1.70 | 729 | 69.4 | 46.67 | 1.0483 |
| colorless | | | | | | |
| solution | | | | | | |
| Clear, | 6.38 | 1.90 | 270 | 102.0 | 16.71 | 1.0310 |
| colorless | | | | | | |
| solution | | | | | | |
| Clear, | 6.21 | 1.06 | 347 | 101.9 | 21.51 | 1.0350 |
| colorless | | | | | | |
| solution | | | | | | |
| Clear, | 5.87 | 1.49 | 463 | 100.8 | 28.54 | 1.0396 |
| colorless | | | | | | |
| solution | | | | | | |
| Clear, | 6.39 | 1.86 | 708 | 123.1 | 45.76 | 1.0479 |
| colorless | | | | | | |
| solution | | | | | | |
| Clear, | 6.22 | 1.57 | 212 | 98.2 | 9.798 | 1.0255 |
| colorless | | | | | | |
| solution | | | | | | |
| Clear, | 6.04 | 1.85 | 271 | 99.0 | 10.33 | 1.0298 |
| colorless | | | | | | |
| solution | | | | | | |
| Clear, | 5.97 | 1.80 | 269 | 119.2 | 10.54 | 1.0299 |
| colorless | | | | | | |
| solution | | | | | | |
| Clear, | N/A | N/A | 154 | 122.4 | <10 | 1.0120 |
| colorless | | | | | | |
| solution | | | | | | |
| Clear, | 5.92 | 1.75 | 290 | 124.5 | 16.11 | 1.0319 |
| colorless | | | | | | |
| solution | | | | | | |
| Clear, | 5.85 | 1.72 | 392 | 187.2 | 20.66 | 1.0401 |
| colorless | | | | | | |
| solution | | | | | | |
| Clear, | 6.02 | 1.50 | 480 | 78.7 | 26.91 | 1.0468 |
| colorless | | | | | | |
| solution | | | | | | |
| Clear, | 5.73 | 1.68 | 762 | 118.3 | 27.24 | 1.0360 |
| colorless | | | | | | |
| solution | | | | | | |

### Example 11

Viscosity and Injectability

Viscosity and injectability were determined over a range of concentrations. The viscosity was measured with a rolling ball viscometer at 20° C. while the injectability was measured with a texture analyzer using a 27G1/2 needle and a 1 mL plastic syringe. Both properties increased exponentially when the concentration was increased linearly. Results are shown in Table 17.

TABLE 17
Injectability and viscosity of different concentrations of
Poloxamer-188

| LCMF Poloxamer 188 | Viscosity | |
|---|---|---|
| Concentration (wt/wt %) | (cP)¹ | Injectability (N)² |
| 2.5 | 1.51 | 2.21 |
| 5 | 2.30 | 2.80 |
| 10 | 4.83 | 3.67 |
| 15 | 9.07 | 5.26 |
| 20 | 16.49 | 7.75 |
| 25 | 27.45 | 13.22 |
| 30 | 44.91 | 23.58 |
| 35 | 74.60 | 33.51 |
| 40 | 169.60 | >65 |

¹Centipose
²Newtons

To further evaluate the impact increased viscosity may have on the rate of delivery an additional study was conducted. Filtered and unfiltered IV administration sets were used to deliver a variety of MST-188 (LCMF poloxamer-188) formulations at a target rate of 10 mL/min (600 mL/hr) (Tables 18 and 19). While none of the formulations achieved the target delivery rate, there was no observable correlation between concentration of MST-188 and the rate of delivery.

Filtered Line, Rate: 10 mL/min

TABLE 18
delivery rates of LCMF poloxamer 188 formulations-filtered line

| | | Volume | Rate |
|---|---|---|---|
| | Time | Collected | (mL/ |
| Formulation | (mM) | (mL) | min) |
| SWFI | 1 | 9 | 9 |
| 150 mg/mL LCMF Poloxamer 188, 30 | 1 | 9 | 9 |
| mMol Citric Acid, 1.4 mMol MgCl₂ | | | |
| 200 mg/mL LCMF Poloxamer 188, 10 | 1 | 7.5 | 7.5 |
| mMol Citric Acid, 1.4 mMol MgCl₂ | | | |
| 225 mg/mL LCMF Poloxamer 188, 10 | 1 | 9 | 9 |
| mMol Citric Acid, 1.4 mMol MgCl₂ | | | |
| 250 mg/mL LCMF Poloxamer 188, 10 | 1 | 8 | 8 |
| mMol Citric Acid, 1.4 mMol MgCl₂ | | | |
| 300 mg/mL LCMF Poloxamer 188, 10 | 1 | 8 | 8 |
| mMol Citric Acid, 1.4 mMol MgCl₂ | | | |
| 200 mg/mL LCMF Poloxamer 188, 10 mM | 1 | 8.5 | 8.5 |
| Citric Acid | | | |
| 225 mg/mL LCMF Poloxamer 188, 10 mM | 1 | 9 | 9 |
| Citric Acid | | | |
| 250 mg/mL LCMF Poloxamer 188, 10 mM | 1 | 8 | 8 |
| Citric Acid | | | |
| 300 mg/mL LCMF Poloxamer 188, 10 mM | 1 | 8.5 | 8.5 |
| Citric Acid | | | |
| 150 mg/mL LCMF Poloxamer 188, 10 | 1 | 8.5 | 8.5 |
| mMol Citric Acid, 16.3 mMol MgCl₂, 30 | | | |
| mMol Meglumine | | | |
| 150 mg/mL LCMF Poloxamer 188, 30 | 1 | 8 | 8 |
| mMol Citric Acid, 1.4 mMol MgCl₂, 73 | | | |
| mMol Meglumine | | | |
| 150 mg/mL LCMF Poloxamer 188, 30 | 1 | 8.5 | 8.5 |
| mMol Citric Acid, 73 mMol Meglumine | | | |
| 67 mg/mL Original Formulation¹ | 1 | 9 | 9 |

¹Formulation was prepared diluting the original sodium containing formulation with ½ saline

TABLE 19
delivery rates of LCMF poloxamer 188 formulations
unfiltered line, Rate: 10 mL/min

| | | Volume | Rate |
|---|---|---|---|
| | Time | Collected | (mL/ |
| Formulation | (min) | (mL) | min) |
| SWFI | 1 | 8 | 8 |
| 150 mg/mL, 30 mMol | | | |
| Citric Acid, 1.4 mMol | 1 | 8 | 8 |
| MgCl₂ | | | |
| 200 mg/mL, 10 mMol | | | |
| Citric Acid, 1.4 mMol | 1 | 8 | 8 |
| MgCl₂ | | | |
| 225 mg/mL, 10 mMol | | | |
| Citric Acid, 1.4 mMol | 1 | 8 | 8 |
| MgCl₂ | | | |
| 250 mg/mL, 10 mMol | | | |
| Citric Acid, 1.4 mMol | 1 | 8 | 8 |
| MgCl₂ | | | |
| 10 mMol Citric Acid, 1.4 | | | |
| mMol MgCl₂ | 1 | 8.5 | 8.5 |
| 200 mg/mL, 10 mM Citric | 1 | 9 | 9 |
| Acid | | | |
| 225 mg/mL, 10 mM Citric | 1 | 8.5 | 8.5 |
| Acid | | | |
| 250 mg/mL, 10 mM Citric | 1 | 8 | 8 |
| Acid | | | |
| 300 mg/mL, 10 mM Citric | 1 | 9 | 9 |
| Acid | | | |
| 150 mg/mL, 10 mMol | 1 | 9 | 9 |
| Citric Acid, 16.3 mMol | | | |
| MgCl₂, 30 mMol | | | |
| Meglumine | | | |
| 150 mg/mL, 30 mMol | 1 | 8.5 | 8.5 |
| Citric Acid, 1.4 mMol | | | |
| MgCl₂,73 mMol | | | |
| Meglumine | | | |
| 150 mg/mL, 30 mMol | 1 | 8.5 | 8.5 |
| Citric Acid, 73 mMol | | | |
| Meglumine | | | |
| 67 mg/mL Original | 1 | 8.5 | 8.5 |
| Formulation¹ | | | |

¹Formulation was prepared diluting the original sodium containing formulation with ½ saline

### Example 12

Continuous Process Purification of Poloxamer 188 by Extraction with Methanol/Supercritical CO₂ Co-Solvent

A continuous process purification of poloxamer 188 by extraction with a methanol/supercritical CO₂ co-solvent was evaluated. The continuous process allows for high throughput. A feed solution of poloxamer 188 (Asahi Denka Kogyo, Japan) in methanol was pumped at the midpoint of a high pressure extraction column packed with suitable packing material. Supercritical CO₂ (Carboxyque, France) mixed with methanol was pumped through the extraction column from the bottom in a counter current fashion (flow rate=30 kg/h to 40 kg/h). The average concentration of methanol was 13%, and was provided as a gradient of 9 to 13.2 weight %. The gradient was controlled by controlling the methanol, CO₂ and poloxamer flow rates at the feed port in the middle of the column and the CO₂/methanol flow rate introduced at the bottom of the column. The column pressure was 200±15 bars. The temperature of the feed solution and supercritical CO₂/methanol solvent was a gradient of 36 to 44° C. The column jacket temperature and extraction temperature were a gradient of 36 to 54° C.

Low molecular weight (LMW) polymers were removed at the top of the column while purified product containing methanol was removed from the bottom of the extraction column. The purified product was collected hourly and precipitated under reduced pressure via a Particle from Gas Saturated Solutions (PGSS) technique. The purified product was dried under vacuum at not more than 40° C. to remove residual methanol.

The approximate yield of purified poloxamer per feed was approximately 60%. The peak average molecular weight was approximately 9,000 Daltons. Low molecular weight components (less than 4,500 Daltons) were approximately 1.0%. Polydispersity was approximately 1.0.

### Example 13

12-L Scale Dual-Step Extraction Batch Process Purification of Poloxamer 188

A 12-L extraction system containing a stirred extraction vessel, cyclone separators, CO₂ solvent circulation and methanol co-solvent system is tested for leaks. The extraction system is pressurized with CO₂ to 310±15 bars at the start of the campaign. Methanol (2 kg) is dispensed into the feed mix tank with liner and warmed to 40° C. Approximately 3700 grams of poloxamer 188 is added to the feed tank and stirred until completely mixed. 5100 grams of the mixed solution is pumped into the extractor. The CO₂ flow rate is maintained at 390 gm/min. Two (2) successive extractions are performed by adjusting the methanol concentration. Extraction is conducted for 12 hours±30 minutes at 7.6% MeOH/CO₂ with a methanol flow rate of 27.6±1.0 gm/min. Extraction is continued for 12 hours±15 minutes at 8.6% MeOH/CO₂ at a methanol flow rate of 36.6±1.0 gm/min.

After the 24-hr purification, the extractor is discharged through the rapid depressurization system (Particle from Gas Saturated Solutions (PGSS)) and the wet product is collected in the liners. A sample of wet product (˜600 gm) is transferred to a flask and dried using a rotary evaporator for approximately 3 hours at room temperature and moderate vacuum, followed by 30 minutes at room temperature and high vacuum and 30 additional minutes at 35° C. The dried product is collected and tested by Gel Permeation Chromatography (GPC) for molecular weight distribution. No low molecular weight (LMW) components are detected in the purified product. The purified product contained approximately 4.5% high molecular weight (HMW) components. Methods for GPC are known in the art, e.g. Grindel et al. (2002) (Biopharmaceutics & Drug Disposition, 23:87-103).

### Example 14

Batch Process Purification of Poloxamer 188 by Extraction with Methanol/Supercritical CO₂ Co-Solvent

A batch process purification of poloxamer 188 by extraction with a methanol/supercritical CO₂ cosolvent was evaluated. Poloxamer 188 (Asahi Denka Kogyo, Japan) was purified by adjusting the solvent characteristics by controlling the extraction solvent temperature, pressure and methanol co-solvent content. The processes differed in the pressure and the co-solvent content.

Poloxamer 188 (13-14 kg) was mixed with methanol solvent in a high pressure extraction vessel. A co-solvent of methanol and supercritical CO₂ (BOC gases, USA) was mixed and pumped through the extraction vessel. The extraction was started with a lower methanol concentration that was successively increased while monitoring the composition of the fraction removed during the extraction. The average methanol concentration was 7.3% (by weight). The concentration was increased stepwise from 6.6% to 7.6% and to 8.6%. The extraction vessel pressure was 300±15 bars. The methanol/supercritical CO₂ solvent temperature and extractor jacket temperature were 40±5° C. The extraction temperature was adjusted to 35-45° C. The eluted fractions were analyzed by Gel Permeation Chromatography (GPC). The molecular weight distribution of the purified poloxamer 188 recovered from the extraction vessel was narrower than for the starting material.

The resulting yield was approximately 75%. The peak average molecular weight was approximately 9,000 Daltons. Low molecular weight components (less than 4,500 Daltons) were approximately 1.0%. Polydispersity was approximately 1.0.

### Example 15

12-L Scale Multi-Step Extraction Batch Process Purification of Poloxamer 188 and Analysis by Gel Permeation Chromatography (GPC)

Four batches of poloxamer 188 were purified by SFE Batch Process in a 12 liter extraction vessel. Each batch was purified as described below. The system was pressurized with CO₂ and the pressure was maintained above 900 psig (63 bars) between batches. Methanol (2000±20 gm) was dispensed into the feed mix tank with liner and warmed to 40° C. Poloxamer 188 (3696±20 gm) was dispensed into the feed tank and stirred until mixed. Ninety percent (90%) of the poloxamer 188 solution was pumped into the extractor, and the system was pressurized to 310±15 bars. The CO₂ flow rate was maintained at 390 gm/min. Three (3) successive extractions were performed by adjusting the methanol concentration with a controlled stepwise increase through 6.6 weight %, 7.6 weight % or 8.6 weight %. At each methanol concentration, extraction was conducted for a defined time period as described below (Table 19). In-process samples were collected from the bottom of the extractor after the designated times during each extraction.

TABLE 20
Extraction conditions for multi-step purification of poloxamer 188

| | | In-process | | | |
|---|---|---|---|---|---|
| | | sample | CO₂ flow | Methanol | Percent |
| | Time | collection | rate | flow rate | methanol |
| Extraction | (hours) | times (hr) | (gm/min) | (gm/min) | in CO₂ |
| 1 | 12 (±0.5) | 4, 8 and 12 | 390 | 27.6 | 6.6% |
| | | | | (±1.0) | |
| 2 | 10 (±0.5) | 3, 6 and 10 | 390 | 32.1 | 7.6% |
| | | | | (±1.0) | |
| 3 | 4 (±0.25) | 2 and 4 | 390 | 36.6 | 8.6% |
| | | | | (±1.0) | |

At the end of the 26-hour purification process, the extractor was discharged through the rapid depressurization system and the wet product was collected in the liners. A sub-lot of wet product (˜600 g) was transferred to a flask and dried using a rotary evaporator for approximately 3 hours at room temperature and moderate vacuum, followed by 30 minutes at room temperature and high vacuum and an additional 30 minutes at 35° C. and high vacuum. The dried product was collected as a sub-lot. This drying process was repeated with the remaining wet product to make 3 sub-lots of dried product. The 3 sub-lots were combined in a 10 L drum and mixed for 30 minutes to produce purified poloxamer 188. The yield per feed was approximately 55%.

The starting and purified poloxamer 188 products were assessed by Gel Permeation Chromatography (GPC). The GPC trace of the starting poloxamer 188 shows a narrow molecular weight distribution with a small additional peak at the low molecular weight side. The area under the curve for the low molecular weight component is approximately 4-7%, with an average molecular weight of less than 4,500 Daltons. In comparison, the GPC trace of the purified poloxamer 188 shows a narrow molecular weight distribution with significantly smaller amounts of low molecular weight peak (less than 1.5% of the area of the main peak).

### Example 16

Preparation and Administration of Long Circulating Material Free (LCMF) Poloxamer 188

A multi-step extraction batch process of poloxamer 188 was performed with extraction conducted at a pressure of 247±15 atm (approximately 200-260 bar) and a controlled step-wise increase of methanol of 7.4, 9.1 and 10.7 weight % methanol. Before purification, the poloxamer 188 raw material (BASF Corporation, Washington, N.J.) was characterized by Gel Permeation Chromatography (GPC). Molecular weight analysis demonstrated that raw material had an average molecular weight of the main peak of about 8,500±750 Da, no more than 6.0% low molecular weight (LMW) species of less than 4,500 Da and no more than 1% high molecular weight species (HMW) greater than 13,000 Da. In addition, the polydispersity was no more than 1.2.

A 50-L, high pressure, stainless steel, extractor vessel was charged with 14 kg of commercial grade poloxamer 188 (BASF Corporation, Washington, N.J.) and 7 kg of methanol, pressurized with CO₂ (49±10 atm, i.e. 720±147 psi) (Messer France, S.A.S., Lavera, France) and heated to 35° C. to 50° C. for 40-80 minutes until a homogenous solution was obtained. CO₂ (supplied either from a main supply tank or via recycling through an extraction system), was cooled in a heat exchanger and fed into a temperature-controlled, high pressure, stainless steel, solvent reservoir. A high-pressure pump increased the pressure of liquid CO₂ to the desired extraction pressure. The high pressure CO₂ stream was heated to the process temperature by a second heat exchanger. Methanol (Merck KGaA, Darmstadt, Germany) was fed from a main supply tank into the CO₂ solvent stream to produce the extraction methanol/CO₂ co-solvent, which was fed through inlet systems into the extractor vessel as a fine mist at a pressure of 247±15 atm (3600±psi) or 240 to 260 bar and a temperature of 40° C.

A 7.4% methanol/CO₂ extraction cosolvent was percolated through the poloxamer solution for 3 hours at a methanol flow rate typically at 8 kg/hr (range 6.8 kg/hr to 9.2 kg/hr; 108 kg/hr total flow rate). The extraction continued with a 9.1% methanol/CO₂ cosolvent for 4 more hours at a methanol flow rate typically at 10 kg/hour (range of 8.5 kg/hr to 11.5 kg/hr; 110 kg/hr total flow rate). The extraction further continued with a 10.7% methanol/CO₂ co-solvent for 8 more hours at a methanol flow rate typically at 12 kg per hour (range of 10.2 kg/hr to 13.8 kg/hr; 112 kg/hr total flow rate). Throughout the extraction process, extraction of soluble species were continuously extracted from the top of the extractor. The extraction solvent was removed from the top of the extractor and passed through two high pressure, stainless steel, cyclone separators arranged in series to reduce system pressure from 247 atm (3600 psi) to 59 atm (870 psi) and then from 59 atm to 49 atm (720 psi) and to separate CO₂ from the methanolic stream. The separated CO₂ was condensed, passed through the heat exchanger and stored in the solvent reservoir. Pressure of the methanol waste stream was further reduced by passing through another cyclone separator. The purified poloxamer 188 remained in the extractor.

After extraction, the purified poloxamer 188 solution was discharged from the bottom of the extractor into a mixer/dryer unit equipped with a stirrer. The poloxamer 188 product was precipitated under reduced pressure via a Particle from Gas Saturated Solutions (PGSS) technique. The precipitate contained approximately 20% to 35% methanol. The purified poloxamer 188 was dried under vacuum at not more than 40 or 45° C. to remove residual methanol. The feed yield of the product gave an average yield of 65%.

Molecular weight analysis of the purified product as determined by GPC demonstrated that the purified product met the acceptance specifications. There was an average molecular weight of the main peak of about 8,500±750 Da and an average molecular weight average of 8,500±750 Da, no more than 1.5% low molecular weight (LMW) species of less than 4,500 Da and no more than 1.5% high molecular weight species (HMW) greater than 13,000 Da. In addition, the polydispersity was no more than 1.05. Thus, the results showed that the procedures resulted in a measurable reduction in the LMW species, and an improvement in the polydispersity of the purified product.

The resulting purified poloxamer 188 was formulated into a clear, colorless, sterile, non-pyrogenic, aqueous solution containing the purified poloxamer at 150 mg/ml, sodium chloride at 3.08 mg/ml, sodium citrate (dihydrate) at 2.38 mg/ml, citric acid anhydrous at 0.366 mg/ml in water for injection. The solution was sterile filtered and filled into 100 ml glass vials, covered with a nitrogen blanket, and closed with a butyl rubber stopper and aluminum overseal. The resulting osmolarity of the solution was approximately 312 mOsm/L. The LCMF poloxamer-188 composition did not contain any bacteriostatic agents or preservatives.

Purified LCMF poloxamer 188 generated as described above was administered intravenously to 62 healthy volunteers as part of assessment to determine its effect on the QT/QTc interval. Eight of the 62 subjects were randomly selected for quantitative analysis of the plasma poloxamer levels using an HPLC-GPC method. Following administration blood samples were obtained by venipuncture into heparin anticoagulated tubes at baseline, during drug administration (hours 1, 2, 3, 4, 5, and 6) and post administration at hours 1, 1.5, 2, 2.5, 5, 6, and 18. Plasma was separated by centrifugation and stored frozen until analysis. The purified poloxamer 188 was administered as either a high dose of a loading dose of 300 mg/kg/hr for one hour followed by a maintenance dose of 200 mg/kg/hr for 5 hours or a lower dose of 100 mg/kg for 1 hour followed by 30 mg/kg/hr for 5 hours. A mean maximum concentration (Cmax) of the administered purified poloxamer 188 of 0.9 mg/mL was attained by the end of the one hour loading infusion. The mean concentration at steady state (Css) was about 0.4 mg/ml was attained during maintenance infusion. The plasma concentration declined rapidly following discontinuation of the maintenance infusion. The LCMF product purified as described above did not demonstrate the longer circulating higher molecular weight material, observed with prior poloxamer 188 and as defined herein, in the plasma.

To confirm the absence of such longer circulating material in plasma, plasma from subjects receiving the higher dose were similarly studied using HPLC-GPC. The chromatograms show that over time the high molecular weight portion of the poloxamer 188 polymeric distribution declines in relative proportion to the main peak and lower molecular weight components. Thus, the polymeric distribution shows clears from the circulation in a substantially uniform manner The results also show that the higher molecular weight species do not exhibit a longer circulating half-life (relative to the other polymeric components) and do not accumulate in the circulation following intravenous administration.

The (LCM-containing) purified poloxamer 188 was administered to 6 healthy volunteers as an intravenous loading dose of 100 mg/kg/hr for one hour followed by 30 mg/kg/hr for 48 hours as part of a safety and pharmacokinetics study (Grindel et al. (2002) Biopharmaceutics & Drug Disposition, 23:87-103). Blood samples were obtained by venipuncture into EDTA anticoagulated tubes prior to drug administration (baseline), during administration (at 1 hour, 6 hours, 12 hours 18 hour 24 hours 36 and 48 hours) and at 30 minutes, 1 hour, 1.5 hours, 2 hours, 4 hours, 6 hours, 8 hours, 12 hours, 14 hours, 20 hours and 24 hours post drug administration. Plasma was separated and stored frozen until analysis using an HPLC-GPC method. Analysis of the plasma samples revealed the clearance kinetics of the main peak and the HMW peak for the (LCM-containing) purified poloxamer 188

Following administration at the above dose, the HMW component (detected in the HPLC-GPC assay as a peak of approximately 16,000 daltons) was accumulating during the drug administration period and did not reach its mean Cmax concentration of 225 μg/ml (n=6) until 2 hours after the end of drug administration. By 6 hours after discontinuation of infusion, mean plasma levels remained at 202 μg/ml, a concentration that had declined by only about 10% from the Cmax value. Over the 24 hour post infusion blood collection period, mean plasma levels only declined by 22.5% to a plasma concentration of 165 μg/ml. Based on these changes in the plasma concentration time course the elimination half-life of >48 hours is estimated.

Following administration at the dose above, the main peak achieved an apparent mean steady state concentration of 522 μg/ml (n=6) that was maintained during drug infusion. One hour after discontinuation of infusion, plasma levels dropped from the steady state concentration by 52% to 255 μg/ml. By 6 hours after discontinuation, plasma levels had dropped by 85% to 81 μg/ml. By 24 hours post infusion, plasma levels declined by 96% to a plasma concentration of about 19 μg/ml (n=6). Based on these changes in the plasma concentration time course the half-life is estimated to be about 5 hours

LCMF poloxamer, was administered to 62 healthy volunteers at a dose of 300 mg/kg for one hour followed by 200 mg/kg/hr for 5 hours as part of assessment to determine its effect on the QT/QTc interval as previously described. Eight of the 62 subjects were randomly selected for quantitative analysis of the plasma poloxamer levels using a similar HPLC-GPC method as above but with improved linearity at lower plasma levels.

Following administration at the above dose, the HMW component, which was detected in the HPLC-GPC assay as a peak of approximately 16,000 daltons, accumulated to a small extent during drug administration, and achieved its Cmax (mean value of 117 μg/ml, n=8) by end infusion. By 1 hour after discontinuation of drug administration, plasma levels had declined by 27% from the Cmax value to 86 μg/ml. By 6 hours after the end of drug administration, mean plasma levels had declined by 71% from the Cmax value to 34 μg/ml. By 18 hours after the end of infusion, the mean plasma level had declined by 82% to a concentration of 19 μg/ml (n=8). Based on these changes in the plasma concentration over time, the elimination half-life for the HMW component was estimated to be between 6-9 hours.

Following administration at the dose above, the main peak achieved an apparent mean steady state concentration of 2,637 μg/ml that was maintained during the 6 hour infusion period (n=8). One hour after discontinuation of infusion, mean plasma levels had decreased from steady state by 67% to 872 μg/ml and by 6 hours after discontinuation, mean plasma levels had declined by 93% (from steady state) to 184 μg/ml. By 18 hours after discontinuation of infusion, mean plasma levels declined by over 98% (from steady state) to a plasma concentration of about 34 μg/ml (n=6). Based on these changes in the plasma concentration time course, the elimination half-life is estimated to be about 3 hours

A comparison of the relative rates of clearance from the plasma at similar time points following administration is shown below. The data demonstrate a marked difference in the rate of decline in plasma concentration between (LCM-containing) purified poloxamer 188 and the LCMF poloxamer 188, demonstrating that LCMF poloxamer 188 clears faster. The difference is apparent for the HMW peak and for the main peak. The difference is most apparent for the HMW peak (Table 21).

TABLE 21
Clearance rates from plasma of LCMF and LCM poloxamer 188

| | HMW Peak | | Main Peak | |
|---|---|---|---|---|
| | | (LCM- | | (LCM- |
| | | containing) | | containing) |
| | | purified | | purified |
| | LCMF | poloxamer 188 | LCMF | poloxamer 188 |
| % decrease 1 hr | 27 | 0 | 67 | 52 |
| | 71 | 10 | 93 | 85 |
| Apparent | 6-9 hours | >48 hours | About 3 | About 5 hours |
| elimination t1/2 | | | hours | |

In reversed phase chromatography there is a hydrophobic stationary phase (the column) and a more polar mobile phase. Because of this “reversed” phase condition, RP-HPLC is commonly used to separate compounds based on relative hydrophobicity. More hydrophobic compounds exhibit a longer column retention time compared to more hydrophilic compounds.

The following HPLC conditions were used to compare to compare column retention times for various poloxamers with known differences in their hydrophilic/lipophilic balance (HLB), along with purified poloxamer 188 containing LCM and the LCMF poloxamer 188 (Table 22).

TABLE 22
HPLC conditions for comparing column retention
times for various poloxamers

| Column | Xterra RP18, 3.5 um, 4.6 × 100 mm | |
|---|---|---|
| Mobile Phase | A: 0.1% HOAc in Water | |
| | B: Acetonitrile | |
| Gradient | Time | % B |
| | 0 | 50 |
| | 1.0 | 50 |
| | 15.0 | 90 |
| | 16.0 | 90 |
| | 16.1 | 50 |
| | 20.0 | 50 |

| | |
|---|---|
| Flow Rate | 0.50 ml/min |
| Column Temp | 40° C. |
| ELS*Detection | N₂: 0.5 liter/minute, Nebulizer: 75° C., |
| | Evaporator: 75° C. |
| Sample Preparation | Drug Product—No dilution |
| | Purified Poloxamer 188, 150 mg/mL |
| | in 10 mM Sodium Citrate |
| | pH 6 |
| Injection Volume | 10 μL |

*ELS = evaporative light scattering

The results show that the LCMF poloxamer 188 is different from prior art purified poloxamer 188. It has different pharmacokinetic properties, which reflect that it is more hydrophilic than the prior art material that contains the longer circulating material.

RP-HPLC chromatograms for a highly hydrophilic polymer (PEG 8000), the LCMF poloxamer 188, the LCM-containing purified poloxamer 188, and two poloxamers with decreasing HLB values (increasing hydrophobicity), Poloxamer 338 and Poloxamer 407, respectively, were obtained. The most hydrophilic polymer, PEG 8000, exhibits little retention on the column consistent with its highly hydrophilic nature. Poloxamer 338 (HLB>24) and Poloxamer 407 (HLB 18-23) exhibit far longer retention times (add the t_{R} and k′ values) in accord with their known HLB values. The LCMF purified poloxamer 188 elutes more quickly than the LCM-containing purified poloxamer 188, (the average t_{R} and k′ for LCMF purified poloxamer is about 8.8 (8.807) and about 3.2 (3.202), respectively, compared to about 10.0 (9.883) and 3.7 (3.697) for LCM containing purified poloxamer) indicating that the LCMF poloxamer 188 is relatively more hydrophilic than the LCM containing purified poloxamer 188.

Chromatograms for 3 different lots of purified LCMF poloxamer 188 and 2 different lots of purified (LCM-containing) poloxamer 188 were obtained. These results demonstrate a robust reproducibility for the different lots of materials, and shows that the difference between the two materials cannot be accounted for by assay variability. These results demonstrate that the polymeric distribution of LCMF poloxamer 188 is more hydrophilic than purified poloxamer 188.

As described herein, the LCMF poloxamer 188 exhibits a markedly different pharmacokinetic behavior following administration to human subjects when compared to purified poloxamer 188, which contains the longer circulating material (LCM) following in vivo administration. The data provided in this example indicate that LCMF poloxamer 188 is more hydrophilic compared to purified poloxamer 188 that gives rise to the longer circulating material.

The polymeric size distribution of purified variants of poloxamer 188 (purified LCM-containing poloxamer 188, and the LCMF poloxamer 188) is similar with regard to size as shown by HPLC-GPC (Table 23). Both meet the criteria:

TABLE 23
polymeric size distribution of purified poloxamer variants

| | Acceptance | |
|---|---|---|
| Test Attribute | Criteria | Test Method |
| Molecular Weight | | |
| Analysis | | |
| Peak MW | 8500 ± 750 Da | HPLC-GPC |
| Weight Average | 8500 ± 750 Da | |
| MW | NMT* 1.5% | |
| % LMW (<4500 Da) | NMT 1.5% | |
| % HMW (>13000 | NMT 1.05 | |
| Da) | | |
| Polydispersity | | |

*NMT = Not More Than

While the polymeric size distribution, as shown by HPLC-GPC, of both purified poloxamers is similar, as demonstrated by the RP-HPLC herein, the molecules that comprise the polymeric distribution of LCMF poloxamer 188 are more hydrophilic.

When injected into an animal a more hydrophilic polymeric distribution clears from the circulation at a faster rate. This accounts for the decreased presence of a longer circulating material in the LCMF poloxamer 188 preparation. The results also indicate that, as observed and described above, the main peak of the polymeric distribution clears faster. For example, the plasma concentration time course data from a clinical trial show a shorter elimination half-life for the main peak and the high molecular weight peak of the LCMF poloxamer 188 compared to the purified poloxamer 188 containing LCM.

Since the rheologic, cytoprotective, anti-adhesive and antithrombotic effects of P188 are optimal within the predominant or main copolymers of the distribution, which are approximately 8,400 to 9400 Daltons (which have a circulating half-life of about 4-7 hours), the presence of larger, more hydrophobic, longer circulating half-life components of poloxamer 188 is not desirable. For example, among the desired activities of P188 is its rheologic effect to reduce blood viscosity and inhibit red blood cell (RBC) aggregation, which account for its ability to improve blood flow in damaged tissues. In contrast, more hydrophobic, higher molecular weight poloxamers such as P338 (also called Pluronic® F108) and P308 (Pluronic® F98), increase blood viscosity and RBC aggregation (Armstrong et al. (2001) Biorheology, 38:239-247). This is the opposite effect of P188 and indicates that higher molecular weight, hydrophobic poloxamer species may have undesirable biological effects.

### Example 17

Batch Process Purification of Poloxamer 188 by Extraction with Methanol/High Pressure CO₂ Co-Solvent

A batch process purification of poloxamer 188 by extraction with a methanol/high pressure CO₂ co-solvent is evaluated. Poloxamer 188 (13-14 kg) is purified by extraction with a methanol/high pressure CO₂ solvent. Poloxamer 188 is stirred with methanol in a high pressure extraction vessel until mixed. A co-solvent of methanol and high pressure CO₂ is pumped through the extraction vessel. The solvent characteristics of the extraction solvent are adjusted by controlling the extraction solvent temperature, pressure and the amount of methanol co-solvent. Specifically, the combination of these three parameters are selected for removal of low molecular weight (LMW) and high molecular weight (HMW) components from the commercial-grade poloxamer 188. The starting concentration of methanol is approximately 2.5 wt % and is successively increased in increments up to 25 wt %. The Extraction vessel pressure is 75±10 bars, and the extraction temperature, methanol/CO₂ co-solvent temperature and extractor jacket temperature is 20-25° C. The extraction process is done in a sequential fashion to successively remove various components from the extractor.

The Extraction solvent is removed and eluted fractions were analyzed by Gel Permeation Chromatography (GPC). After purification, the purified poloxamer 188 is recovered from the extraction vessel and analyzed by GPC. Initially, low molecular weight (LMW) components are removed during extraction and the main fraction is removed at higher concentrations of methanol. High molecular weight components are removed at the later stages of the extraction process. The molecular weight distribution of the purified poloxamer 188 is narrower than for the starting material.

The yield of the polymer is estimated to be 60 to 80% with less than 1.5% low molecular weight components (less than 4,500 Daltons). The peak average molecular weight is about 8,500±750 Daltons.

### Example 18

Effects of Acute Intravenous Infusion of a 15% Sodium Free LCMF Poloxamer-188 Formulation on Left Ventricular Function in Dogs with Advanced Heart Failure.

Methods

The study was performed in 21 dogs with advanced heart failure (HF) produced by multiple sequential intracoronary microembolizations (LV ejection fraction ≤30%) (1). Dogs were randomized into 3 groups. Group-I (n=7) were treated with intravenous infusions of a 15% sodium free LCMF poloxamer-188 formulation (as described in Example 2) (450 mg/kg) administered over a period of 2 hours with complete hemodynamic follow-up at 24 hours, 1 week and 2 weeks post infusion. Group-II (n=7) were treated with intravenous infusions of MST-188 at a lower dose of the 15% sodium free LCMF poloxamer-188 formulation (225 mg/kg) also administered over a period of 2 hours with complete hemodynamic follow-up at 24 hours, 1 week and 2 weeks post infusion. Group-III (n=7) were treated with an intravenous infusion of normal saline also administered for 2 hours with complete hemodynamic follow-up at 24 hours, 1 week and 2 weeks. The volume of saline administered over the course of 2 hours was identical to drug volume administered over 2 hours in Groups I and II. Group-III served as a placebo-control. Hemodynamic, ventriculographic, echocardiographic, and electrocardiographic measurements were made at baseline, prior to drug administration and were repeated at the end of 2 hours of drug infusion. Peripheral venous blood samples were obtained at baseline and at the end of 2 hours of drug infusion and at 24 hours, 1 week and 2 weeks after drug infusion. Blood samples (at least 10 mL) were centrifuged at 3000 rpm for 10 minutes and plasma withdrawn and placed in cryo-storage tubes and stored upright at −70° C. until needed. Plasma samples were used to assess troponin-I (TnI) and n-terminal pro brain natriuretic peptide (nt-pro BNP).

All hemodynamic, ventriculographic, echocardiographic, electrocardiographic and plasma biomarker measurements were repeated in all study dogs at 24 hours, 48 hours, 1 week and 2 weeks after completion of the 2 hours of drug infusion. All measurements were performed under general anesthesia and sterile conditions. Induction of anesthesia was initiated with intravenous hydromorphone (0.22 mg/kg) and diazepam (0.17 mg/kg) and plane of anesthesia was maintained with 1-1.5% isofluorane. Studies were initiated once the protocol was approved by Henry Ford Health System Institutional Animal Care and Use Committee. All studies conformed to the National Institute of Health “Guide and Care for Use of Laboratory Animals” (NIH publication No. 85-23).

Hemodynamic and Ventriculographic Measurements

All hemodynamic measurements were made during left and right heart catheterizations in anesthetized dogs at each specified study time point. The following parameters will be evaluated in all dogs: 1) aortic and LV pressures using catheter tip micromanometers (Millar Instruments), 2) peak rate of change of LV pressure during isovolumic contraction (peak +dP/dt) and relaxation (peak dP/dt), 3) LV end diastolic pressure, and 4) cardiac output (CO). In addition, the following parameters were calculated: 1) LV stroke volume (SV) and 2) systemic vascular resistance (SVR).

Left ventriculograms were performed during cardiac catheterization after completion of the hemodynamic measurements. Ventriculograms were performed with the dog placed on its right side and were recorded on digital media at 30 frames/sec during a power injection of 20 ml of contrast material (ISOVUE-300, Bracco Diagnostics, Inc., Princeton, N.J.). Correction for image magnification was made using radiopaque markers placed on the distal end of the LV ventriculographic catheter. LV end systolic (ESV) and end diastolic (EDV) volumes were calculated from angiographic silhouettes using the area length method (2). Premature beats and postextrasystolic beats were excluded from the analysis. LV ejection fraction (EF) was calculated as the ratio of the difference of end diastolic and end systolic volumes to end diastolic volume times 100.

Echocardiographic and Doppler Measurements

Echocardiographic and Doppler studies were performed in all dogs at all specified study time points using a VIVID 7 ultrasound system (General Electric) with a 3.5 MHZ transducer. All echocardiographic measurements were made with the dog placed on its right side and recorded on a digital media for subsequent off line analysis. LV fractional area of shortening (FAS), a measure of LV systolic function, was measured from a short axis view at the level of the papillary muscles. LV major and minor semiaxes were measured and used for calculation of LV end-diastolic circumferential wall stress (EDWS) (3). Wall stress will be calculates as follows: Stress=Pb/h(1−h/2b)(1−hb/2a2), where P is LV end-diastolic pressure, a is LV major semiaxis, b is LV minor semiaxis, and h is LV wall thickness.

Mitral inflow velocity was measured by pulsed-wave Doppler echocardiography to indexes of LV diastolic function. The velocity waveforms were used to calculate 1) peak mitral flow velocity in early diastole (PE), peak mitral inflow velocity during LA contraction (PA), 3) ratio of PE to PA, 4) time-velocity integral of the mitral inflow velocity waveform representing early filling (Ai), 5) time-velocity integral representing LA contraction (Ai), 6) ratio of Ei/Ai, and 7) deceleration time (DCT) of early mitral inflow velocity (4).

Electrocardiographic Measurements

Lead-II of the electrocardiogram was monitored throughout the study and recorded at all specified study time points. If de-novo ventricular arrhythmias were to develop at any time during the study, the electrocardiogram would be recorded continuously. If at any time arrhythmias developed and were associated with hemodynamic compromise, drug infusion would be stopped and the study terminated for that day.

Circulating Plasma Level of Troponin-I and Nt-Pro BNP

Plasma samples were obtained at all study time points and stored at −70° C. for future use. Plasma samples from 6 normal dogs were also obtained and stored for comparison. TnI and nt-pro BNP were determined in plasma based on the principle of the double antibody sandwich enzyme-linked immunosorbent assay (ELISA). TnI and nt-pro BNP were assayed using commercially available assay kits. Kits for TnI were purchased from ALPCO Diagnostics, Salem, N.H. and kits for nt-pro BNP were purchased from Kamiya Biomedical Company (Cat# KT-23770). Using standard curves and software, the concentration of TnI was expressed as ng/ml and that of nt-pro BNP in pg/ml.

Data Analysis

The study was powered with LV ejection fraction as the primary endpoint. Within group comparisons were performed using repeated measures analysis of variance (ANOVA) with alpha set at p≤0.05. If significance was attained, pairwise comparisons between pre-treatment and 2 hours, 24 hours, 1 week and 2 weeks and between 2 hours and 24 hours, 1 week and 2 weeks were performed using the Student-Newman-Keuls Test with significance set at p<0.05. The same tests were used for analysis of TnI and nt-pro BNP. All data are reported as the mean±standard error of the mean (SEM).

Results

All 21 dogs entered into the study completed the study. None of the dogs developed de-novo ventricular or atrial arrhythmias during infusion of MST-188 or during the 2 week follow-up period. There were no clinical in-life side effects or adverse events throughout the study period.

Findings in Control Dogs

Hemodynamic, ventriculographic, and Doppler-echocardiographic results in control dogs are shown in Table 24. There were no significant changes in heart rate, systolic aortic pressure, mean aortic pressure or LV end-diastolic pressure. Both systolic and mean aortic pressures tended to increase a 1 week but the increase did not reach statistical significance. Peak LV +dP/dt and −dP/dt increased at 1 week. This increase was most likely driven by an increase in aortic blood pressure also seen at 1 week. Treatment with saline had no significant effects on CO, SV, FAS, SVR or any of the indexes of LV diastolic function namely PE/PA, Ei/Ai, DCT and LV EDWS.

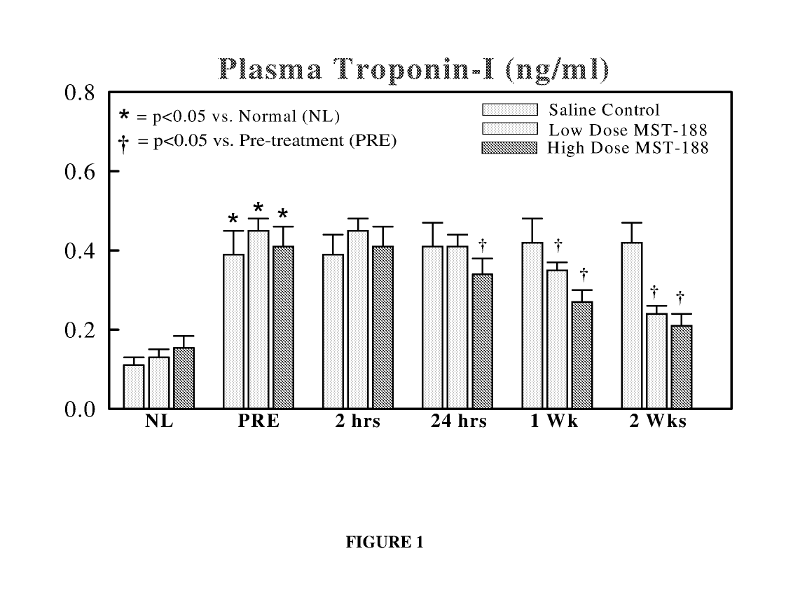

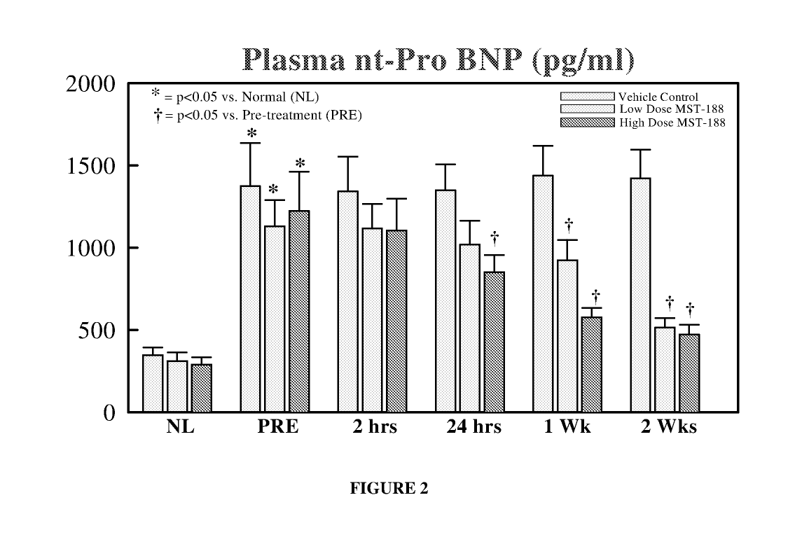

In this group, EDV tended to increase but the change did not reach statistical significance. LV ESV also tended to increase during the course of 2 weeks. The increase reached statistical significant at the 24 hours, 1 week- and 2 week-time points compared to pre-treatment (Table 24). Plasma TnI levels increased significantly at pre-treatment compared to normal levels but remained essentially unchanged thereafter compared to pre-treatment (FIG. 1). Plasma nt-pro BNP levels also increased significantly at pre-treatment compared to normal levels but remained essentially unchanged thereafter compared to pre-treatment (FIGS. 1 and 2).

TABLE 24
Hemodynamic, Ventriculographic, and Doppler-Echocardiographic Results in
Control Dogs (n = 7).

| | PRE- | | | | |
|---|---|---|---|---|---|
| | Treatment | 2 Hours | 24 Hours | 1 Week | 2 Weeks |
| Heart Rate | 82 ± 2.4 | 82 ± 1.7 | 83 ± 2.1 | 81 ± 2.0 | 82 ± 2.7 |
| (beats/min) | | | | | |
| Systolic AoP | 93 ± 2.5 | 97 ± 2.3 | 93 ± 1.7 | 100 ± 3.9 | 93 ± 2.6 |
| (mmHg) | | | | | |
| Mean AoP (mmHg) | 79 ± 2.5 | 83 ± 1.8 | 78 ± 1.3 | 84 ± 4.4 | 78 ± 3.4 |
| LV EDP (mmHg) | 14.4 ± 0.8 | 15.3 ± 1.0 | 15.1 ± 0.6† | 13.3 ± 0.5 | 13.6 ± 0.5 |
| Peak LV + dP/dt | 1264 ± 76 | 1481 ± 137 | 1353 ± 82 | 1629 ± 176* | 1396 ± 58 |
| (mmHg/sec) | | | | | |
| Peak LV − dP/dt | 1184 ± 61 | 1217 ± 69 | 1253 ± 48 | 1404 ± 97*† | 1280 ± 68 |
| (mmHg/sec) | | | | | |
| LV EDV (ml) | 70 ± 3.7 | 71 ± 3.5 | 72 ± 3.4 | 73 ± 3.4 | 72 ± 3.4 |
| LV ESV (ml) | 48 ± 2.8 | 48 ± 2.5 | 49 ± 2.5* | 50 ± 2.5* | 50 ± 2.7*† |
| LV EF (%) | 32 ± 0.6 | 32 ± 0.6 | 32 ± 0.7 | 31 ± 0.6 | 31 ± 0.8 |
| CO (L/min) | 1.86 ± 0.12 | 1.89 ± 0.12 | 1.93 ± 0.11 | 1.87 ± 0.11 | 1.80 ± 0.10 |
| SV (ml/beat) | 23 ± 1.0 | 23 ± 1.1 | 23 ± 1.0 | 23 ± 1.0 | 22 ± 1.0 |
| SVR (dynes-cm-sec⁻⁵) | 3507 ± 255 | 3629 ± 299 | 3264 ± 161 | 3693 ± 348 | 3519 ± 257 |
| LV FAS (%) | 28 ± 1.5 | 29 ± 1.5 | 29 ± 1.6 | 29 ± 1.6 | 28 ± 1.5 |
| PE/PA | 1.75 ± 0.07 | 1.67 ± 0.08 | 1.71 ± 0.09 | 1.78 ± 0.10 | 1.70 ± 0.05 |
| Ei/Ai | 4.06 ± 0.56 | 3.67 ± 0.33 | 3.80 ± 0.47 | 3.75 ± 0.35 | 3.58 ± 0.32 |
| DCT (msec) | 76 ± 2 | 77 ± 3 | 76 ± 1 | 77 ± 1 | 77 ± 2 |
| LV EDWS (g/cm²) | 66 ± 7 | 69 ± 6 | 74 ± 6 | 62 ± 5 | 66 ± 4 |
| TnI (ng/ml) | 0.39 ± 0.06 | 0.39 ± 0.05 | 0.41 ± 0.06 | 0.42 ± 0.06 | 0.42 ± 0.05 |
| nt-pro BNP (pg/ml) | 1373 ± 262 | 1342 ± 210 | 1349 ± 158 | 1438 ± 181 | 1421 ± 174 |

AoP = aortic pressure;
LV = left ventricular;
EDP = end-diastolic pressure;
EDV = end-diastolic volume;
ESV = end-systolic volume;
EF = ejection fraction;
CO = cardiac output;
SV = stroke volume;
SVR = systemic vascular resistance;
FAS = fractional area of shortening;
PE/PA = ratio of peak mitral inflow velocity in early diastole (PE) to peak mitral inflow velocity during left atrial contraction (PA);
Ei/Ai = ratio of integral of mitral inflow velocity in early diastole (Ei) to integral of mitral inflow velocity during left atrial contraction (Ai);
DCT = deceleration time of early mitral inflow velocity;
EDWS = end-diastolic circumferential wall stress;
TnI = plasma troponin-I level.
Nt-pro BNP = n-terminal pro brain natriuretic peptide.
* = p < 0.05 vs. Pre-Treatment;
† = p <0.05 vs. 2 hours.

Findings in Dogs Treated with Low Dose MST-188

Hemodynamic, ventriculographic, and Doppler-echocardiographic results in control dogs are shown in Table 25. There were no significant changes in heart rate, LV end-diastolic pressure or SVR at any of the study time points. Systolic and mean aortic pressure tended to increase and reached statistical significance at 1 week post treatment. A similar trend was seen with respect to peak LV +dP/dt and peak LV −dP/dt. Low dose MST-188 tended to increase CO and SV at all study time points compared to pre-treatment and the increase reached statistical significance at 2 hours, 24 hours and 1 week for SV and at 2 hours and 1 week for CO. FAS increased significantly at all study time points compared to pre-treatment. Indexes of LV diastolic function improved modestly for up to 1 week post-treatment. The ratio Ei/Ai increased significantly at 2 hours and 24 hours post treatment and DCT increased significantly at 2 hours post treatment.

In this group, EDV tended to decrease at all study time points but the change did not reach statistical significance. Similarly, ESV tended to decrease during the follow-up period. The decrease compared to pre-treatment was significant at 2 hours, 24 hours and 1 week. LV EF tended to increase during the follow-up period reaching significant at 2 hours, 24 hours and 1 week post-treatment (Table 25). Plasma TnI and nt-pro BNP levels which were significantly elevated at pre-treatment compared to normal levels decreased significantly at 1 week and 2 weeks after treatment compared to pre-treatment (FIGS. 1 and 2).

TABLE 25
Hemodynamic, Ventriculographic, and Doppler-Echocardiographic Results in Dogs
Treated with Low Dose MST-188 (n = 7).

| | PRE- | | | | |
|---|---|---|---|---|---|
| | Treatment | 2 Hours | 24 Hours | 1 Week | 2 Weeks |
| Heart Rate (beats/min) | 85 ± 1.5 | 85 ± 1.6 | 83 ± 2.3 | 85 ± 1.8 | 86 ± 1.4 |
| Systolic AoP (mmHg) | 89 ± 0.9 | 94 ± 2.0 | 91 ± 1.3 | 104 ± 5.9* | 100 ± 3.9 |
| Mean AoP (mmHg) | 76 ± 0.9 | 81 ± 1.7 | 77 ± 2.1 | 90 ± 6.2* | 87 ± 3.6 |
| LV EDP (mmHg) | 14 ± 0.9 | 15 ± 0.9 | 15 ± 0.7 | 15 ± 0.7 | 15 ± 0.9 |
| Peak LV + dP/dt | 1140 ± 71 | 1355 ± 67 | 1238 ± 37 | 1547 ± 113* | 1451 ± 94* |
| (mmHg/sec) | | | | | |
| Peak LV − dP/dt | 1148 ± 71 | 1187 ± 40 | 1166 ± 29 | 1344 ± 113 | 1377 ± 120 |
| (mmHg/sec) | | | | | |
| LV EDV (ml) | 70 ± 2.1 | 66 ± 1.8 | 68 ± 1.6 | 69 ± 2.0 | 68 ± 2.3 |
| LV ESV (ml) | 48 ± 2.0 | 39 ± 1.5* | 42 ± 2.1* | 42 ± 1.9* | 44 ± 1.8† |
| LV EF (%) | 31 ± 1.4 | 41 ± 1.4* | 38 ± 1.9* | 40 ± 1.9* | 35 ± 1.5† |
| CO (L/min) | 1.84 ± 0.09 | 2.27 ± 0.07* | 2.14 ± 0.11 | 2.37 ± 0.11* | 2.06 ± 0.12 |
| SV (ml/beat) | 22 ± 0.9 | 27 ± 1.1* | 26 ± 1.0* | 28 ± 1.5* | 24 ± 1.4 |
| SVR (dynes-cm-sec⁻⁵) | 3352 ± 176 | 2870 ± 93 | 2927 ± 194 | 3109 ± 287 | 3479 ± 266 |
| LV FAS (%) | 29 ± 1.6 | 35 ± 1.4* | 35 ± 1.9* | 36 ± 2.2* | 35 ± 2.9* |
| PE/PA | 1.68 ± 0.19 | 1.97 ± 0.15 | 1.88 ± 0.07 | 1.70 ± 0.13 | 1.71 ± 0.07 |
| Ei/Ai | 3.53 ± 0.32 | 4.69 ± 0.38* | 4.40 ± 0.34* | 4.21 ± 0.34 | 3.85 ± 0.34† |
| DCT (msec) | 77 ± 3 | 84 ± 2* | 83 ± 4 | 82 ± 3 | 78 ± 3 |
| LV EDWS (g/cm²) | 61 ± 7 | 65 ± 8 | 70 ± 8 | 66 ± 6 | 67 ± 7 |
| TnI (ng/ml) | 0.45 ± 0.03 | 0.45 ± 0.03 | 0.41 ± 0.03 | 0.35 ± 0.02* | 0.24 ± 0.02* |
| nt-pro BNP (pg/ml) | 1131 ± 158 | 1116 ± 150 | 1019 ± 146 | 924 ± 124* | 515 ± 58* |

Abbreviations same as in Table 24.
* = p < 0.05 vs. Pre-Treatment;
† = p < 0.05 vs. 2 hours.

Findings in Dogs Treated with High Dose MST-188

Hemodynamic, ventriculographic, and Doppler-echocardiographic results in control dogs are shown in Table 26. There were no significant changes in heart rate, LV end-diastolic pressure or SVR at any of the study time points. Systolic and mean aortic pressure tended to increase and reached statistical significance at 1 week post treatment. A similar trend was seen with respect to peak LV +dP/dt and peak LV −dP/dt. High dose MST-188 tended to increase CO and SV at all study time points compared to pre-treatment and the increase reached statistical significance at 2 hours and 1 week for SV and at 1 week for CO. FAS increased significantly at all study time points compared to pre-treatment except at 2 weeks. Indexes of LV diastolic function improved modestly for up to 1 week post-treatment. The ratio Ei/Ai increased significantly at 2 hours post treatment and DCT increased significantly at 2 hours, 24 hours and 1 week post treatment.

In this group, EDV tended to decrease at all study time points and reached significance at 24 hours post-treatment. Similarly, ESV tended to decrease during the follow-up period. The decrease compared to pre-treatment was significant at 2 hours, 24 hours and 1 week. LV EF tended to increase during the follow-up period reaching significant at 2 hours, 24 hours and 1 week post-treatment (Table 26). Plasma TnI and nt-pro BNP levels which were significantly elevated at pre-treatment compared to normal levels decreased significantly at 24 hours, 1 week and 2 weeks after treatment compared to pre-treatment (FIG. 1).

TABLE 26
Hemodynamic, Ventriculographic, and Doppler-Echocardiographic Results in Dogs
Treated with High Dose MAST-188 (n = 7).

| | PRE- | | | | |
|---|---|---|---|---|---|
| | Treatment | 2 Hours | 24 Hours | 1 Week | 2 Weeks |
| Heart Rate (beats/min) | 85 ± 1.8 | 82 ± 1.5 | 86 ± 2.0 | 88 ± 1.6 | 87 ± 1.2 |
| Systolic AoP (mmHg) | 90 ± 1.1 | 99 ± 4.5 | 95 ± 3.6 | 105 ± 4.3* | 100 ± 3.9 |
| Mean AoP (mmHg) | 78 ± 0.4 | 85 ± 4.6 | 82 ± 3.8 | 91 ± 4.3* | 88 ± 3.5 |
| LV EDP (mmHg) | 15 ± 0.5 | 14 ± 0.5 | 14 ± 0.3 | 14 ± 0.5 | 13 ± 0.5 |
| Peak LV + dP/dt | 1272 ± 104 | 1514 ± 122 | 1355 ± 117 | 1699 ± 149* | 1572 ± 121 |
| (mmHg/sec) | | | | | |
| Peak LV − dP/dt | 1162 ± 68 | 1256 ± 86 | 1191 ± 58 | 1413 ± 68* | 1354 ± 73 |
| (mmHg/sec) | | | | | |
| LV EDV (ml) | 74 ± 3.6 | 70 ± 3.4 | 70 ± 3.4* | 73 ± 3.9 | 74 ± 3.9 |
| LV ESV (ml) | 50 ± 2.6 | 41 ± 2.3* | 42 ± 2.3* | 43 ± 3.0* | 47 ± 2.8† |
| LV EF (%) | 32 ± 0.5 | 42 ± 1.6* | 40 ± 1.9* | 40 ± 2.3* | 36 ± 2.4† |
| CO (L/min) | 1.99 ± 0.9 | 2.45 ± 0.11 | 2.37 ± 0.18 | 2.56 ± 0.21* | 2.30 ± 0.23 |
| SV (ml/beat) | 23 ± 1.1 | 30 ± 1.7* | 28 ± 2.1 | 29 ± 2.4* | 27 ± 2.6 |
| SVR (dynes-cm-sec⁻⁵) | 3158 ± 140 | 2815 ± 160 | 2851 ± 206 | 2932 ± 217 | 3204 ± 295 |
| LV FAS (%) | 31 ± 1.5 | 40 ± 2.1* | 37 ± 1.5* | 35 ± 2.0* | 34 ± 2.3† |
| PE/PA | 1.67 ± 0.09 | 1.97 ± 0.10 | 1.79 ± 0.17 | 1.57 ± 0.08† | 1.72 ± 0.15 |
| Ei/Ai | 3.54 ± 0.22 | 4.56 ± 0.31* | 4.33 ± 0.35 | 3.49 ± 0.28 | 3.73 ± 0.35† |
| DCT (msec) | 73 ± 3 | 81 ± 3* | 80 ± 3* | 76 ± 3* | 75 ± 3† |
| LV EDWS (g/cm²) | 59 ± 3 | 61 ± 5 | 61 ± 5 | 60 ± 5 | 62 ± 4 |
| TnI (ng/ml) | 0.41 ± 0.05 | 0.41 ± 0.05 | 0.34 ± 0.04* | 0.27 ± 0.03* | 0.21 ± 0.03* |
| nt-pro BNP (pg/ml) | 1223 ± 239 | 1105 ± 193 | 852 ± 104* | 577 ± 58* | 472 ± 60* |

Abbreviations same as in Table 24.
* = p < 0.05 vs. Pre-Treatment;
† = p < 0.05 vs. 2 hours.

Conclusions

Results of the study indicate that in dogs with advanced systolic heart failure, intravenous administration of MST-188 over a period of 2 hours results in improved LV systolic function that is maintained for at least 1 week after cessation of drug administration. This observation is consistent with progressive reduction in plasma nt-pro BNP. LV diastolic function also tended to improve over this time course but the improvement was modest. Heart rate was essentially unchanged during each of the study time points and, therefore, the improvements in LV function could not be attributed to changes in the chronotropic state. Administration of MST-188 also had minimal or no effects on LV end-diastolic pressure, end-diastolic volume and systemic vascular resistance and, therefore, the improvements in LV function could not be attributed to vasodilation namely alteration in cardiac loading conditions. Furthermore, systemic blood pressure did not fall but rather increased suggesting increased LV stroke output in the absence of a change in vascular resistance.

Experiments conducted as part of the present study did not address possible mechanisms of action of MST-188. Calcium overload occurs in heart failure leading to cardiomyocyte dysfunction and death. MST-188 (purified poloxamer 188) is a cardioprotective and rheologic agent that was shown to improve LV function and reduce reinfarction in myocardial infarction. Its activity results from repair of damaged cell membranes, possibly inhibiting unregulated calcium entry into cardiomyocytes and/or improved microvascular blood flow. It is also possible that MST-188 prevented/minimized calcium overload in cardiomyocyte thus preventing secondary LV dysfunction via ongoing death and dysfunction of cardiomyoctes. This is supported, in part, by reduced circulating levels of TnI, a biomarker of cardiomyocyte injury and death. It also is possible that MST-188 improved myocardial perfusion and oxygenation (relief of regional ischemia/hypoxia) via improved microvascular blood flow leading to improved LV function.

### Example 19

Stability of Salt Free Formulation

LCMF poloxamer 188 Injection, 22.5% clinical drug product of Example 1 has been studied for 6 months at the recommended storage condition of 5° C.±3° C. ambient RH and the alternative storage condition of 25° C.±2° C./60%±5% RH. The results indicate that the product is stable at both conditions. It is expected that the product will remain usable for 24 months when stored at the recommended storage condition of 5° C.±3° C. ambient RH.

TABLE 27
Stability Data for Vepoloxamer Drug Product Lot MTH15006 Stored
at Long-term Condition
5° C. ± 3° C./Ambient Relative Humidity

| | Acceptance | | 1 | 3 | 6 | 9 | 12 | 18 | 24 |
|---|---|---|---|---|---|---|---|---|---|
| Test Attribute | Criteria | Initial | months | months | months | months | months | months | months |
| Appearance | Clear, colorless, | Conform | Conform | Conform | Conform | | | | |
| | homogenous | | | | | | | | |
| | solution | | | | | | | | |
| | essentially free | | | | | | | | |
| | from visible | | | | | | | | |
| | particulate | | | | | | | | |
| | matter which | | | | | | | | |
| | foams on | | | | | | | | |
| | shaking | | | | | | | | |
| Container Closure | Conforms | Conform | NS | NS | NS | | | | |
| Integrity | | | | | | | | | |
| Assay (% LC) | 90-110% of | 106 | 100 | 99 | 100 | | | | |
| | label claim | | | | | | | | |
| | (equivalent to | | | | | | | | |
| | 135 to 165 | | | | | | | | |
| | mg/mL) | | | | | | | | |
| Molecular Weight | | | | | | | | | |
| Peak MW (Da) | 8500 ± 1000 Da | 8252 | 8351 | 8418 | 8491 | | | | |
| Weight Average | 8500 ± 1000 Da | 8069 | 8190 | 8284 | 8475 | | | | |
| MW (Da) | | | | | | | | | |
| % LMW | NMT 2.0% | 0.91 | 1.3 | 0.83 | 1.14 | | | | |
| (<4,5000 Da) | | | | | | | | | |
| % HMW | NMT 2.0% | 0.79 | 0.91 | 0.88 | 1.00 | | | | |
| (>13,000 Da) | | | | | | | | | |
| Polydispersity | NMT 1.05 | 1.03 | 1.03 | 1.03 | 1.03 | | | | |

Glycols (ppm)

| | | | | | |
|---|---|---|---|---|---|
| Ethylene glycol | NMT 30 ppm | <10 | <25 | <25 | <25 |
| Diethylene glycol | NMT 30 ppm | <10 | <25 | <25 | <25 |
| Triethylene glycol | NMT 30 ppm | <10 | <25 | <25 | <25 |
| Propylene glycol | NMT 30 ppm | <10 | <25 | <25 | <25 |

NLT = not less than; NMT = not more than; MW = molecular weight; Da = Daltons; LMW = low molecular
weight; HMW = high molecular weight; ppm = parts per million, NS = Not scheduled
Volatile Degradation Products (ppm)

| | | | | | |
|---|---|---|---|---|---|
| Methanol | NMT 300 ppm | 69. | 70 | 81 | 74 |
| Acetone | NMT 300 ppm | 2. | 1 | 1 | 1 |
| Acetaldehyde | NMT 300 ppm | 84 | 103 | 46 | 102 |
| Propionaldehyde | NMT 300 ppm | 15 | 21 | 34 | 46 |
| Formaldehyde | NMT 100 ppm | <30 | <30 | <30 | <30 |
| (ppm) | | | | | |
| pH | 5.0-6.4 | 6.0 | 6.0 | 5.9 | 5.9 |
| Osmolality | Report result | 405 | NS | NS | NS |
| | mmol/kg | | | | |
| Sub Visible | ≥10 μm NMT | 7 | NS | NS | NS |
| Particles | 6000/container | | | | |
| | ≥25 μm NMT | 0 | NS | NS | NS |
| | 6000/container | | | | |
| Sterility | Sterile | Conform | NS | NS | NS |
| Bacterial | NMT 3.30 | <0.05 | NS | NS | NS |
| Endotoxins | EU/mL | | | | |
| (EU/mg) | | | | | |

ppm = parts per million; NMT = not more than; EU = endotoxin unit; NS = Not scheduled

TABLE 29
Stability Data for Vepoloxamer Drug Product Lot MTH15006 Stored
at Accelerated Condition
25° C. ± 2° C./60% ± 5% Relative Humidity

| | Acceptance | | 1 | 3 | 6 | 9 | 12 | 18 | 24 |
|---|---|---|---|---|---|---|---|---|---|
| Test Attribute | Criteria | Initial | months | months | months | months | months | months | months |
| Appearance | Clear, colorless, | Conform | Conform | Conform | Conform | | | | |
| | homogenous | | | | | | | | |
| | solution | | | | | | | | |
| | essentially free | | | | | | | | |
| | from visible | | | | | | | | |
| | particulate | | | | | | | | |
| | matter which | | | | | | | | |
| | foams on | | | | | | | | |
| | shaking | | | | | | | | |
| Container Closure | Conforms | Conform | NS | NS | NS | | | | |
| Integrity | | | | | | | | | |
| Assay (% LC) | 90-110% of | 106 | 99 | 100 | 99 | | | | |
| | label claim | | | | | | | | |
| | (equivalent to | | | | | | | | |
| | 135 to 165 | | | | | | | | |
| | mg/mL) | | | | | | | | |
| Molecular Weight | | | | | | | | | |
| Peak MW (Da) | 8500 ± 1000 Da | 8252 | 8364 | 8439 | 8493 | | | | |
| Weight Average | 8500 ± 1000 Da | 8069 | 8196 | 8273 | 8463 | | | | |
| MW (Da) | | | | | | | | | |
| % LMW | NMT 2.0% | 0.91 | 1.1 | 1.1 | 1.11 | | | | |
| (<4,5000 Da) | | | | | | | | | |
| % HMW | NMT 2.0% | 0.79 | 0.84 | 0.86 | 0.92 | | | | |
| (>13,000 Da) | | | | | | | | | |
| Polydispersity | NMT 1.05 | 1.03 | 1.03 | 1.03 | 1.03 | | | | |

Glycols (ppm)

| | | | | | |
|---|---|---|---|---|---|
| Ethylene glycol | NMT 30 ppm | <10 | <25 | <25 | <25 |
| Diethylene glycol | NMT 30 ppm | <10 | <25 | <25 | <25 |
| Triethylene glycol | NMT 30 ppm | <10 | <25 | <25 | <25 |
| Propylene glycol | NMT 30 ppm | <10 | <25 | <25 | <25 |

NLT = not less than; NMT = not more than; MW = molecular weight; Da = Daltons; LMW = low molecular
weight; HMW = high molecular weight; ppm = parts per million, NS = Not scheduled
Volatile Degradation Products (ppm)

| | | | | | |
|---|---|---|---|---|---|
| Methanol | NMT 300 ppm | 69. | 70 | 80 | 73 |
| Acetone | NMT 300 ppm | 2. | 2 | 1 | 2 |
| Acetaldehyde | NMT 300 ppm | 84 | 152 | 49 | 99 |
| Propionaldehyde | NMT 300 ppm | 15 | 48 | 59 | 63 |
| Formaldehyde | NMT 100 ppm | <30 | <30 | <30 | <30 |
| (ppm) | | | | | |
| pH | 5.0-6.4 | 6.0 | 6.0 | 5.9 | 5.9 |
| Osmolality | Report result | 405 | NS | NS | NS |
| | mmol/kg | | | | |
| Sub Visible | ≥10 μm NMT | 7 | NS | NS | NS |
| Particles | 6000/container | | | | |
| | >25 μm NMT | 0 | NS | NS | NS |
| | 600/container | | | | |
| Sterility | Sterile | Conform | NS | NS | NS |
| Bacterial | NMT 3.30 | <0.05 | NS | NS | NS |
| Endotoxins | EU/mL | | | | |
| (EU/mg) | | | | | |

ppm = parts per million; NMT = not more than; EU = endotoxin unit; NS = Not scheduled

### Example 20

Methods

Detection of Sodium:

The presence of sodium (Na) in poloxamer 188 Injection, 22.5%, was analyzed as a possible impurity by inductively coupled plasma emission spectrometry (ICP-OES). The formulation sample was prepared as well as a set of spikes with known amounts of Na intentionally added were examined against a calibration curve and results were calculated.

Reagents used:
High Purity Water (18 megohm or greater resistivity), Millipore Milli-Q Gradient A10 Water System;
Trace Elements Grade Concentrated Hydrochloric Acid (HCl), Fisher Scientific; and
Single Element Standard, Custom Grade, NIST traceable, from Inorganic Ventures, Inc.:
   Sodium=998 μg/mL

Equipment:
Millipore Milli-Q Gradient A10 Water System
Mettler AG204 Balance
Mettler XS205DU
Thermo Scientific 6300 Duo ICP Spectrometer

Procedure:

Intermediate Standard:

Diluted 1.0 HCl and 0.5 mL Na reference standard to 50 mL in a centrifuge tube with H₂O. Also prepared a check intermediate standard in identical manner. Na=9.98 μg/mL

Calibration Standards:

Prepared according to the following Table 31 in centrifuge tubes. Diluted to 100 mL with H₂O and mixed well.

TABLE 31
preparation of calibration standards

| | mL | mL | Final mL | Na, |
|---|---|---|---|---|
| ID | HCl | Intermediate | H₂O | μg/mL |
| Cal Blk | 1 | 0 | 50 | 0 |
| Standard 1 | 2 | 0.25 | 100 | 0.0250 |
| Standard 2 | 1 | 0.25 | 50 | 0.0499 |
| Standard 3 | 1 | 0.5 | 50 | 0.0998 |
| Standard 4 | 1 | 2.5 | 50 | 0.499 |
| Standard 5 | 1 | 5.0 | 50 | 0.998 |

Sample Preparation

In triplicate, 5.0 mL sample and 1.0 mL HCl were transferred to a 50-mL centrifuge tube, diluted to volume with H₂O, and mixed well. A set of spikes were also prepared, with known amounts of intermediate standard added prior to dilution to volume with H₂O. See Table 32 below for a summary of the sample and spike preparations.

TABLE 32
Sample and Spike Preparation

| | | | mL | Final | Na |
|---|---|---|---|---|---|
| | mL | mL | Intermediate | mL | Spike, |
| ID | Sample | HCl | Standard | H₂O | μg/mL |
| Dig Blk | 0 | 1.0 | 0 | 50 | 0 |
| SSADV-01 A | 5.0 | 1.0 | 0 | 50 | 0 |
| SSADV-01 B | 5.0 | 1.0 | 0 | 50 | 0 |
| SSADV-01C | 5.0 | 1.0 | 0 | 50 | 0 |
| Spike A1 | 5.0 | 1.0 | 0.576 | 50 | 0.115 |
| Spike A2 | 5.0 | 1.0 | 0.576 | 50 | 0.115 |
| Spike A3 | 5.0 | 1.0 | 0.576 | 50 | 0.115 |
| Spike B1 | 5.0 | 1.0 | 1.15 | 50 | 0.230 |
| Spike B2 | 5.0 | 1.0 | 1.15 | 50 | 0.230 |
| Spike B3 | 5.0 | 1.0 | 1.15 | 50 | 0.230 |
| Spike C1 | 5.0 | 1.0 | 1.73 | 50 | 0.345 |
| Spike C2 | 5.0 | 1.0 | 1.73 | 50 | 0.345 |
| Spike C3 | 5.0 | 1.0 | 1.73 | 50 | 0.345 |

Instrumental Analysis:

Results were generated using the instrumental conditions below:

Element/Wavelength Settings:

| Element | Wavelength (nm) |
|---|---|
| Na | 589.592 |

Plasma and Torch Parameters:
Auxiliary Gas Flow (L/min): 0.5
Plasma View: Radial
RF Power (kW): 1.15
Nebulizer Flow (L/min): 0.70
Flush Pump Rate (rpm): 100
Analysis Pump Rate (rpm): 100
Number Repeats: 3
Delay Time (s): 0
Sample Flush Time (s): 45
Low Wavelength Range (s): 10
High Wavelength Range (s): 1
Nebulizer: Conikal
Spray Chamber: Cyclonic
Sample Tubing: Tygon-Orange/White
Drain Tubing: Tygon-White/White

Analysis Procedure

Calibration and Quality Control:

The integrated area of the emission peak is used as the analytical signal. Suitable background correction positions are 1 on the left and 12 on the right. These positions may be changed or set automatically by the instrument software if conditions require.

System Suitability: The correlation coefficient of the calibration curve was ≥0.995 and standards interspersed within the run recovered 100±5%.

Calculations:

Calculations:

The content of Na in unspiked samples was calculated as follows:

$$Na ppm = Na , μ g \text{/} mL \times \frac{DilVol mL}{Aliquot mL}$$

Spike recovery was calculated, incorporating the sodium result generated in the unspiked sample, in the total:

$$Na \% Recovery = ( \frac{Spiked Sample Na , ppm - Sample Na , ppm}{Na Added , ppm} ) \times 100$$

ppm Na was converted to mmol Na/dose as follows:

$$Na mmol \text{/} dose = Na , μ g \text{/} mL \times \frac{1 μ mol Na}{22.9898 μ g Na} \times \frac{1 mmol Na}{1000 μ mol Na} \times \frac{100 mL}{Dose}$$

### REFERENCES

1. Sabbah H N, Stein P D, Kono T, Gheorghiade M, Levine T B, Jafri S, et al. A canine model of chronic heart failure produced by multiple sequential coronary microembolizations. Am J Physiol. 1991; 260:H1379-84.
2. Dodge H T, Sandler H, Baxley W A, Hawley R R. Usefulness and limitations of radiographic methods for determining left ventricular volume. The American journal of cardiology. 1966; 18:10-24.
3. Sabbah H N, Imai M, Cowart D, Amato A, Carminati P, Gheorghiade M. Hemodynamic properties of a new-generation positive luso-inotropic agent for the acute treatment of advanced heart failure. The American journal of cardiology. 2007; 99:41A-46A.
4. Rastogi S, Guerrero M, Wang M, Ilsar I, Sabbah M S, Gupta R C, Sabbah H N. Myocardial transfection with naked DNA plasmid encoding hepatocyte growth factor prevents the progression of heart failure in dogs. American journal of physiology Heart and circulatory physiology. 2011; 300:H1501-H1509.

Various modifications, additions, substitutions, and variations to the illustrative examples set forth herein will be apparent to those skilled in the art from the foregoing description. Such modifications are also intended to fall within the scope of the appended claims.

The various features and embodiments of the present invention, referred to in individual sections above apply, as appropriate, to other sections, mutatis mutandis. Consequently features specified in one section may be combined with features specified in other sections, as appropriate.

1. A sterile, injectable solution comprising: poloxamer 188 and water for injection, wherein
the sterile, injectable solution is reduced in sodium or substantially sodium-free;
the poloxamer 188 is at a concentration greater than 15% w/v; and
the sterile, injectable solution has a pH of from about 4 to about 8.

2. The solution of claim 1, wherein the solution does not cause clinically significant complement activation when administered to a patient as evidenced by clinical symptoms and signs selected from one or more from the group consisting of hypotension, tachycardia, and shortness of breath.

3. The solution of claim 1, wherein the poloxamer 188 is poloxamer 188, N.F.

4. The solution of claim 1, wherein the poloxamer 188 is purified poloxamer 188.

5. The solution of claim 1, wherein the poloxamer 188 is a long circulating material free (LCMF) poloxamer 188 having the formula
HO(CH₂CH₂O)_{a′}—[CH(CH₃)CH₂O]_{b}—(CH₂CH₂O)ₐH;
each of a and a′ is an integer such that the percentage of the hydrophile (C₂H₄O) is between approximately 60% and 90% by weight of the total molecular weight of the copolymer;
a and a′ are the same or different;
b is an integer such that the molecular weight of the hydrophobe [CH(CH₃)CH₂O]_{b} is between approximately 1,300 to 2,300 Daltons;
no more than 1.5% of the total components in the distribution of the co-polymer are low molecular weight components having an average molecular weight of less than 4,500 Daltons;
no more than 1.5% of the total components in the distribution of the co-polymer are high molecular weight components having an average molecular weight of greater than 13,000 Daltons;
the polydispersity value of the copolymer is less than approximately 1.07 or less than 1.07; and
the circulating half-life of the co-polymer, when administered to a subject, is no more than 5.0-fold longer than the circulating half-life of the main component in the distribution of the co-polymer.

6. The solution of claim 5, wherein the poloxamer 188 is produced by a method comprising:
admixing a solution of poloxamer 188 in a first alkanol with an extraction solvent comprising a second alkanol and supercritical carbon dioxide under a temperature and pressure to maintain the supercritical carbon dioxide for a first defined period, wherein:
the temperature is above the critical temperature of carbon dioxide but is no more than 40° C.;
the pressure is 220 bars to 280 bars; and
the alkanol is provided at an alkanol concentration that is 7% to 8% by weight of the total extraction solvent; and
increasing the concentration of the second alkanol in the extraction solvent a plurality of times in gradient steps over time of the extraction method, wherein:
each plurality of times occurs for a further defined period; and
in each successive step, the alkanol concentration is increased 1-2% compared to the previous concentration of the second alkanol; and
removing the extraction solvent from the extractor vessel to thereby remove the extracted material from the poloxamer preparation.

7. The solution of claim 1, wherein the poloxamer 188 is present at a concentration of greater than about 15% w/v up to about 30% w/v.

8.-13. (canceled)

14. The solution of claim 1, wherein the solution further comprises one or more tonicity agents.

15. The solution of claim 14, wherein said one or more tonicity agents are selected from the group consisting of glucose, glycerin (glycerol), dextrose, sucrose, xylitol, fructose, mannitol, sorbitol, mannose, potassium salts, calcium salts, and magnesium salts.

16. The solution of claim 15, wherein the tonicity agent is a magnesium salt.

17. The solution of claim 16, wherein the magnesium salt is selected from the group consisting of magnesium acetate, magnesium aluminate, magnesium borate, magnesium bicarbonate, magnesium carbonate, magnesium chloride, magnesium citrate, magnesium gluconate, magnesium hydroxide, magnesium lactate, magnesium metasilicate aluminate, magnesium oxide, magnesium phthalate, magnesium phosphate, magnesium silicate, magnesium stearate, magnesium succinate, magnesium tartrate, and mixtures thereof.

18. The solution of claim 17, wherein the magnesium salt is magnesium chloride.

19. The solution of claim 18, wherein the magnesium chloride is magnesium chloride hexahydrate.

20. The solution of claim 14, wherein the concentration of said one or more tonicity agents is from about 1 mM to about 20 mM.

21.-23. (canceled)

24. The solution of claim 20, wherein the solution further comprises an antioxidant.

25. The solution of claim 24, wherein the antioxidant is selected from the group consisting of one or more of cysteine, citric acid, dextrose, dithiothreitol, histidine, malic acid, mannitol, methionine, metabisulfate, and tartaric acid.

26. The solution of claim 25, wherein the antioxidant is citric acid.

27. The solution of claim 24, wherein the concentration of the antioxidant is from about 0.001% to about 2%.

28. The solution of claim 27, wherein the concentration of the antioxidant is from about 0.1 mM to about 10 mM.

29. The solution of claim 28, wherein the solution has a pH of from about 6 to about 8.

30. The solution of claim 29, wherein the solution further comprises a buffer.

31. The solution of claim 30, wherein the buffer is selected from the group consisting of citrate buffer (pH about 2); citrate buffer (pH about 5); citrate buffer (pH about 6.3); phosphate buffer (pH about 7.2); phosphate buffer (pH about 9); borate buffer (pH about 9); borate buffer (pH about 10); succinate buffer (pH about 5.6); histidine buffer (pH about 6.1); carbonate buffer (pH about 6.3); acetate buffer (pH about 7.2), meglumine, and combinations thereof.

32. The solution of claim 31, wherein the buffer comprises citric acid and meglumine at an adjusted pH of about 6.

33. The solution of claim 31, wherein the solution further comprises a pH adjusting agent.

34. The solution of claim 33, wherein the pH adjusting agent is selected from the group consisting of aqueous HCl, ammonium hydroxide, meglumine and mixtures thereof.

35. The solution of claim 1, comprising poloxamer 188 at a concentration of about 225 mg/mL and further comprising magnesium chloride hexahydrate at a concentration of about 0.610 mg/mL.

36. The solution of claim 1, wherein the osmolality of the solution is between about 100 and about 2000 mOSm/kg.

37.-49. (canceled)

50. A method of treating a disease or condition in a subject, comprising administering a therapeutically effective amount of the solution of claim 1, wherein the disease or condition is selected from acute coronary syndromes, limb ischemia, shock, stroke, heart failure, coronary artery disease, muscular dystrophy, circulatory diseases, pathologic hydrophobic interactions in blood, inflammation, sickle cell disease, venous occlusive crisis, acute chest syndrome, inflammation, pain, neurodegenerative diseases, macular degeneration, thrombosis, kidney failure, burns, spinal cord injuries, ischemic/reperfusion injury, myocardial infarction, hemo-concentration, amyloid oligomer toxicity, diabetic retinopathy, diabetic peripheral vascular disease, sudden hearing loss, peripheral vascular disease, cerebral ischemia, transient ischemic attacks, critical limb ischemia, respiratory distress syndrome (RDS), and adult respiratory distress syndrome (ARDS).

51.-57. (canceled)

58. The solution of claim 34, wherein one or more of the tonicity agent, antioxidant, buffer, or pH adjusting agent are substantially free of sodium.

59. A pharmaceutical composition for injection comprising
a) poloxamer 188
b) water;
and
c) one or more buffers selected from the group consisting of citrate buffer (pH about 2); citrate buffer (pH about 5); citrate buffer (pH about 6.3); phosphate buffer (pH about 7.2); phosphate buffer (pH about 9); borate buffer (pH about 9); borate buffer (pH about 10); succinate buffer (pH about 5.6); histidine buffer (pH about 6.1); carbonate buffer (pH about 6.3); acetate buffer (pH about 7.2), and meglumine;
wherein the amount of sodium is less than or equal to about 1.5 mg/ml.

60.-68. (canceled)

69. A method of treating a disease or condition in a subject, comprising administering the pharmaceutical composition of claim 36 to a subject in need of treatment, wherein the disease or condition is selected from acute coronary syndromes, limb ischemia, shock, stroke, heart failure, coronary artery disease, muscular dystrophy, circulatory diseases, pathologic hydrophobic interactions in blood, inflammation, sickle cell disease, venous occlusive crisis, acute chest syndrome, inflammation, pain, neurodegenerative diseases, macular degeneration, thrombosis, kidney failure, burns, spinal cord injuries, ischemic/reperfusion injury, myocardial infarction, hemo-concentration, amyloid oligomer toxicity, diabetic retinopathy, diabetic peripheral vascular disease, sudden hearing loss, peripheral vascular disease, cerebral ischemia, transient ischemic attacks, critical limb ischemia, respiratory distress syndrome (RDS), and adult respiratory distress syndrome (ARDS).

70.-83. (canceled)

84. The solution of claim 1, wherein the stability of the solution at 5° C.±3° C. is at least six months.
